SEQUENCE LISTING (10 sequences)

SEQ ID NO: 1 - DNA, 32 bp, synthetic construct
  source 1..32: mol_type = other DNA
  misc_feature 1..32: note = Fwd_1
cacttgagattcttgacattacaccatgttct

SEQ ID NO: 2 - DNA, 30 bp, synthetic construct
  source 1..30: mol_type = other DNA
  misc_feature 1..30: note = Fwd_2
cagtgtatctggaaagcctacaggacacca

SEQ ID NO: 3 - DNA, 29 bp, synthetic construct
  source 1..29: mol_type = other DNA
  misc_feature 1..29: note = Rev_1
aacaaatcttattttcgaatacgtacacg

SEQ ID NO: 4 - DNA, 30 bp, synthetic construct
  source 1..30: mol_type = other DNA
  misc_feature 1..30: note = Rev_2
ttgatacattcggtctcgtgtatcttctag

SEQ ID NO: 5 - DNA, 25 bp, synthetic construct
  source 1..25: mol_type = other DNA
  misc_feature 1..25: note = Probe_1
agtactgatatgtccctgattggtg

SEQ ID NO: 6 - DNA, 60 bp, synthetic construct
  source 1..60: mol_type = other DNA
  misc_feature 1..60: note = Probe_2
acaccatgttctgtagtactgatatgtccctgattggtgaacgtgtacgtattcgaaaat

SEQ ID NO: 7 - DNA, 100 bp, synthetic construct
  source 1..100: mol_type = other DNA
  misc_feature 1..100: note = Hom_1
acagacacttgagattcttgacattacaccatgttctgtagtactgatatgtccctgattggtgaacgtgtacgtattcgaaaataagatttgtttctcg

SEQ ID NO: 8 - DNA, 140 bp, synthetic construct
  source 1..140: mol_type = other DNA
  misc_feature 1..140: note = Non_1
cagtgtatctggaaagcctacaggacaccaaaataaccttaatcatcaattggttacaggaggctttaagttcagcatctttggctcacatgaaggccaaattccgagagaccctagaagatacacgagaccgaatgtat

SEQ ID NO: 9 - DNA, 32 bp, synthetic construct; identical to SEQ ID NO: 1
  source 1..32: mol_type = other DNA
  misc_feature 1: note = 5'FAM
  misc_feature 1..32: note = 5'FAM-Fwd_1

SEQ ID NO: 10 - DNA, 32 bp, synthetic construct
  source 1..32: mol_type = other DNA
  misc_feature 32: note = 3'BHQ1
  misc_feature 1..32: note = AntiFwd_1-3'BHQ1
agaacatggtgtaatgtcaagaatctcaagtg

(19) United States
(12) Patent Application Publication
(10) Pub. No.: US 2026/0357329 A1
(43) Pub. Date: Nov. 12, 2026

(54) METHODS AND DEVICE FOR MULTIPLE-LABEL NUCLEIC ACID AMPLIFICATION AND DETECTION

(71) Applicant: RAPIDEMIC B.V, Leiden (NL)

(72) Inventors: Violette DEFOURT, Leiden (NL); Amangeldy URAZOV, Leiden (NL); Diego PESCE, Leiden (NL)

(21) Appl. No.: 19/114,153

(22) PCT Filed: Sep. 25, 2023
(86) PCT No.: PCT/NL2023/050499

§ 371 Date: Mar. 21, 2025

(30) Foreign Application Priority Data
Sep. 23, 2022 (NL) 2033124

(51) Int. Cl. C12Q 1/6844; C12Q 1/6839

(52) U.S. Cl. CPC C12Q 1/6844; C12Q 1/6839

(57) ABSTRACT

This disclosure describes methods, compositions, kits, and uses for nucleic acid amplification and subsequent detection of the nucleic acid amplification product, using labeled nucleotides and a differently labeled probe or primer. In the disclosure, the properties of a recombinase and a separate labeled probe are used. The disclosed methods, compositions, and kits enable more sensitive and specific detection of nucleic acid amplification products at low temperatures, thereby offering improved means for nucleic acid detection in low-resource settings.

### FIELD OF THE INVENTION

The present invention relates to methods and systems for nucleic acid amplification, preferably isothermal nucleic acid amplification such as recombinase polymerase amplification, using labeled nucleotides and a differently labeled probe or primer. In addition, the invention relates to methods and systems for detection of the amplified nucleic acids using a recombinase, a labeled probe, and optionally a means for modulating binding of the recombinase or the labeled probe to the amplified nucleic acids. The invention also relates to the use of the methods and systems in healthcare, industry, and research.

### BACKGROUND OF THE INVENTION

The development of methods of isothermal nucleic acid amplification as an alternative to polymerase chain reaction (PCR) is rapidly progressing. Unlike PCR, isothermal nucleic acid amplification has the advantage that it can be carried out at a constant, and optionally ambient, temperature and does not require cyclic temperature changes. Further, isothermal nucleic acid amplification and subsequent detection methods require less resources and are simpler to perform, in many cases not requiring the use of laboratory or specialized personnel.

One such isothermal amplification method is recombinase polymerase amplification (RPA) (WO9117267A1 and Piepenburg et al., PLoS Biol. 2006 July; 4(7):e204). The RPA method uses a recombinase and a DNA polymerase together with one or more oligonucleotide primers and optional facilitating DNA-binding proteins to achieve DNA amplification. A recombinase-primer complex is formed in tandem with co-factors, which unwinds the two strands of the DNA duplex at a DNA sequence complementary to the primer. A strand-displacement polymerase subsequently elongates the primer, thereby amplifying the target sequence. One of the advantages of RPA over PCR is that the efficacy of RPA is less affected by compounds that can be found in biological samples and normally inhibit PCR.

Like PCR and isothermal amplification methods, RPA has been used for the amplification and detection of DNA and RNA of various organisms. Detection of nucleic acids is important in many industries such as the healthcare, food, and pharmaceutical industries since determination of the absence or presence of these nucleic acids provides valuable information. For example, the absence of specific nucleic acids may be necessary for the production of certain drugs or foodstuffs, while the presence of nucleic acids of pathogens assists in the diagnosis of disease or determination of the spread of these pathogens. However, a disadvantage of RPA is the generation of non-specific amplification products, which depends on the primers used for amplification. General guidelines for the primer design are known. However, they are insufficient and no primer design software can accurately predict the primer pair which will perform best. Thus, there is a need for improved methods to detect nucleic acids.

Amplification by PCR and isothermal amplification methods, for example RPA, can be monitored by various detection methods, either via end point detection following amplification or via real time detection during amplification and may be achieved by using probes, e.g. oligonucleotide and/or fluorescent probes (Lobato and O'Sullivan, Trends Analyt Chem. 2018 January; 98: p 19-35). Oligonucleotide probes comprise a reference sequence and therefore these probes can be used to verify accurate specificity of an amplification product in order to reduce the chance of non-specific detection.

End point detection is often performed via lateral flow devices or strips, while real time detection is commonly performed via fluorescent probes and fluorometers. Popular detection methods for RPA are provided by the TwistAmp® kits of TwistDx, which involve the use of a labeled oligonucleotide probe and a nuclease targeting the probe (WO2010141940A1 and Piepenburg et al.). TwistAmp® technology uses a probe-based detection of DNA during recombinase polymerase amplification. The technology utilizes a heavily modified single-stranded DNA probe which is cleaved by a restriction enzyme and becomes fluorescent (Jia et al., Archives of Virology, 2020). Such a probe can also be elongated essentially becoming a second primer which yields an amplified piece of dsDNA visualized on a lateral flow strip (Zou et al., Journal of Virological Methods, 2022).

However, these methods have several disadvantages. For instance, lateral flow strips often use gold nanoparticles and antibodies, which increase the cost of the assay. TwistAmp® kits introduce a specific probe design which includes multiple unnatural modifications and the use of a specific nuclease. These aspects of the TwistAmp® technology hinder the optimization of diagnostics assays due to complexities in designing and screening for the best probe. Real time detection requires advanced equipment for read-out. These disadvantages are exacerbated in low-resource settings. Hence, particularly for low-resource settings, there remains a need for simple, cost-efficient, and/or improved detection methods for use with nucleic acid amplification reactions, in particular isothermal amplification reactions such as RPA. Due to the complexity of RPA, it has been difficult to improve. However, because RPA is performed at low temperatures, there remains a need for increased specificity of the method.

### SUMMARY OF THE INVENTION

The inventors have surprisingly found that an amplified DNA product having a target DNA sequence (an amplicon) can be specifically detected via simpler methods compared to the current state of the art. The invention comprises using sequence-specific labeled probes without the need for exotic enzymes and expensive probe modifications. The labeled probes are incubated with recombinase and a nucleic acid amplification product, followed by detection of the resulting triple-stranded DNA/recombinase complex via for example an antibody targeting the label and attached to a solid support, such as a lateral flow strip. The surprising finding follows from the prior art indicating that it is impossible to detect the complex as such. EP0612352A1 discloses that a stable probe-dsDNA complex could only form upon formation of a double D loop (comprising a double-stranded probe, thereby forming quadruple-stranded DNA) and the subsequent removal of recombinase from the complex. U.S. Pat. No. 6,132,972A and US2003/044819A1 disclose that a probe-dsDNA complex should be cleaved with nucleases (similar to the TwistAmp® probe design) and deproteinized for detection, e.g. by using proteases. Hence, the present invention dispenses with the need of enzymatic post-processing for detection of triple-stranded DNA complexes, while only using a single-stranded labelled probe and a recombinase. Screening for the best probe can preferably be performed without chemical modifications to the probe backbone and can for example be performed on a simple agarose gel wherein the intensity of the smear would correlate with the strength of probe binding to the target dsDNA. This significantly reduces the time and cost associated with probe screening.

In addition, the inventors have surprisingly found that an amplified DNA product having a target DNA sequence (an amplicon) can be detected with higher specificity and sensitivity compared to current methods in the art. This is achieved through the incorporation of preferably a plurality of deoxynucleoside triphosphate (dNTP) monomers comprising a first label, e.g. biotin, in the amplicon, binding to the amplicon of an oligonucleotide probe comprising a nucleic acid sequence complementary to the target DNA sequence, and incorporation of a second label different from the first label, e.g. another hapten. The probe binds to the target DNA sequence with the aid of a recombinase. Either the first label or the second label is located on the probe. The second label is present on one of the primers used for amplification if the first label is present (preferably in multiple) on the probe. The probe may then also comprise (part of) the sequence of one of the other primers used for amplification. When the second label is present on the probe, the first label is incorporated (preferably in multiple) in the amplicon. The probe may then also comprise (part of) the sequence of one of the primers used for amplification. The probe and/or recombinase may be added after a nucleic acid amplification reaction or they may be added before or during, thereby enabling a so-called one pot reaction. See FIGS. 1 and 2 for a schematic illustration of the concepts as set out above. The oligonucleotide probe according to the invention does not need to be complementary to the full target DNA sequence, but may for example be complementary to a part of the target DNA sequence.

A plurality of first labels enables easier detection of the amplicon through various means, e.g. via a lateral flow strip, hence increasing sensitivity of detection of a target DNA sequence, thereby reducing components needed for detection. The use of two different labels and a probe increases the specificity of detection of the target DNA sequence, since the probe, which comprises a different label, targets the target DNA sequence in the amplified nucleic acid product independent from the primers that were used to amplify the target DNA sequence. The recombinase enables binding of the probe to be performed at low temperatures, e.g. from 20 to 50 °C, without the use of advanced equipment.

Hence, the disclosed methods, compositions, and kits enable more sensitive and specific detection of nucleic acid amplification products at low temperatures, thereby offering improved means for nucleic acid detection in low-resource settings.

The object of the present invention is therefore to provide a method of determining the presence or absence of a target deoxyribonucleic acid (DNA) sequence in a sample, the method comprising:
a. contacting the sample with:
   a first primer and a second primer for amplifying the target DNA sequence;
   a recombinase;
   a DNA-dependent DNA polymerase;
   deoxynucleoside triphosphate (dNTP) monomers comprising a first label; and
   an oligonucleotide probe comprising a nucleic acid sequence complementary to the target DNA sequence and a second label;
b. amplifying the target DNA sequence to produce a nucleic acid amplification product; and
c. detecting the nucleic acid amplification product.

It is a further object to provide a method of determining the presence or absence of a target DNA sequence in a sample, the method comprising:
a. contacting the sample with:
   a first primer and a second primer for amplifying the target DNA sequence, wherein the second primer comprises a second label and hybridizes to a first strand of DNA of the target DNA sequence;
   a recombinase;
   a DNA-dependent DNA polymerase; and
   an oligonucleotide probe comprising a nucleic acid sequence complementary to the target DNA sequence and one or more nucleotides comprising a first label;
b. amplifying the target DNA sequence to produce a nucleic acid amplification product; and
c. detecting the nucleic acid amplification product.

It is yet a further object to provide an ex vivo method of diagnosing a disease or disorder, preferably an infectious disease, in a subject, comprising determining the presence or absence of a target DNA sequence associated with the disease or disorder according to the invention in a sample which has been obtained from the subject, wherein the presence indicates a diagnosis of the disease or disorder in the subject.

Another object is to provide a method of detecting a nucleic acid amplification product comprising a target DNA sequence, the method comprising:
a. contacting the nucleic acid amplification product with a recombinase and an oligonucleotide probe comprising a nucleic acid sequence complementary to the target DNA sequence and a label, thereby forming in the nucleic acid amplification product a D-loop comprising triple-stranded DNA, the probe, and the recombinase; and
b. detecting the nucleic acid amplification product, comprising detecting the probe.

Another object is to provide a composition comprising:
dNTP monomers comprising a first label and an oligonucleotide probe comprising a second label; or
a first primer and a second primer for amplifying a target DNA sequence, wherein the second primer comprises a second label and is capable of hybridizing to a first strand of DNA of the target DNA sequence; and an oligonucleotide probe comprising a nucleic acid sequence complementary to the target DNA sequence and one or more nucleotides comprising a first label, preferably wherein the probe is capable of binding or hybridizing to the first strand or the second strand of DNA of the target DNA sequence, the second strand being complementary to the first strand.

Yet another object is to provide a lateral flow device, comprising a lateral flow strip, comprising:
a sample application zone;
a reagent zone downstream of the sample application zone and in fluid communication with the sample application zone, wherein the reagent zone comprises a non-immobilized first binding agent that binds to a first label;
a test zone downstream of the reagent zone and in fluid communication with the reagent zone, wherein the test zone comprises an immobilized second binding agent that binds to a second label; and
a control zone downstream of the test zone and in fluid communication with the test zone.

It is a further object to provide a kit of parts comprising:
a. optionally, a first primer and a second primer for amplifying a target DNA sequence;
b. a recombinase;
c. a DNA-dependent DNA polymerase;
d. dNTP monomers comprising a first label; and
e. an oligonucleotide probe comprising a nucleic acid sequence complementary to the target DNA sequence and a second label; and optionally
f. a lateral flow device or the device according to the invention.

It is yet a further object to provide a kit of parts comprising:
a. a first primer and a second primer for amplifying a target DNA sequence, wherein the second primer comprises a second label and hybridizes to a first strand of DNA of the target DNA sequence;
b. a recombinase;
c. a DNA-dependent DNA polymerase; and
d. an oligonucleotide probe comprising a nucleic acid sequence complementary to the target DNA sequence and one or more nucleotides comprising a first label, preferably wherein the probe hybridizes to the first strand or the second strand of DNA of the target DNA sequence, the second strand being complementary to the first strand; and optionally
e. a lateral flow device or the device according to the invention.

Yet another object is to provide a use of the device or kit of parts according to the invention for determining the presence or absence of a pathogen, preferably a human, animal, or plant pathogen, or a target DNA sequence associated with a disease or disorder or the onset thereof, or for determining the presence or absence of a target DNA sequence in a diagnostic sample, a veterinary sample, a pharmaceutical sample, a cosmetics sample, a food sample, a beverage sample, a feed sample, an agricultural sample, a soil sample, an air sample, a water sample, a waste sample, an effluent sample, or a sewage sample.

### SHORT DESCRIPTION OF THE FIGURES

FIG. 1 shows a schematic depiction of the first method of the invention.

FIG. 2 shows a schematic depiction of the second method of the invention.

FIG. 3 shows the effect of biotin-dCTP concentration on RPA yield.

FIG. 4 shows the effect of biotin-dUTP concentration on RPA yield.

FIG. 5 shows the effect of the effect of both biotin-dCTP and biotin-dUTP concentration on RPA yield.

FIG. 6 shows evidence of D-loop formation on agarose gel.

FIG. 7 shows evidence of D-loop formation in ELISA.

FIG. 8 shows evidence of D-loop formation in a lateral flow assay.

FIG. 9 shows effect of probe design on lateral flow assay.

FIG. 10 shows specificity of primer-probe and probe with and without ATP-gamma-S vs hapten-based strategy.

FIG. 11 shows that an RPA amplicon can be added directly into RecA-probe binding immobilized D loop detection with high reproducibility and specificity.

FIG. 12 shows that DrRecA does not require ATP or ATPγS for strand exchange.

FIG. 13 shows that bridge flocculation allows naked-eye detection of biotinylated DNA.

FIG. 14 shows stable D-loop formation by EcRecA and DrRecA.

### DETAILED DESCRIPTION OF THE INVENTION

The present invention relates to two methods having a similar concept, but wherein the first and second labels are present on different components. The second label is different from the first label.

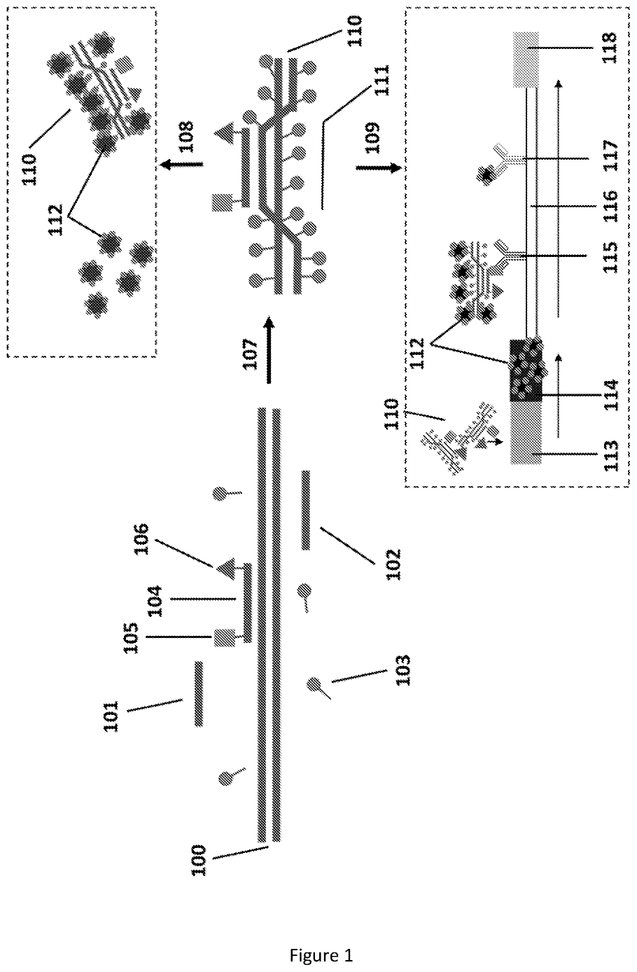

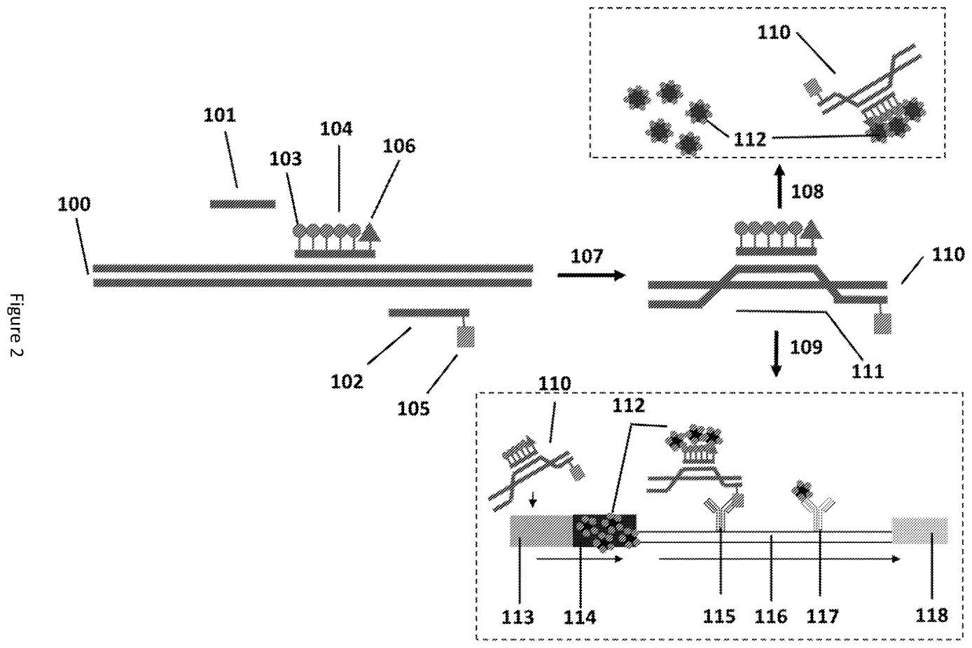

FIG. 1 shows an overview of the first method and FIG. 2 of the second method.

The present invention relates to a first method of determining the presence or absence of a target deoxyribonucleic acid (DNA) sequence in a sample, the method comprising:
a. contacting the sample with:
   a first primer and a second primer for amplifying the target DNA sequence;
   a recombinase;
   a DNA-dependent DNA polymerase;
   deoxynucleoside triphosphate (dNTP) monomers comprising a first label; and
   an oligonucleotide probe comprising a nucleic acid sequence complementary to the target DNA sequence and a second label;
b. amplifying the target DNA sequence to produce a nucleic acid amplification product; and
c. detecting the nucleic acid amplification product.

The present invention also relates to a second method of determining the presence or absence of a target deoxyribonucleic acid (DNA) sequence in a sample, the method comprising:
a. contacting the sample with:
   a first primer and a second primer for amplifying the target DNA sequence, wherein the second primer comprises a second label and hybridizes to a first strand of DNA of the target DNA sequence;
   a recombinase;
   a DNA-dependent DNA polymerase; and
   an oligonucleotide probe comprising a nucleic acid sequence complementary to the target DNA sequence and one or more nucleotides comprising a first label;
b. amplifying the target DNA sequence to produce a nucleic acid amplification product; and
c. detecting the nucleic acid amplification product.

Without being bound to theory, it is proposed that the recombinase and the probe according to the invention together enable the formation of a D-loop in the nucleic acid amplification product at a sequence complementary to the probe, thereby forming triple-stranded DNA. ‘D-loop’ herein is understood to mean a DNA structure where the two strands of a double-stranded DNA molecule are separated by a certain length and held apart by a third strand of DNA, optionally comprising the recombinase. This is also known as a single D-loop, which is different from a double D-loop where two strands of a double-stranded DNA molecule are separated by a certain length and held apart by two additional strands of DNA, thereby forming quadruple-stranded DNA. Thus, the D-loop according to the invention comprises triple-stranded DNA. Recombinase may still be bound in the D-loop, preferably by a means for modulating binding of the recombinase to the DNA, or it may be dissociated. If the latter occurs, the probe should still be bound in the D-loop, preferably by a means for modulating binding of the labeled probe to the DNA. A D-loop complex thought to be formed by the method according to the invention, comprising the probe and the amplified target DNA sequence, and optionally the recombinase, has until now not been shown to be possible to be used for detection of nucleic acid amplification products. Hence, the inventors have surprisingly shown that it can be used as such and applied it for reliable detection of nucleic acid amplification products. According to the invention, the nucleic acid amplification product preferably comprises triple-stranded DNA, preferably DNA comprising a D-loop.

The inventors have shown that a nucleic acid amplification product comprising a D-loop comprising triple-stranded DNA, the probe, and the recombinase is sufficiently stable to be detected by detecting the probe. Thus, step c of detecting the nucleic acid amplification product of the first or second method according to the invention preferably comprises the nucleic acid amplification product comprising a D-loop, the D-loop comprising triple-stranded DNA, the probe, and the recombinase, wherein detecting the nucleic acid amplification product comprises detecting, preferably binding, the probe.

Preferably, contacting the sample with the recombinase and/or the probe may be performed before, during, or after step b of amplifying the target DNA sequence.

Herein, the terms ‘triple-stranded DNA’ and ‘DNA triplex’ are used interchangeably. It is to be understood that herein triple-stranded DNA refers to an intermolecular DNA triplex and not to an intramolecular DNA triplex. An intermolecular triplex is formed between a (triplex-forming) oligonucleotide, such as an oligonucleotide probe according to the invention, and a target DNA sequence on double stranded DNA (dsDNA). An intramolecular triplex is for example formed when the third strand is part of a single strand which also contains the dsDNA.

The term ‘nuclease-free’ as used in herein is understood to mean that no nuclease is used for formation of a signal (or pre-cursor thereof) for detection, e.g. the formation of a signal is facilitated without a nuclease. Similarly, the term ‘protease-free’ as used in herein is understood to mean that no protease is used for formation of a signal (or pre-cursor thereof) for detection, e.g. the formation of a signal is facilitated without a protease. For example, when performing the method according to the invention no nuclease or protease is used during any step of the method. Hence, preferably no nucleases and/or proteases are present in the kits and compositions according to the invention. As disclosed above and herein, the present invention dispenses with the need of enzymatic post-processing of a nucleic acid amplification product (e.g. via nuclease or protease) for detection of triple-stranded DNA complexes, while only using a single-stranded labelled probe and a recombinase.

The oligonucleotide probe according to the invention is preferably single-stranded, preferably comprising single-stranded DNA. The oligonucleotide probe comprising a nucleic acid sequence complementary to the target DNA sequence and a second label according to the invention is preferably single-stranded, preferably comprising single-stranded DNA. The oligonucleotide probe comprising a nucleic acid sequence complementary to the target DNA sequence and one or more nucleotides comprising a first label is preferably single-stranded, preferably comprising single-stranded DNA. The single-stranded nature of the oligonucleotide probe enables, together with a recombinase and a target DNA sequence, the formation of a D-loop comprising triple-stranded DNA according to the invention.

The oligonucleotide probe comprising a nucleic acid sequence complementary to the target DNA sequence and a second label according to the invention may comprise a partial nucleic acid sequence or the full nucleic acid sequence of one of the primers used for amplification.

The oligonucleotide probe comprising a nucleic acid sequence complementary to the target DNA sequence and one or more nucleotides comprising a first label according to the invention may comprise a partial nucleic acid sequence or the full nucleic acid sequence of the first primer used for amplification. The probe comprising a partial nucleic acid sequence or the full nucleic acid sequence of one of the primers may facilitate the formation of a D-loop in a nucleic acid amplification product. Preferably, the partial nucleic acid sequence comprises at least 1%, 5%, 10%, 15%, 20%, 25%, 30%, 40%, 50%, 60%, 70%, 80%, or 90% of the full nucleic acid sequence of one of the primers used for amplification or the first primer used for amplification. Preferably, the partial nucleic acid sequence comprises at least about 1%, about 5%, about 10%, about 15%, about 20%, about 25%, about 30%, about 40%, about 50%, about 60%, about 70%, about 80%, or about 90% of the full nucleic acid sequence of one of the primers used for amplification or the first primer used for amplification. Preferably, the probe comprising a partial nucleic acid sequence of one of the primers or the first primer used for amplification has a length of at least 25 nucleotides, more preferably of at least 26, at least 27, at least 28, at least 29, at least 30, at least 31, at least 32, at least 33, at least 34, at least 35, at least 40, at least 45, at least 50, at least 55, or at least 60 nucleotides. Preferably, the probe comprising a partial nucleic acid sequence of one of the primers or the first primer used for amplification has a length of from 25 to 100 nucleotides, more preferably of from 26 to 100, of from 26 to 95, of from 26 to 90, of from 26 to 85, of from 26 to 80, of from 26 to 75, of from 26 to 70, of from 26 to 65, of from 26 to 60, of from 27 to 100, of from 27 to 95, of from 27 to 90, of from 27 to 85, of from 27 to 80, of from 27 to 75, of from 27 to 70, of from 27 to 65, of from 28 to 100, of from 28 to 95, of from 28 to 90, of from 28 to 85, of from 28 to 80, of from 28 to 75, of from 28 to 70, of from 28 to 65, of from 30 to 100, of from 30 to 95, of from 30 to 90, of from 30 to 85, of from 30 to 80, of from 30 to 75, of from 30 to 70, of from 30 to 65, of from 32 to 95, of from 32 to 90, of from 32 to 85, of from 32 to 80, of from 32 to 75, of from 32 to 70, of from 32 to 65, of from 32 to 60, of from 35 to 100, of from 35 to 95, of from 35 to 90, of from 35 to 85, of from 35 to 80, of from 35 to 75, of from 35 to 70, of from 35 to 65, of from 38 to 100, of from 38 to 95, of from 38 to 90, of from 38 to 85, of from 38 to 80, of from 38 to 75, of from 38 to 70, of from 38 to 65, of from 40 to 100, of from 40 to 95, of from 40 to 90, of from 40 to 85, of from 40 to 80, of from 40 to 75, of from 40 to 70, or of from 40 to 65 nucleotides.

Preferably, the probe comprising a partial nucleic acid sequence of one of the primers or the first primer used for amplification has a length of from 25 to 100 nucleotides, more preferably of from 26, 27, 28, 29, 30, 31, 32, 33, 34, or 35 to 50, 51, 52, 53, 54, 55, 56, 57, 58, 59, 60, 65, 70, 75, 80, 85, 90, 95, or 100 nucleotides.

In order to prevent the formation of complexes between the probe and a primer, the probe should not comprise a sequence that is (substantially) fully complementary to one of the other primers present in the sample. When the nucleic acid amplification product has been isolated by for example DNA purification, no primers should be present, and the probe may then comprise a sequence that is fully complementary or partially complementary to one of the other primers present in the sample.

The invention also relates to a first composition comprising dNTP monomers comprising a first label and an oligonucleotide probe comprising a second label. Preferably, the composition is for amplification of a nucleic acid, preferably wherein the amplification is recombinase polymerase amplification (RPA). Preferably, the composition comprises a recombinase. The first composition according to the invention may further comprise features of the first method of the invention or features of the second method of the invention in so far possible.

The invention further relates to a second composition comprising a first primer and a second primer for amplifying a target DNA sequence, wherein the second primer comprises a second label and is capable of hybridizing to a first strand of DNA of the target DNA sequence; and an oligonucleotide probe comprising a nucleic acid sequence complementary to the target DNA sequence and one or more nucleotides comprising a first label, preferably wherein the probe is capable of binding or hybridizing to the first strand or the second strand of DNA of the target DNA sequence, the second strand being complementary to the first strand. Preferably, the composition is for amplification of a nucleic acid, preferably wherein the amplification is recombinase polymerase amplification (RPA). Preferably, the composition comprises a recombinase. The second composition according to the invention may further comprise features of the second method of the invention or features of the first method of the invention in so far possible.

Preferably, the composition according to the invention is nuclease-free or protease-free. Preferably, the composition is nuclease-free and protease-free.

Methods to perform various kinds of suitable nucleic acid amplification reactions, thereby amplify the target DNA sequence according to the invention, are well-known in the art. Various reviews are available that refer to such methods, for example Zhu et al. (BioTechniques, 2020, vol. 69, no. 4) and Glökler et al. (Critical Reviews in Biochemistry and Molecular Biology, 2021, vol. 56, issue 6, p 543-586). For example, for RPA, WO2010141940A1 and Piepenburg et al. describe suitable methods and reagents. These well-known methods include how and when to contact the sample according to the invention with primers, dNTPs, enzymes, probes, and other reaction components required for the amplification. For example, a first primer and a second primer for amplifying the target DNA sequence according to the invention include a sense and an antisense primer that comprise sequences that are complementary to the target DNA sequence, which are suitable for forming a double-stranded DNA molecule comprising the target DNA sequence during the amplification reaction. In addition, various methods how to detect the amplification product according to the invention after amplification or during amplification, also termed real-time detection, such as real-time PCR or real-time RPA, are well-known in the art.

According to the method of the invention, contacting the sample with the recombinase and/or the probe may be performed before, during, or after step b of amplifying the target DNA sequence. Preferably, contacting the sample with the recombinase may be performed before, during, or after step b of amplifying the target DNA sequence. Preferably, contacting the sample with the probe may be performed before, during, or after step b of amplifying the target DNA sequence. For instance, if an RPA reaction is to be performed for step b, the sample will have to be contacted with the recombinase before or during step b, but additional recombinase may be added to the sample, thereby contacting, after the amplification reaction. Additional recombinase and/or probe may be added to the sample after any type of amplification reaction. The sample may also be contacted with the recombinase for a first time after step b of amplifying the target DNA sequence if an amplification reaction different from an RPA reaction has been performed.

Detecting the nucleic acid amplification product and thereby the target DNA sequence is qualitative (i.e. the presence or absence), but in addition may also be quantitative (i.e. the amount of product). Preferably, detecting the nucleic acid amplification product comprises detecting, preferably binding, the probe.

A ‘target DNA sequence’ is herein understood to mean a molecule comprising the target DNA sequence, preferably a molecule mostly composed of DNA such as a DNA molecule. Preferably, the DNA molecule comprises or consists of double-stranded DNA. A DNA sequence, e.g. an oligonucleotide or a target DNA sequence, comprising a dNTP monomer herein is understood to mean that the dNTP monomer is incorporated into the DNA sequence as a nucleotide.

‘Hybridization’ herein is understood to mean the binding of a single-stranded nucleic acid molecule, preferably DNA or RNA, to a complementary single-stranded nucleic acid sequence. The binding may occur despite one or more mismatches between the sequence of the single-stranded nucleic acid molecule and the complementary single-stranded nucleic acid sequence. Binding of a primer or a probe according to the present invention preferably occurs via hybridization. With regard to the mismatches, a probe according to the invention is expected to have the same characteristics as primers used in RPA reactions, which is for example disclosed in Daher et al. (Molecular and Cellular Probes, 2015, vol. 29, issue 2, p 116-121) and Liu et al. (World Journal of Microbiology and Biotechnology, 2019, 35, Article number: 95). A person skilled in the art can design suitable probes for the invention. ‘Contacting’ herein is understood to mean any suitable means for delivering, or exposing, a sample to the compounds, e.g. DNA molecules, dNTPs, or proteins, described herein so as to permit physical and/or chemical interaction between the sample and the compound. Suitably, the interaction may be between the target DNA sequence in the sample on one hand and the primers, the probe, the recombinase, and the DNA-dependent polymerase on the other hand. In some instances, the term ‘contacting’ refers to providing the compounds described herein (e.g., suspended in a solution) directly to the sample. Alternatively, in some instances the term ‘contacting’ refers to providing the sample to the compounds described herein (e.g., attached to a solid support). The term ‘contacting’ can further comprise mixing the sample with the compounds by any means known in the art (e.g., vortexing, pipetting, and/or agitating). In some instances, the term ‘contacting’ can further comprise incubating the sample together with the compounds for a sufficient amount of time, e.g., to allow binding of the target DNA sequence in the sample to the compounds. The contact time can be of any length, depending on the binding affinities and/or concentrations of the compounds and/or molecules comprising the target DNA sequence in the sample, concentrations of a detection reagent, and/or incubation condition (e.g., temperature). In some embodiments, the contact time between the sample and the compounds can be at least about 30 seconds, at least about 1 minute, at least about 3 minutes, at least about 5 minutes, at least about 10 minutes, at least about 15 minutes, at least about 25 minutes, at least about 30 minutes, at least about 1 hour, at least about 2 hours, at least about 3 hours, at least about 4 hours, at least about 6 hours, at least about 8 hours, at least about 10 hours, at least about 12 hours, at least about 24 hours, at least about 48 hours or longer. Alternatively, for example where essential reaction components have been consumed, a longer incubation time may not be beneficial. One of skill in the art can adjust the contact time accordingly.

In the first method of the invention, dNTP monomers comprising a first label are incorporated into the nucleic acid amplification product during amplification, thereby becoming nucleotides, and provide a first label for detection, while the oligonucleotide probe comprising a second label, when bound to the amplification product, provides a second label for detection.

According to the first method of the invention, detecting the nucleic acid amplification product in step c preferably comprises binding one or more first labels of the dNTP monomers of the nucleic acid amplification product to a first binding agent and binding the second label of the probe bound to the nucleic acid amplification product to a second binding agent, preferably wherein the first label does not bind to the second binding agent and the second label does not bind to the first binding agent. Preferably, at least two first labels are bound, preferably each by an individual first binding agent or by at least two first binding agents. Preferably, at least three, four, five, six, seven, eight, nine, or ten first labels are bound, preferably each by an individual first binding agent. Preferably, at least three, four, five, six, seven, eight, nine, or ten first labels are bound, preferably by at least three, four, five, six, seven, eight, nine, or ten first binding agents.

Preferably, the first binding agent is capable of binding to the first label. Preferably, the second binding agent is capable of binding to the second label. Preferably, the first binding agent binds specifically to the first label. Preferably, the second binding agent binds specifically to the second label. Preferably, the first binding agent is attached to a solid support.

‘Capable of binding’ or ‘binding’ regarding binding agents, e.g. antibodies, herein is understood to mean binding agents capable of binding to defined labels, e.g. proteins or polysaccharides, under the usual experimental conditions of biological binding assays. In the context of the invention, a binding agent which binds or is capable of binding to a defined label (e.g. biotin or digoxigenin) forms or undergoes a physical association with it, in an amount and for a time to sufficiently allow detection of the binding agent-label complex. Preferably, a binding agent is capable of binding specifically to a specific label. In the context of the invention, a binding agent which binds specifically (or an antibody that specifically binds) to a defined label (e.g. biotin or digoxigenin) forms or undergoes a physical association with it, in an amount and for a time to sufficiently allow detection of the binding agent-label complex. By ‘specifically’ or ‘specific for’, it is meant that the binding agent has a higher affinity for the defined label than for other labels, such as for instance other labels contained in the sample. In the context of the invention, the term ‘affinity’ when referring to binding agents, e.g. antibodies or streptavidin, designate the strength with which the binding agent binds to a defined label, or a part thereof, and is measured by the affinity constant between the antibody and the label (defined as 1/KD, wherein KD is the dissociation constant as classically defined) measurement of the reaction rate constants can be used to define an equilibrium or affinity constant (1/KD). The affinity of a binding agent for its target is thus inversely correlated to the dissociation constant, i.e. the smaller the KD value the greater the affinity of the binding agent for its target. For example, the binding agent can have an affinity for the defined label of at least about 1.5-fold, 2-fold, 2.5-fold, 3-fold, 4-fold, 5-fold, or 10-fold, or higher than for other labels in the sample. For example, the binding agent can have an affinity for the defined label of at least about 1.5-fold, about 2-fold, about 2.5-fold, about 3-fold, about 4-fold, about 5-fold, or about 10-fold, or higher than for other labels in the sample. Such affinity or degree of specificity can be determined by a variety of routine procedures, including competitive binding assays.

According to the first method of the invention, contacting the sample with dNTP monomers comprising a first label preferably comprises contacting the sample with a mixture comprising of from 30 to 70% deoxycytidine triphosphate (dCTP) comprising a first label, of from 30 to 70% deoxyadenosine (dATP) comprising a first label, of from 30 to 70% deoxyguanosine (dGTP) comprising a first label, of from 30 to 70% deoxythymidine (dTTP) comprising a first label, and/or of from 30 to 70% deoxyuridine triphosphate (dUTP) comprising a first label, each based on the total number of moles dNTP of its base type. Preferably, the mixture comprises of from 30 to 70% dCTP comprising a first label and/or of from 30 to 70% dUTP comprising a first label.

According to the first method of the invention, contacting the sample with dNTP monomers comprising a first label preferably comprises contacting the sample with a mixture comprising of from 30 to 70% biotin-labeled dCTP, of from 30 to 70% biotin-labeled dATP, of from 30 to 70% biotin-labeled dGTP, of from 30 to 70% dTTP, and/or of from 30 to 70% biotin-labeled dUTP, each based on the total number of moles dNTP of its base type, wherein the first label is biotin. Preferably, the mixture comprises of from 30 to 70% biotin-labeled dCTP and/or of from 30 to 70% biotin-labeled dUTP. The inventors have found that the above ratios of biotin-labeled to non-labeled dNTPs result in improved detection of the amplification product.

According to the first method of the invention, contacting the sample with dNTP monomers comprising a first label preferably comprises contacting the sample with a mixture comprising of from about 30 to about 70% deoxycytidine triphosphate (dCTP) comprising a first label, of from about 30 to about 70% deoxyadenosine (dATP) comprising a first label, of from about 30 to about 70% deoxyguanosine (dGTP) comprising a first label, of from about 30 to about 70% deoxythymidine (dTTP) comprising a first label, and/or of from about 30 to about 70% deoxyuridine triphosphate (dUTP) comprising a first label, each based on the total number of moles dNTP of its base type. Preferably, the mixture comprises of from about 30 to about 70% dCTP comprising a first label and/or of from about 30 to about 70% dUTP comprising a first label.

According to the first method of the invention, contacting the sample with dNTP monomers comprising a first label preferably comprises contacting the sample with a mixture comprising of from about 30 to about 70% biotin-labeled dCTP, of from about 30 to about 70% biotin-labeled dATP, of from about 30 to about 70% biotin-labeled dGTP, of from about 30 to about 70% dTTP, and/or of from about 30 to about 70% biotin-labeled dUTP, each based on the total number of moles dNTP of its base type, wherein the first label is biotin. Preferably, the mixture comprises of from about 30 to about 70% biotin-labeled dCTP and/or of from about 30 to about 70% biotin-labeled dUTP. The inventors have found that the above ratios of biotin-labeled to non-labeled dNTPs result in improved detection of the amplification product.

‘Base type’ herein is understood to mean a type of any of the nitrogenous bases; adenine, guanine, thymine, uracil, and cytosine, that are comprised by their respective deoxy(ribose)nucleoside.

For example, the mixture according to the invention may comprise 30% dCTP and 70% biotin-labeled dCTP. In another example, the mixture may comprise 40% dCTP, 60% biotin-labeled dCTP, 50% dUTP, and 50% biotin-labeled dUTP, totaling up to 100% of the base type cytosine and 100% of the base type uracil. Hence, the mixture comprises a total of 100% of a respective dNTP of which a defined percentage comprises a first label and the remaining percentage does not comprise the first label. It is understood that the defined mixtures are present for the nucleic acid amplification reaction.

Preferably, the mixture according to the invention comprises of from about 30 to about 65%, of from about 30 to about 60%, of from about 30 to about 55%, of from about 30 to about 50%, of from about 30 to about 45%, of from about 35 to about 70%, of from about 35 to about 65%, of from about 35 to about 60%, of from about 35 to about 55%, of from about 35 to about 45%, of from about 40 to about 70%, of from about 40 to about 65%, of from about 40 to about 60%, of from about 40 to about 55%, of from about 40 to about 50%, of from about 45 to about 70%, of from about 45 to about 65%, of from about 45 to about 60%, of from about 45 to about 55%, of from about 50 to about 70%, of from about 50 to about 65%, or of from about 50 to about 60% dCTP comprising a first label, dATP comprising a first label, dGTP comprising a first label, dTTP comprising a first label and/or dUTP comprising a first label, preferably dCTP comprising a first label and/or dUTP comprising a first label, each based on the total number of moles dNTP of its base type. Preferably, the mixture according to the invention comprises of from about 35, about 36, about 37, about 38, about 39, about 40, about 41, about 42, about 43, about 44, or about 45 to about 55, about 56, about 57, about 58, about 59, about 60, about 61, about 62, about 63, about 64, or about 65% dCTP comprising a first label, dATP comprising a first label, dGTP comprising a first label, dTTP comprising a first label and/or dUTP comprising a first label, preferably dCTP comprising a first label and/or dUTP comprising a first label, each based on the total number of moles dNTP of its base type. Preferably, the mixture comprises of from about 35 to about 60%, of from about 35 to about 55%, of from about 35 to about 50%, of from about 40 to about 55%, of from about 40 to about 60%, of from about 45 to about 55%, or of from about 45 to about 60% dCTP comprising a first label based on the total number of moles dCTP. Preferably, the mixture comprises of from about 35 to about 60%, of from about 35 to about 55%, of from about 35 to about 50%, of from about 40 to about 60%, of from about 40 to about 55%, of from about 45 to about 55%, or of from about 45 to about 60% dUTP comprising a first label based on the total number of moles dUTP. Preferably, the mixture comprises of from about 35 to about 45% dCTP comprising a first label and of from about 55 to about 65% dUTP comprising a first label, based on the total number of moles dNTP of its base type.

Preferably, the mixture according to the invention comprises of from 30 to 65%, of from 30 to 60%, of from 30 to 55%, of from 30 to 50%, of from 30 to 45%, of from 35 to 70%, of from 35 to 65%, of from 35 to 60%, of from 35 to 55%, of from 35 to 45%, of from 40 to 70%, of from 40 to 65%, of from 40 to 60%, of from 40 to 55%, of from 40 to 50%, of from 45 to 70%, of from 45 to 65%, of from 45 to 60%, of from 45 to 55%, of from 50 to 70%, of from 50 to 65%, or of from 50 to 60% dCTP comprising a first label, dATP comprising a first label, dGTP comprising a first label, dTTP comprising a first label and/or dUTP comprising a first label, preferably dCTP comprising a first label and/or dUTP comprising a first label, each based on the total number of moles dNTP of its base type. Preferably, the mixture according to the invention comprises of from 35, 36, 37, 38, 39, 40, 41, 42, 43, 44, or 45 to 55, 56, 57, 58, 59, 60, 61, 62, 63, 64, or 65% dCTP comprising a first label, dATP comprising a first label, dGTP comprising a first label, dTTP comprising a first label and/or dUTP comprising a first label, preferably dCTP comprising a first label and/or dUTP comprising a first label, each based on the total number of moles dNTP of its base type. Preferably, the mixture comprises of from 35 to 60%, of from 35 to 55%, of from 35 to 50%, of from 40 to 55%, of from 40 to 60%, of from 45 to 55%, or of from 45 to 60% dCTP comprising a first label based on the total number of moles dCTP. Preferably, the mixture comprises of from 35 to 60%, of from 35 to 55%, of from 35 to 50%, of from 40 to 60%, of from 40 to 55%, of from 45 to 55%, or of from 45 to 60% dUTP comprising a first label based on the total number of moles dUTP. Preferably, the mixture comprises of from 35 to 45% dCTP comprising a first label and of from 55 to 65% dUTP comprising a first label, based on the total number of moles dNTP of its base type.

Preferably, the mixture according to the invention comprises of from about 30 to about 65%, of from about 30 to about 60%, of from about 30 to about 55%, of from about 30 to about 50%, of from about 30 to about 45%, of from about 35 to about 70%, of from about 35 to about 65%, of from about 35 to about 60%, of from about 35 to about 55%, of from about 35 to about 45%, of from about 40 to about 70%, of from about 40 to about 65%, of from about 40 to about 60%, of from about 40 to about 55%, of from about 40 to about 50%, of from about 45 to about 70%, of from about 45 to about 65%, of from about 45 to about 60%, of from about 45 to about 55%, of from about 50 to about 70%, of from about 50 to about 65%, or of from about 50 to about 60% biotin-labeled dCTP, biotin-labeled dATP, biotin-labeled dGTP, biotin-labeled dTTP and/or biotin-labeled dUTP, preferably dCTP and/or dUTP comprising a first label, each based on the total number of moles dNTP of its base type. Preferably, the mixture according to the invention comprises of from about 35, about 36, about 37, about 38, about 39, about 40, about 41, about 42, about 43, about 44, or about 45 to about 55, about 56, about 57, about 58, about 59, about 60, about 61, about 62, about 63, about 64, or about 65% biotin-labeled dCTP, biotin-labeled dATP, biotin-labeled dGTP, biotin-labeled dTTP and/or biotin-labeled dUTP, preferably dCTP and/or dUTP comprising a first label, each based on the total number of moles dNTP of its base type. Preferably, the mixture comprises about 50% biotin-labeled dCTP, about 50% biotin-labeled dUTP, or about 40% biotin-labeled dCTP and 60% biotin-labeled dUTP. Preferably, the mixture comprises of from about 45% to about 55% biotin-labeled dCTP, of from about 45% to about 55% biotin-labeled dUTP, or of from about 35% to about 45% biotin-labeled dCTP and of from about 55% to about 65% biotin-labeled dUTP, each based on the total number of moles dNTP of its base type.

According to the first method of the invention, contacting the sample with dNTP monomers comprising a first label preferably comprises contacting the sample with a mixture comprising up to about 55% dCTP comprising a first label, up to about 55% dATP comprising a first label, up to about 55% dGTP comprising a first label, up to about 55% dTTP comprising a first label, and/or up to about 55% dUTP comprising a first label, each based on the total number of moles dNTP of its base type. Preferably, the mixture comprises up to about 55% dCTP comprising a first label and/or up to about 55% deoxyuridine dUTP comprising a first label. Preferably, the mixture comprises up to about 50%, up to about 45%, up to about 40%, up to about 35%, up to about 30%, up to about 25%, up to about 20%, up to about 15%, up to about 10%, or up to about 5% dCTP comprising a first label, dATP comprising a first label, dGTP comprising a first label, dTTP comprising a first label and/or dUTP comprising a first label, preferably dCTP comprising a first label and/or dUTP comprising a first label, each based on the total number of moles dNTP of its base type

According to the first method of the invention, contacting the sample with dNTP monomers comprising a first label preferably comprises contacting the sample with a mixture comprising up to about 55% biotin-labeled dCTP, up to about 55% biotin-labeled dATP, up to about 55% biotin-labeled dGTP, up to about 55% dTTP, and/or up to about 55% biotin-labeled dUTP, each based on the total number of moles dNTP of its base type, wherein the first label is biotin. Preferably, the mixture comprises up to about 55% biotin-labeled dCTP and/or up to about 55% biotin-labeled dUTP. Preferably, the mixture comprises up to about 50%, up to about 45%, up to about 40%, up to about 35%, up to about 30%, up to about 25%, up to about 20%, up to about 15%, up to about 10%, or up to about 5% biotin-labeled dCTP, biotin-labeled dATP, biotin-labeled dGTP, biotin-labeled dTTP and/or biotin-labeled dUTP, preferably dCTP and/or dUTP comprising a first label, each based on the total number of moles dNTP of its base type.

Preferably, the mixture according to the invention comprises of from 30 to 65%, of from 30 to 60%, of from 30 to 55%, of from 30 to 50%, of from 30 to 45%, of from 35 to 70%, of from 35 to 65%, of from 35 to 60%, of from 35 to 55%, of from 35 to 45%, of from 40 to 70%, of from 40 to 65%, of from 40 to 60%, of from 40 to 55%, of from 40 to 50%, of from 45 to 70%, of from 45 to 65%, of from 45 to 60%, of from 45 to 55%, of from 50 to 70%, of from 50 to 65%, or of from 50 to 60% biotin-labeled dCTP, biotin-labeled dATP, biotin-labeled dGTP, biotin-labeled dTTP and/or biotin-labeled dUTP, preferably dCTP and/or dUTP comprising a first label, each based on the total number of moles dNTP of its base type. Preferably, the mixture according to the invention comprises of from 35, 36, 37, 38, 39, 40, 41, 42, 43, 44, or 45 to 55, 56, 57, 58, 59, 60, 61, 62, 63, 64, or 65% biotin-labeled dCTP, biotin-labeled dATP, biotin-labeled dGTP, biotin-labeled dTTP and/or biotin-labeled dUTP, preferably dCTP and/or dUTP comprising a first label, each based on the total number of moles dNTP of its base type. Preferably, the mixture comprises of from 45% to 55% biotin-labeled dCTP, of from 45% to 55% biotin-labeled dUTP, or of from 35% to 45% biotin-labeled dCTP and of from 55% to 65% biotin-labeled dUTP, each based on the total number of moles dNTP of its base type.

According to the first method of the invention, contacting the sample with dNTP monomers comprising a first label preferably comprises contacting the sample with a mixture comprising up to 55% dCTP comprising a first label, up to 55% dATP comprising a first label, up to 55% dGTP comprising a first label, up to 55% dTTP comprising a first label, and/or up to 55% dUTP comprising a first label, each based on the total number of moles dNTP of its base type. Preferably, the mixture comprises up to 55% dCTP comprising a first label and/or up to 55% deoxyuridine dUTP comprising a first label. Preferably, the mixture comprises up to 50%, up to 45%, up to 40%, up to 35%, up to 30%, up to 25%, up to 20%, up to 15%, up to 10%, or up to 5% dCTP comprising a first label, dATP comprising a first label, dGTP comprising a first label, dTTP comprising a first label and/or dUTP comprising a first label, preferably dCTP comprising a first label and/or dUTP comprising a first label, each based on the total number of moles dNTP of its base type

According to the first method of the invention, contacting the sample with dNTP monomers comprising a first label preferably comprises contacting the sample with a mixture comprising up to 55% biotin-labeled dCTP, up to 55% biotin-labeled dATP, up to 55% biotin-labeled dGTP, up to 55% dTTP, and/or up to 55% biotin-labeled dUTP, each based on the total number of moles dNTP of its base type, wherein the first label is biotin. Preferably, the mixture comprises up to 55% biotin-labeled dCTP and/or up to 55% biotin-labeled dUTP. Preferably, the mixture comprises up to 50%, up to 45%, up to 40%, up to 35%, up to 30%, up to 25%, up to 20%, up to 15%, up to 10%, or up to 5% biotin-labeled dCTP, biotin-labeled dATP, biotin-labeled dGTP, biotin-labeled dTTP and/or biotin-labeled dUTP, preferably dCTP and/or dUTP comprising a first label, each based on the total number of moles dNTP of its base type.

In the second method of the invention, one or more dNTPs comprising a first label have been incorporated into the oligonucleotide probe and are thereby also known as nucleotides. These provide a first label for detection when the probe is bound to the target DNA sequence. The second primer comprises a second label and provides a second label for detection when the primer is hybridized to the target DNA sequence.

According to the second method of the invention, the second primer preferably hybridizes to a first strand of DNA of the target DNA sequence during step b of amplifying the target DNA sequence to produce a nucleic acid amplification product.

According to the second method of the invention, the probe preferably hybridizes to the first strand or the second strand of DNA of the target DNA sequence, the second strand being complementary to the first strand.

According to the second method of the invention, detecting the nucleic acid amplification product in step c preferably comprises binding one or more first labels of the nucleotides of the probe to a first binding agent and binding the second label of the second primer bound to the nucleic acid amplification product to a second binding agent, preferably wherein the first label does not bind to the second binding agent and the second label does not bind to the first binding agent. Preferably, the first binding agent is capable of binding to the first label. Preferably, the second binding agent is capable of binding to the second label. Preferably, the first binding agent binds specifically to the first label. Preferably, the second binding agent binds specifically to the second label. Preferably, the second binding agent is attached to a solid support.

According to the second method of the invention, the probe preferably comprises at least two nucleotides comprising a first label. Preferably, the probe comprises at least three, four, five, six, seven, eight, nine, or ten nucleotides comprising a first label.

According to the second method of the invention, the probe preferably comprises of from about 10, about 20, about 30, or about 40 to about 60, about 70, about 80, or about 90% nucleotides comprising a first label based on the total number of nucleotides of the probe. According to the second method of the invention, the probe preferably comprises of from about 10 to about 90%, from about 10 to about 80%, from about 10 to about 70%, from about 10 to about 60%, from about 10 to about 50%, from about 10 to about 40%, from about 10 to about 30%, from about 10 to about 20%, from about 20 to about 90%, from about 20 to about 80%, from about 20 to about 70%, from about 20 to about 60%, from about 20 to about 50%, from about 20 to about 40%, from about 20 to about 30%, from about 30 to about 90%, from about 30 to about 80%, from about 30 to about 70%, from about 30 to about 60%, from about 30 to about 50%, from about 30 to about 40%, from about 40 to about 90%, from about 40 to about 80%, from about 40 to about 70%, from about 40 to about 60%, from about 40 to about 50%, from about 50 to about 90%, from about 50 to about 80%, from about 50 to about 70%, from about 50 to about 60%, from about 60 to about 90%, from about 60 to about 80%, from about 60 to about 70%, from about 70 to about 90%, from about 70 to about 80%, or from about 80 to about 90% nucleotides comprising a first label based on the total number of nucleotides of the probe.

According to the second method of the invention, the probe preferably comprises of from 10, 20, 30, or 40 to 60, 70, 80, or 90% nucleotides comprising a first label based on the total number of nucleotides of the probe. According to the second method of the invention, the probe preferably comprises of from 10 to 90%, from 10 to 80%, from 10 to 70%, from 10 to 60%, from 10 to 50%, from 10 to 40%, from 10 to 30%, from 10 to 20%, from 20 to 90%, from 20 to 80%, from 20 to 70%, from 20 to 60%, from 20 to 50%, from 20 to 40%, from 20 to 30%, from 30 to 90%, from 30 to 80%, from 30 to 70%, from 30 to 60%, from 30 to 50%, from 30 to 40%, from 40 to 90%, from 40 to 80%, from 40 to 70%, from 40 to 60%, from 40 to 50%, from 50 to 90%, from 50 to 80%, from 50 to 70%, from 50 to 60%, from 60 to 90%, from 60 to 80%, from 60 to 70%, from 70 to 90%, from 70 to 80%, or from 80 to 90% nucleotides comprising a first label based on the total number of nucleotides of the probe.

According to the invention, amplifying the target DNA sequence to produce a nucleic acid amplification product preferably comprises performing a PCR reaction or an isothermal amplification reaction. Preferably, the target DNA sequence is amplified by performing a PCR reaction or an isothermal amplification reaction. Preferably, the isothermal amplification reaction is a recombinase polymerase amplification (RPA) reaction. According to the invention, the nucleic acid amplification product preferably comprises double-stranded DNA.

The sample according to the invention may be contacted with the probe and the recombinase after amplifying the target DNA sequence according to the invention, preferably by performing a nucleic acid amplification reaction other than an RPA reaction. The sample according to the invention may be contacted with the recombinase after amplifying the target DNA sequence according to the invention, preferably by performing a nucleic acid amplification reaction other than an RPA reaction. As such, the recombinase facilitates binding of the probe to the target DNA sequence only after the nucleic acid amplification. Preferably, after amplifying the target DNA sequence and before detecting the amplification product, an additional step of isolating the nucleic acid amplification product and/or removing the polymerase is performed followed by contacting the nucleic acid amplification product with the probe and the recombinase. The step of isolating the amplification product and/or removing the polymerase minimizes amongst others the detection of potential off-target or ab initio amplification products after addition of the probe and the recombinase. Alternatively, the sample may be contacted with the probe after amplifying the target DNA sequence according to the invention and after the sample has been contacted with the recombinase before or during amplification, preferably by performing a nucleic acid amplification reaction other than an RPA reaction. Also, according to the invention, the sample may be contacted with the probe during step b of amplifying the target DNA sequence or after step b, preferably wherein the sample is not contacted with the probe prior to step b, preferably by performing a nucleic acid amplification reaction other than an RPA reaction. According to the invention, the sample may be contacted with the probe after step b and the sample is contacted with an additional amount of recombinase, preferably wherein the sample is not contacted with the probe prior to step b.

According to the invention, the probe preferably comprises a polymerase blocking group, preferably a DNA-dependent DNA polymerase blocking group. A blocking group prevents polymerase extension of a primer or the probe, which could also function as a primer, according to the invention, in particular during RPA. If no polymerase or essential components for substantial function of the polymerase are present, a polymerase blocking group on the probe is not required. Further, a polymerase blocking group on the probe is not required when a polymerase is present under conditions where it does not function or has minimal function. For example, a single Taq polymerase, used for PCR reactions, incorporates about 60 nucleotides/s at 70 °C, but about 0.25 nucleotides/s at 22 °C. RPA may be performed at from 20 to 50 °C depending on the enzymes used, although research in the field for use at a greater temperature range is ongoing. As such, addition of the probe after a PCR reaction, optionally in the presence of a recombinase, would not require the probe to comprise a polymerase blocking group. In addition, formation of a recombinase-probe-target DNA sequence complex may occur at temperatures where RPA can be performed. According to the invention, preferably, contacting the recombinase and the probe with the sample, preferably wherein the sample comprises the nucleic acid amplification product, is performed between about 20 °C and about 50 °C, preferably at less than about 45 °C, more preferably at less than about 40 °C, even more preferably at less than about 35 °C, about 30 °C, or about 25 °C. According to the invention, preferably, contacting the recombinase and the probe with the sample, preferably wherein the sample comprises the nucleic acid amplification product, is performed between 20 °C and 50 °C, preferably at less than 45 °C, more preferably at less than 40 °C, even more preferably at less than 35 °C, 30 °C, or 25 °C.

Various means for blocking different polymerases are known in the art and can be suitably selected depending on the particular assay. For example, modified nucleotides or dNTPs, such as dATP, dCTP, dGTP, dUTP, and/or dTTP can be used. Modified nucleotides or dNTPs may in theory be modified at any site of the molecule, but preferably not at a site required for incorporation of the modified nucleotide or dNTP into the DNA, at a site required for binding by a recombinase, a recombinase-loading factor, or a single-strand binding protein, or at any other site which hinders or prevents the method according to the invention. For example, a dNTP comprising a first label may not be modified in such a way that the 5′ phosphate and/or the 3′ hydroxyl of the deoxyribose of the dNTP are replaced with the first label.

The skilled person can suitably design modified dNTPs and modified nucleotides at sites for them to be used for the method of the invention. Preferably, the probe according to the invention comprises a nucleotide comprising a polymerase blocking group, preferably wherein the nucleotide is modified. Preferably, the polymerase blocking group according to the invention comprises a hydrocarbon spacer, inverted deoxynucleotide, phosphorylation, primary amine, or dideoxynucleotide, preferably wherein the hydrocarbon spacer comprises of from 3 to 12 carbon atoms, preferably of from 3 to 9, of from 3 to 6, of from 6 to 12, or of from 6 to 9 carbon atoms, more preferably wherein the hydrocarbon spacer comprises 3, 6, or 12 carbon atoms. Preferably, the polymerase blocking group or the nucleotide comprising polymerase blocking group according to the invention is at the 3′ end of the probe, wherein the 3′ end is of from 0 to 10 nucleotides of the 3′ end nucleotide of the probe including the 3′ end nucleotide. Preferably, the 3′ end is of from 0 to 7, of from 0 to 5, of from 0 to 3, of from 3 to 10, of from 5 to 10, of from 7 to 10, of from 3 to 5, or of from 3 to 7 of the 3′ end nucleotide of the probe including the 3′ end nucleotide. Preferably, the polymerase blocking group or the nucleotide comprising polymerase blocking group according to the invention respectively is at or is the 3′ end nucleotide or nucleotide 1, 2, 3, 4, 5, 6, 7, 8, 9, or 10 as counted from the 3′ end nucleotide of the probe.

Various means for labeling a nucleotide, dNTP, primer, or probe are known in the art and can be suitably selected depending on the particular assay. For example, fluorescent compounds, enzymes, high-affinity proteins, or radioactive elements can be attached by various means. Haptens have been extensively investigated as labels for nucleotides or dNTPs. The label according to the invention preferably comprises a hapten. The label according to the invention preferably comprises a covalently attached label. Preferably, the primer or probe comprising a first, second, third, or fourth label comprises a nucleotide comprising the respective first, second, third, or fourth label. Preferably, the label, preferably the second, third, or fourth label, comprises fluorescein or its derivatives, digoxigenin, Texas Red, dansyl, Cascade Blue, or dinitrophenol (DNP). Many derivatives of fluorescein are known, but preferred fluorescein derivatives comprise fluorescein amidites such as 6-FAM, or fluorescein isothiocyanate (FITC). Preferably, the label, preferably the second, third, or fourth label, comprises a fluorescent compound or a derivative thereof, a fluorogenic compound or a derivative thereof, or a steroid or a derivative thereof. Preferably, the label, preferably the second, third, or fourth label, comprises a fluorescent compound comprising aromatic groups or a fluorogenic compound comprising aromatic groups. The first label according to the invention preferably comprises biotin.

According to the invention, the second, third, or fourth label or the nucleotide comprising the second, third, or fourth label preferably is at the 5′ end of the probe, wherein the 5′ end is of from 0 to 10 nucleotides of the 5′ end nucleotide of the probe, including the end nucleotide. Preferably, the 5′ end is of from 0 to 7, of from 0 to 5, of from 0 to 3, of from 3 to 10, of from 5 to 10, of from 7 to 10, of from 3 to 5, or of from 3 to 7 of the 5′ end nucleotide of the probe including the 5′ end nucleotide. Preferably, the second, third, or fourth label or the nucleotide comprising the second, third, or fourth label according to the invention respectively is at or is the 5′ end nucleotide or nucleotide 1, 2, 3, 4, 5, 6, 7, 8, 9, or 10 as counted from the 5′ end nucleotide of the probe. According to the invention, the second, third, or fourth label or the nucleotide comprising the second, third, or fourth label preferably is at the 3′ end of the probe, wherein the 3′ end is of from 0 to 10 nucleotides of the 3′ end nucleotide of the probe, including the end nucleotide. Preferably, the 3′ end is of from 0 to 7, of from 0 to 5, of from 0 to 3, of from 3 to 10, of from 5 to 10, of from 7 to 10, of from 3 to 5, or of from 3 to 7 of the 3′ end nucleotide of the probe including the 3′ end nucleotide. Preferably, the second, third, or fourth label or the nucleotide comprising the second, third, or fourth label according to the invention respectively is at or is the 3′ end nucleotide or nucleotide 1, 2, 3, 4, 5, 6, 7, 8, 9, or 10 as counted from the 3′ end nucleotide of the probe. According to the invention, the second, third, or fourth label or the nucleotide comprising the second, third, or fourth label preferably is in the middle of the probe, wherein the middle is of from 0 to 10 nucleotides of the center nucleotide of the probe, including the center nucleotide. Preferably, the middle is of from 0 to 7, of from 0 to 5, of from 0 to 3, of from 3 to 10, of from 5 to 10, of from 7 to 10, of from 3 to 5, or of from 3 to 7 of the center nucleotide of the probe, including the center nucleotide. Preferably, the second, third, or fourth label or the nucleotide comprising the second, third, or fourth label according to the invention respectively is at or is the center nucleotide of the probe or nucleotide 1, 2, 3, 4, 5, 6, 7, 8, 9, or 10 as counted from the center nucleotide of the probe.

According to the invention, the second label preferably is a different label than the first label. Preferably, the first label according to the invention comprises or is biotin or its derivatives and the second label is not or does not comprise biotin or its derivatives. Preferably, the first label according to the invention comprises or is biotin and the second label is not or does not comprise biotin. Preferably, nucleotides or dNTP monomers comprising the first label comprise biotin-labeled dCTP, biotin-labeled dUTP, biotin-labeled deoxyguanosine triphosphate (dGTP), biotin-labeled deoxythymidine (dTTP), and/or biotin-labeled deoxyadenosine triphosphate (dATP), more preferably biotin-labeled dCTP and/or biotin-labeled dUTP.

According to the invention, the nucleotides or dNTP monomers comprising a first label are preferably biotin-labeled nucleotides or biotin-labeled dNTP monomers, preferably wherein the biotin is covalently linked to the nucleotide or dNTP, preferably via a linker of from 11 to 18 atoms. Preferably, the linker is of from 11 to 16, of from 11 to 14, of from 14 to 18, of from 14 to 16, or of from 16 to 18 atoms, more preferably the linker is 11, 14, 16, or 18 atoms, most preferably the linker is 16 atoms. Preferably, the biotin-labeled dNTP monomers comprise biotin-labeled dCTP, biotin-labeled dATP, biotin-labeled dGTP, biotin-labeled dTTP, and/or biotin-labeled dUTP, more preferably biotin-labeled dCTP and/or biotin-labeled dUTP, even more preferably biotin-16-dCTP and/or biotin-16-dUTP. Preferably, the biotin-labeled nucleotides comprise a biotin-labeled cytosine nucleotide, biotin-labeled adenine nucleotide, biotin-labeled guanine nucleotide, biotin-labeled thymidine nucleotide, and/or biotin-labeled uracil nucleotide, more preferably a biotin-labeled cytosine nucleotide and/or biotin-labeled uracil nucleotide.

According to the invention, the probe preferably comprises of from about 30 to about 70%, preferably of from about 40 to about 50%, dCTP and/or dGTP monomers based on the total number of dNTP monomers of the probe, optionally wherein the monomers comprise a first label. Preferably, the probe comprises of from about 30 to about 60%, of from about 30 to about 50%, of from about 40 to about 70%, or of from about 40 to about 60% dCTP and/or dGTP monomers based on the total number of dNTP monomers of the probe, optionally wherein the monomers comprise a first label. According to the invention, the probe preferably comprises of from about 30 to about 70%, preferably of from about 40 to about 50%, cytosine and/or guanine nucleotides based on the total number of nucleotides of the probe, optionally wherein the nucleotides comprise a first label. Preferably, the probe comprises of from about 30 to about 60%, of from about 30 to about 50%, of from about 40 to about 70%, or of from about 40 to about 60% cytosine and/or guanine nucleotides based on the total number of nucleotides of the probe, optionally wherein the nucleotides comprise a first label.

According to the invention, the probe preferably comprises of from 30 to 70%, preferably of from 40 to 50%, dCTP and/or dGTP monomers based on the total number of dNTP monomers of the probe, optionally wherein the monomers comprise a first label. Preferably, the probe comprises of from 30 to 60%, of from 30 to 50%, of from 40 to 70%, or of from 40 to 60% dCTP and/or dGTP monomers based on the total number of dNTP monomers of the probe, optionally wherein the monomers comprise a first label. According to the invention, the probe preferably comprises of from 30 to 70%, preferably of from 40 to 50%, cytosine and/or guanine nucleotides based on the total number of nucleotides of the probe, optionally wherein the nucleotides comprise a first label. Preferably, the probe comprises of from 30 to 60%, of from 30 to 50%, of from 40 to 70%, or of from 40 to 60% cytosine and/or guanine nucleotides based on the total number of nucleotides of the probe, optionally wherein the nucleotides comprise a first label.

In US20110059506A1 it has been proposed that during RPA progressively larger oligonucleotides can decrease the rate of invasion/extension, although it may be desirable to extend the length of the oligonucleotide to accommodate a duplex region in the 3′ or 5′ region of the searching oligonucleotide. Therefore, according to the invention, the probe or the primer preferably has a length of at least 25 nucleotides, more preferably of at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, or at least 60 nucleotides. Preferably, the probe or primer has a length of from 25 to 100 nucleotides, more preferably of from 26, 27, 28, 29, 30, 31, 32, 33, 34, or 35 to 50, 51, 52, 53, 54, 55, 56, 57, 58, 59, 60, 65, 70, 80, 90, or 100 nucleotides, even more preferably a length of from 30, 35, or 40 to 45, 50, 55, or 60 nucleotides. Preferably, the probe or primer has a length of from 25 to 100 nucleotides, more preferably of from 26 to 100, of from 26 to 95, of from 26 to 90, of from 26 to 85, of from 26 to 80, of from 26 to 75, of from 26 to 70, of from 26 to 65, of from 26 to 60, of from 27 to 100, of from 27 to 95, of from 27 to 90, of from 27 to 85, of from 27 to 80, of from 27 to 75, of from 27 to 70, of from 27 to 65, of from 28 to 100, of from 28 to 95, of from 28 to 90, of from 28 to 85, of from 28 to 80, of from 28 to 75, of from 28 to 70, of from 28 to 65, of from 30 to 100, of from 30 to 95, of from 30 to 90, of from 30 to 85, of from 30 to 80, of from 30 to 75, of from 30 to 70, of from 30 to 65, of from 32 to 95, of from 32 to 90, of from 32 to 85, of from 32 to 80, of from 32 to 75, of from 32 to 70, of from 32 to 65, of from 32 to 60, of from 35 to 100, of from 35 to 95, of from 35 to 90, of from 35 to 85, of from 35 to 80, of from 35 to 75, of from 35 to 70, of from 35 to 65, of from 38 to 100, of from 38 to 95, of from 38 to 90, of from 38 to 85, of from 38 to 80, of from 38 to 75, of from 38 to 70, of from 38 to 65, of from 40 to 100, of from 40 to 95, of from 40 to 90, of from 40 to 85, of from 40 to 80, of from 40 to 75, of from 40 to 70, or of from 40 to 65 nucleotides.

The DNA-dependent DNA polymerase according to the invention preferably is a strand-displacing DNA-dependent DNA polymerase, thereby enabling an RPA reaction.

Recombinases play a central role in homologous recombination in a variety of organisms. Such recombinases have been described in archaea, bacteria, eukaryotes, and viruses. The recombinase according to the invention preferably is a recombinase that facilitates homologous recombination. Preferably, the recombinase according to the invention is a recombinase capable of targeting an oligonucleotide, such as a primer or an oligonucleotide probe, to a target DNA sequence, preferably in double-stranded DNA. Preferably, the recombinase is capable of pairing an oligonucleotide, such as a primer or an oligonucleotide probe, with a complementary DNA sequence, preferably in double-stranded DNA. Preferably, the recombinase according to the invention is a recombinase selected from the group consisting of a RecA, DrRecA, Rad51, RadA, or UvsX recombinase, or a homologue thereof. The RecA family of recombinases is encoded by several organisms. Preferably, the recombinase is RecA of Escherichia coli or Deinococcus radiodurans. Preferably, the recombinase is UvsX recombinase of bacteriophage T4. Preferably, the recombinase is Rad51 and its cofactors. Preferably, the recombinase is DrRecA of Deinococcus radiodurans. These recombinases facilitate the formation of a D-loop. Preferably, the recombinase does not use or need a nucleoside triphosphate, for example ATP or GTP, for formation or maintenance of a D-loop. Preferably, the recombinase does not use or need an analogue of a nucleoside triphosphate, for example ATPγS, for formation or maintenance of a D-loop.

The method according to the invention preferably is a one-pot reaction. A one-pot reaction simplifies the method and reduces (cross-)contamination of samples. Preferably, the one-pot reaction comprises performing all steps of the method simultaneously. Preferably, the one-pot reaction comprises performing the method in one container. Preferably, the one-pot reaction comprises no purification or isolation of compounds.

According to the invention, the method preferably comprises contacting the sample with a means for modulating binding of the recombinase or the labeled probe to the nucleic acid amplification product, preferably the sample comprising the nucleic acid amplification product or the isolated nucleic acid amplification product. As disclosed above, binding of the labeled probe or the recombinase-labeled probe complex to the target DNA sequence of the nucleic acid amplification product facilitates subsequent detection of the nucleic acid amplification product. Modulating this binding in order to maintain or increase the binding is hence preferred. Preferably, the means for modulating binding of the recombinase or the labeled probe to the nucleic acid amplification product comprises an ATP analogue, RecO, RecT, RecF, UvsY, a double-stranded DNA destabilizing agent, a triple-stranded DNA stabilizing agent, a single-stranded DNA-binding protein, or any combination thereof.

It has been speculated that recombinase binding to single-stranded DNA can be stabilized by the use of non-hydrolyzable or hydrolysis-resistant ATP analogues, since in the presence of the non-hydrolyzable ATP analogue, adenosine 5′-gamma-thiotriphosphate (ATP-γ-S), hydrolysis of ATP-γ-S by the recombinase and subsequent disassembly of the binding of the recombinase to single-stranded DNA is inhibited (see US20110059506A1). It is proposed that the stability of the RecA/ATP-γ-S/DNA complex may be helpful in targeting the recombinase, but may also be detrimental and unpractical for DNA amplification.

According to the invention, the method preferably further comprises adding an ATP analogue before, during, or after amplifying the target DNA sequence, preferably after amplifying. Preferably, amplifying the target DNA sequence comprises performing an RPA reaction and adding the ATP analogue during or after the RPA reaction. Adding the ATP analogue before or during amplifying the target DNA sequence preferably comprises contacting the sample with the ATP analogue. Adding the ATP analogue after amplifying the target DNA sequence preferably comprises contacting the nucleic acid amplification product with the ATP analogue, optionally after isolating the nucleic acid amplification product. The term ‘adding’ herein is understood to mean contacting the sample with.

Preferably, the ATP analogue according to the invention is a non-hydrolyzable or hydrolysis-resistant ATP analogue. Preferably, the ATP analogue is selected from the group consisting of AMPPNP adenylyl imidodiphosphate (AMPPNP), adenylyl methylenediphosphate (AMPPCP), alpha, beta-methylene-triphosphonate (AMPCPP), alpha-thio-5-triphosphate (ATPαS), and adenosine 5′-gamma-thiotriphosphate (ATP-γ-S). Preferably, the ATP analogue is ATP-γ-S.

It was found that adding an ATP analogue together with ATP is advantageous for the method of the invention. According to the invention, the method preferably further comprises adding an ATP analogue and ATP before, during, or after amplifying the target DNA sequence, preferably after amplifying. Preferably, amplifying the target DNA sequence comprises performing an RPA reaction and adding the ATP analogue and ATP during or after the RPA reaction. Adding the ATP analogue and ATP before or during amplifying the target DNA sequence preferably comprises contacting the sample with the ATP analogue and ATP. Adding the ATP analogue and ATP after amplifying the target DNA sequence preferably comprises contacting the nucleic acid amplification product with the ATP analogue and ATP, optionally after isolating the nucleic acid amplification product. Preferably, adding the ATP analogue and ATP comprises contacting the sample with the ATP analogue and ATP in a molar ratio of from 0.5 to 2:5 to 15, preferably of about 1:10. Preferably, adding the ATP analogue and ATP comprises contacting the sample with the ATP analogue and ATP in a molar ratio of from 0.5 to 1.5:5 to 15, of from 1 to 2:5 to 15, of from 0.5 to 1.5:7 to 13, or of from 1 to 2:7 to 13. Preferably, adding the ATP analogue and ATP comprises contacting the sample with the ATP analogue and a molar amount of ATP that is at least about 2-fold the molar amount of the ATP analogue, preferably at least about 3, about 4, about 5, about 6, about 7, about 8, about 9, or about 10-fold the molar amount of the ATP analogue. Preferably, adding the ATP analogue and ATP comprises contacting the sample with the ATP analogue and a molar amount of ATP that is at least 2-fold the molar amount of the ATP analogue, preferably at least 3, 4, 5, 6, 7, 8, 9, or 10-fold the molar amount of the ATP analogue.

Alternatively, or additionally, ATP may be added by using an ATP regeneration system, for example by using creatine phosphate and creatine kinase, wherein creatine kinase phosphotylates adenosine monophosphate (AMP) or adenosine diphosphate (ADP) into ATP in the presence of creatine phosphate, or as disclosed in EP3249056B1. An ATP regeneration system commonly comprises using an enzymatic reaction that continuously produces ATP from ADP or AMP, preferably ADP, and a secondary phosphate donor. According to the invention, the method preferably further comprises adding an ATP analogue and an ATP regeneration system before, during, or after amplifying the target DNA sequence, preferably after amplifying. Preferably, amplifying the target DNA sequence comprises performing an RPA reaction and adding the ATP analogue and the ATP regeneration system during or after the RPA reaction. Adding the ATP analogue and the ATP regeneration system before or during amplifying the target DNA sequence preferably comprises contacting the sample with the ATP analogue and the ATP regeneration system. Adding the ATP analogue and the ATP regeneration system after amplifying the target DNA sequence preferably comprises contacting the nucleic acid amplification product with the ATP analogue and the ATP regeneration system, optionally after isolating the nucleic acid amplification product. Preferably, amplifying the target DNA sequence comprises performing an RPA reaction. Preferably, the ATP regeneration system comprises creatine phosphate and creatine kinase, acetate kinase and acetyl phosphate, pyruvate kinase and phosphoenolpyruvate and/or phospho-phenyl-pyruvate, or polyphosphate kinase and polyphosphate, more preferably creatine phosphate and creatine kinase.

In US20110059506A1 it is proposed that RecA-single-stranded DNA complexes can be stabilized by the RecO, RecT, and/or RecF proteins and that UvsX-single-stranded DNA complexes can be stabilized by the T4 UvsY protein. Hence, according to the invention, the sample is preferably further contacted with RecO, RecT, RecF, or UvsY. Preferably, the sample is contacted with UvsY when the sample is contacted with UvsX recombinase. Preferably, the sample is contacted with RecO, RecT, or RecF when the sample is contacted with RecA recombinase.

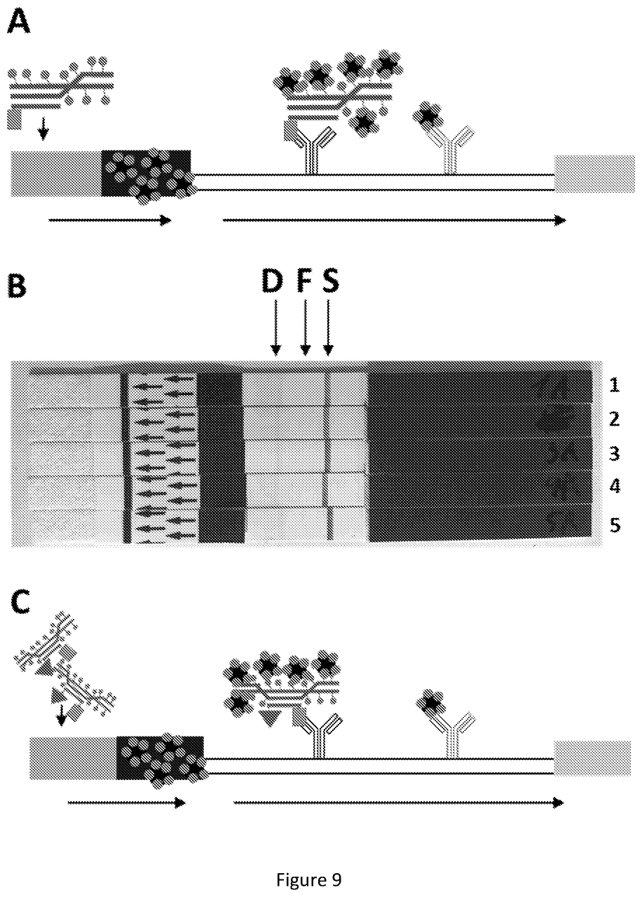

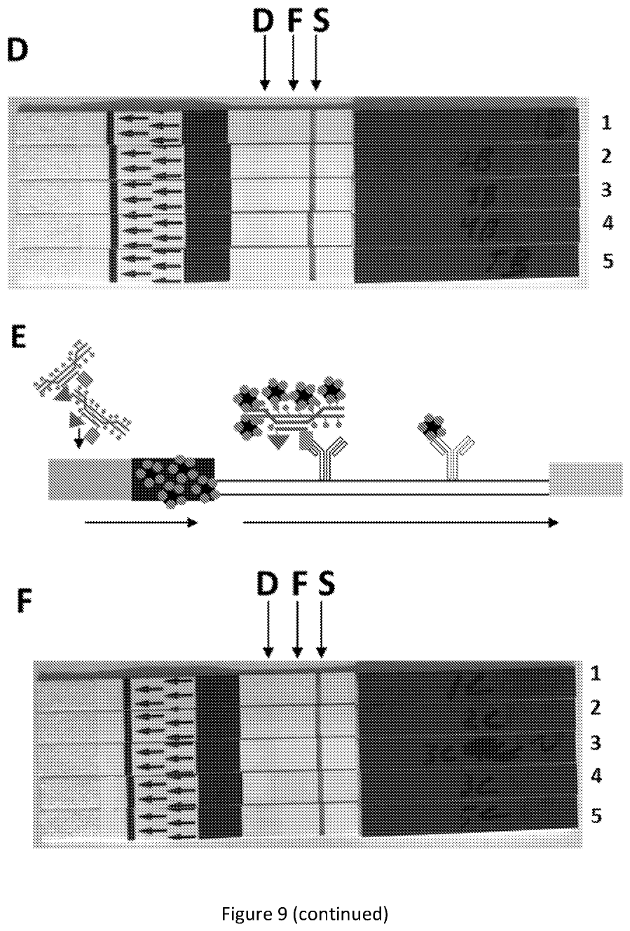

According to the invention, the method preferably further comprises contacting the sample, preferably the sample comprising the nucleic acid amplification product or the isolated nucleic acid amplification product by for example DNA purification, with a double-stranded DNA destabilizing agent and/or a triple-stranded DNA stabilizing agent. Preferably, the triple-stranded DNA stabilizing agent is an agent capable of stabilizing a complex comprising a recombinase/single-stranded DNA complex and double stranded DNA. Preferably, the triple-stranded DNA stabilizing agent is an agent capable of stabilizing a D-loop, preferably a D-loop generated by a recombinase. Some double-stranded double-stranded DNA destabilizing agents may (indirectly) act as triple-stranded DNA stabilizing agents and vice versa. Agents that destabilize double-stranded DNA may be used to facilitate the binding of the probe according to the invention to the target DNA sequence, thereby indirectly increasing the stability of D-loop. Examples of double-stranded DNA destabilizing agents include, but are not limited to, polyethylene glycol (PEG), trehalose, ionic liquid, betaine, proline, adenovirus DNA-Binding protein, calf thymus helix-destabilizing protein, and ribonuclease (Ozay and McCalla, Sensors and Actuators Reports, Volume 3, November 2021, 100033). Ribonuclease can destabilize RNA-DNA duplexes. Preferably, PEG has an average molecular weight of 1450, 6000, or of from 15000 to 20000. In the present application, the use of ionic liquid surprisingly enabled improved detection of a nucleic acid amplification product according to the invention, see FIG. 9F. This seemed to be in particular when a probe was used that comprised a DNA sequence complementary to a middle region of the target DNA sequence, when a paranemic joint was formed. An example of a triple-stranded DNA stabilizing agent is potassium, preferably KCl (Teng et al., Molecules, 2020, 25(2), 387). A triple-stranded DNA stabilizing agent comprising potassium may further comprise PEG, preferably PEG 200. Further double-stranded DNA destabilizing agents and triple-stranded DNA stabilizing agents are known in the art and can be suitably selected for use according to the invention.

According to the invention, the sample is preferably further contacted with a single-stranded DNA-binding protein. Preferably, the single stranded DNA-binding protein comprises gp32 of the bacteriophage T4 or single-stranded binding protein SSB of Escherichia coli. Preferably, the sample is contacted with gp32 when the sample is contacted with UvsX recombinase. Preferably, the sample is contacted with SSB when the sample is contacted with RecA recombinase. The single-stranded DNA-binding protein helps to stabilize the displaced DNA strand, which facilitates the formation of a D-loop.

The presence or absence of a target ribonucleic acid (RNA) sequence may also be determined by the present invention. To achieve this, the target RNA sequence is first to be converted to DNA, for example to complementary DNA (cDNA) via a process termed reverse transcription, which is a well-known process in the art. Hence, a target DNA sequence is generated from a target RNA sequence. According to the invention, the method may further comprise contacting the sample with an RNA-dependent DNA polymerase prior to or during step a or b to produce the target DNA sequence.

According to the invention, the nucleic acid sequence complementary to the target DNA sequence of the probe or the nucleic acid sequence of the target DNA sequence to which the probe hybridizes preferably does not overlap with the nucleic acid sequence to which the first primer or the second primer hybridizes. According to the invention, the nucleic acid sequence complementary to the target DNA sequence of the probe or the nucleic acid sequence of the target DNA sequence to which the probe hybridizes preferably partially overlaps with the nucleic acid sequence to which the first primer or the second primer hybridizes, preferably wherein the partial overlap is of from 1 to 10, of from 1 to 7, of from 1 to 5, of from 1 to 3, of from 3 to 7, of from 3 to 5, or of from 5 to 7 nucleotides. The extent of the partial overlap of the primer(s) with the probe sequence depends on the length of the probe and the requirement for a potential mismatch discrimination and can be suitably selected by a person skilled in the art.

According to the invention, contacting the sample preferably comprises contacting the sample with a smaller or equal amount of probe compared to the amount of the first primer and/or the second primer based on the number of moles, preferably wherein the molar ratio of each of the first primer and the second primer to the probe is of from 1:1 to 100:1, of from 1:1 to 75:1, of from 1:1 to 25:1, of from 10:1 to 100:1, of from 25:1 to 100:1, of from 10:1 to 75:1, of from 10:1 to 25:1, or of from 25:1 to 75:1. Preferably, contacting the sample comprises contacting the sample with primers and a probe in a molar ratio of primers to probe of from 1:1 to 100:1, of from 1:1 to 75:1, of from 1:1 to 25:1, of from 10:1 to 100:1, of from 25:1 to 100:1, of from 10:1 to 75:1, of from 10:1 to 25:1, or of from 25:1 to 75:1.

The first binding agent according to the invention preferably comprises a binding agent capable of binding biotin, preferably avidin, streptavidin, neutravidin, an anti-biotin antibody, or a binding agent specific for biotin. The second binding agent according to the invention preferably comprises an antibody. According to the invention, the first binding agent preferably comprises a binding agent capable of binding biotin, preferably avidin, streptavidin, neutravidin, an anti-biotin antibody, or a binding agent specific for biotin and the second binding agent comprises a binding agent or antibody that is not capable of binding biotin or does not specifically bind biotin.

Various means of detection of labeled compounds are known in the art. Detection according to the invention is preferably achieved via a visual readout, preferably without the aid of advanced equipment. According to the invention, detecting the nucleic acid amplification product preferably comprises using a lateral flow test, lateral flow device, enzyme-linked immunosorbent assay (ELISA), magnetic immunoassay, or flocculation assay. According to the invention, the first binding agent preferably comprises a nanoparticle, colored (nano)particle, gold (nano)particle, latex, fluorescent compound, luminescent compound, or a magnetic compound. The increased local concentration of first label after amplification may facilitate (visual) detection by the above first binding agents. Real-time detection during amplification is also possible using the method of the invention. Various methods for achieving real-time detection during nucleic acid amplification can be used by combining known methods for real-time detection of real-time PCR or real-time RPA. For example, non-specific fluorescent compounds that intercalate with any double-stranded DNA may be contacted with the sample. Alternatively, the probe according to the invention may comprise a fluorescent label, which can be detected only after binding of the probe to its complementary sequence. For example, the probe may be designed to be compatible with the TwistAmp® exo kit, wherein an exonuclease cuts a specific residue in the hybridized probe which disrupts the quenching effect of an attached quenching compound on an attached fluorophore. According to the invention, detecting the nucleic acid amplification product preferably is performed during amplification of the target DNA sequence, preferably wherein the probe, the first label, or the second label, comprises a fluorescent compound, more preferably the probe.

The present invention can also be used for multiplexing assays, wherein simultaneously multiple target DNA sequences can be amplified and detected. According to the first method of the invention, the method preferably further comprises contacting the sample with: a third primer and a fourth primer for amplifying a second target DNA sequence; and a second oligonucleotide probe comprising a nucleic acid sequence complementary to a second target DNA sequence and a third label. According to the first method of the invention, detecting the nucleic acid amplification product in step c preferably further comprises binding the third label of the second probe bound to the nucleic acid amplification product to a third binding agent, preferably wherein the first label and the third label do not bind to the second binding agent, the second label and the third label do not bind to the first binding agent, and the first label and the second label do not bind to the third binding agent. Preferably, the first binding agent is capable of binding the first label, the second binding agent is capable of binding the second label, and the third binding agent is capable of binding the third label. Preferably, the first binding agent is specific for the first label, the second binding agent is specific for the second label, and the third binding agent is specific for the third label. Similarly, additional different primer sets and oligonucleotide probes having different labels can be used to detect additional different target DNA sequences, which can be detected by additional binding agents capable of binding or preferably specific for the respective different labels. For example, a fifth and sixth primer for amplifying a third target DNA sequence, and a third oligonucleotide probe comprising a nucleic acid sequence complementary to a third target DNA sequence and a fourth label; a seventh and eighth primer for amplifying a fourth target DNA sequence, and a fourth oligonucleotide probe comprising a nucleic acid sequence complementary to a fourth target DNA sequence and a fifth label, etc.

According to the second method of the invention, the method preferably further comprises contacting the sample with: a third primer and a fourth primer for amplifying a second target DNA sequence, wherein the fourth primer comprises a third label and hybridizes to a first strand of DNA of the second target DNA sequence, and a second oligonucleotide probe comprising a nucleic acid sequence complementary to the second target DNA sequence and one or more nucleotides comprising a first label, preferably wherein the probe hybridizes to the first strand or the second strand of DNA of the second target DNA sequence, the second strand being complementary to the first strand. According to the second method of the invention, detecting the nucleic acid amplification product in step c preferably further comprises binding the third label of the fourth primer bound to the nucleic acid amplification product to a third binding agent, preferably wherein the first label and the third label do not bind to the second binding agent, the second label and the third label do not bind to the first binding agent, and the first label and the second label do not bind to the third binding agent. Preferably, the first binding agent is capable of binding the first label, the second binding agent is capable of binding the second label, and the third binding agent is capable of binding the third label. Preferably, the first binding agent is specific for the first label, the second binding agent is specific for the second label, and the third binding agent is specific for the third label. Similarly, additional different primer sets, wherein one of the primers has a different label, and oligonucleotide probes targeting different DNA sequences can be used to detect additional different target DNA sequences, which can be detected by additional binding agents capable of binding or preferably specific for the respective different labels. For example, a fifth and sixth primer for amplifying a third target DNA sequence, wherein the sixth primer comprises a third label and hybridizes to a first strand of DNA of the third target DNA sequence, and a third oligonucleotide probe comprising a nucleic acid sequence complementary to the third target DNA sequence and one or more nucleotides comprising a first label, wherein the probe preferably hybridizes to the first strand or the second strand of DNA of the third target DNA sequence, the second strand being complementary to the first strand; a seventh and eighth primer for amplifying a fourth target DNA sequence, wherein the eighth primer comprises a fourth label and hybridizes to a first strand of DNA of the second target DNA sequence, and a fourth oligonucleotide probe comprising a nucleic acid sequence complementary to the fourth target DNA sequence and one or more nucleotides comprising a first label, wherein the probe preferably hybridizes to the first strand or the second strand of DNA of the fourth target DNA sequence, the second strand being complementary to the first strand, etc.

Detection of a multiplexed method of the invention can be done by various means known in the art, for example by capturing each of the second and further labels by respective (immobilized) binding agents separated by time or location, after which each of the target DNA sequences is isolated and can be detected separately via the first binding agent.

A preferred method of detecting the nucleic acid amplification product according to the invention comprises the use of a lateral flow device, also known as a lateral flow test (Sajid et al., Journal of Saudi Chemical Society. 2015 November; Vol 19, Issue 6, p 689-705). Lateral flow devices are simple and affordable point-of-care testing devices to detect the presence of a target substance in a (liquid) sample without the need for specialized and costly equipment. A typical lateral flow device comprises a strip comprising a sample application zone, a reagent zone, a test zone, and an end zone. The liquid sample to be analyzed is applied on the sample application zone and passes through the different zones of the lateral flow strip towards the end zone. The reagent zone typically comprises reagents that enable the subsequent detection of the target substance in the test zone. The test zone typically comprises immobilized binding agents that interact with the target substance, thereby indicating the presence of the target substance, usually as a visual line.

According to the invention, detecting the nucleic acid amplification product preferably comprises:
a. providing a lateral flow device comprising a first binding agent capable of binding the first label and a second binding agent capable of binding the second label, preferably wherein the second binding agent is immobilized, preferably immobilized on the lateral flow device;
b. applying the nucleic acid amplification product to a lateral flow test strip of the lateral flow device; and
c. detecting the product, if present, on a test zone of the lateral flow strip.

Preferably, before step b of applying the nucleic acid amplification product to a lateral flow test strip of the lateral flow device, a step of diluting the nucleic acid amplification product is performed, preferably using a buffer with a pH of from about 6, about 6.1, about 6.2, about 6.3, about 6.4, about 6.5, about 6.6, about 6.7, about 6.8, or about 6.9 to about 7.1, about 7.2, about 7.3, about 7.4, about 7.5, about 7.6, about 7.7, about 7.8, about 7.9, or about 8, preferably of about 7; and is followed by step b of applying the diluted nucleic acid amplification product to a lateral flow test strip of the lateral flow device. Preferably, before step b of applying the nucleic acid amplification product to a lateral flow test strip of the lateral flow device, a step of diluting the nucleic acid amplification product is performed, preferably using a buffer with a pH of from about 6 to about 8, of from about 6 to about 7.7, from about 6 to about 7.5, from about 6 to about 7.3, from about 6 to about 7.1, from about 6.3 to about 8, from about 6.3 to about 7.7, from about 6.3 to about 7.5, from about 6.3 to about 7.3, from about 6.3 to about 7.1, from about 6.5 to about 8, from about 6.5 to about 7.7, from about 6.5 to about 7.5, from about 6.5 to about 7.3, from about 6.5 to about 7.1, from about 6.7 to about 8, from about 6.7 to about 7.7, from about 6.7 to about 7.5, from about 6.7 to about 7.3, from about 6.7 to about 7.1, from about 6.9 to about 8, from about 6.9 to about 7.7, from about 6.9 to about 7.5, from about 6.9 to about 7.3, from about 6.9 to about 7.1, or of about 7; and is followed by step b of applying the diluted nucleic acid amplification product to a lateral flow test strip of the lateral flow device.

Preferably, before step b of applying the nucleic acid amplification product to a lateral flow test strip of the lateral flow device, a step of diluting the nucleic acid amplification product is performed, preferably using a buffer with a pH of from 6, 6.1, 6.2, 6.3, 6.4, 6.5, 6.6, 6.7, 6.8, or 6.9 to 7.1, 7.2, 7.3, 7.4, 7.5, 7.6, 7.7, 7.8, 7.9, or 8, preferably of about 7; and is followed by step b of applying the diluted nucleic acid amplification product to a lateral flow test strip of the lateral flow device. Preferably, before step b of applying the nucleic acid amplification product to a lateral flow test strip of the lateral flow device, a step of diluting the nucleic acid amplification product is performed, preferably using a buffer with a pH of from 6 to 8, of from 6 to 7.7, from 6 to 7.5, from 6 to 7.3, from 6 to 7.1, from 6.3 to 8, from 6.3 to 7.7, from 6.3 to 7.5, from 6.3 to 7.3, from 6.3 to 7.1, from 6.5 to 8, from 6.5 to 7.7, from 6.5 to 7.5, from 6.5 to 7.3, from 6.5 to 7.1, from 6.7 to 8, from 6.7 to 7.7, from 6.7 to 7.5, from 6.7 to 7.3, from 6.7 to 7.1, from 6.9 to 8, from 6.9 to 7.7, from 6.9 to 7.5, from 6.9 to 7.3, from 6.9 to 7.1, or of about 7; and is followed by step b of applying the diluted nucleic acid amplification product to a lateral flow test strip of the lateral flow device.

Preferably, the buffer comprises a non-ionic detergent, preferably a non-ionic detergent that does not substantially denaturize or aggregate proteins. Preferably, the buffer comprises a non-denaturing non-ionic detergent. Various non-denaturing non-ionic detergents are known in the art and can be suitably selected, examples are Tween-20, Tween-80, digitonin, n-dodecyl-β-D-maltoside, Triton X-100, Triton X-114, Nonidet P-40 (NP-40), Igepal® CA-630. Alternatively, the buffer preferably comprises a non-denaturing ionic detergent such as cholic acid.

It was found that diluting the nucleic acid amplification product improves the detection of the product. An overabundance of the nucleic acid amplification product and/or crowding agents in the RPA reaction may interfere with producing a detectable signal, for example by decreasing the rate wicking of the sample pad (i.e. sample application zone) and/or of the capillary flow in a lateral flow assay, by reducing the amount of binding agent per amplified target DNA sequence, and/or by binding the first binding agent without binding the immobilized second binding agent, thereby resulting in a low signal.

Another preferred method of detecting the nucleic acid amplification product according to the invention comprises flocculation. Detection of amplified nucleic acids by visual inspection after flocculation is disclosed in US2017029881A1. Herein, a complex is formed by flocculation of the nucleic acid and a particle capable of binding the nucleic acid. US2017029881A1 defines flocculation as a reversible non-covalent aggregation of colloids, which subsequently enables visual inspection of the aggregate. The present invention enables visual detection of the nucleic acid amplification product via flocculation because of the high concentration of the first label that is present in the amplification product, which can subsequently cause a high local concentration of the first binding agent that binds the first label resulting in a visual signal.

According to the invention, detecting the nucleic acid amplification product preferably comprises combining the nucleic acid amplification product and the first binding agent, wherein the product and the first binding agent are capable of forming a complex which can be detected visually, wherein the complex is formed by flocculation, preferably by flocculation of the first binding agent, more preferably of flocculation of the first binding agent by the nucleic acid amplification product. Preferably, the method further comprises that the presence or relative amount of nucleic acid amplification product is determined by the presence of, or increased, flocculation, compared to the flocculation observed in the absence of, or at a lower amount or concentration of, the nucleic acid amplification product. Preferably, the first binding agent comprises avidin, streptavidin, neutravidin, an anti-biotin antibody, or a binding agent specific for biotin bound to one or more colored, silver, or gold nanoparticles, preferably gold nanoparticles. Preferably, the nanoparticles have a diameter of from 4 or 5 to 50 or 55 nm. Preferably, the nanoparticles have a silica shell with a diameter of from 2 to 4 nm. Preferably, the nanoparticles comprise polyethylene glycol polymers.

Preferably, the method further comprises diluting the complex using a buffer with a pH of from about 6, about 6.1, about 6.2, about 6.3, about 6.4, about 6.5, about 6.6, about 6.7, about 6.8, or about 6.9 to about 7.1, about 7.2, about 7.3, about 7.4, about 7.5, about 7.6, about 7.7, about 7.8, about 7.9, or about 8, preferably of about 7. Preferably, the method further comprises diluting the complex using a buffer with a pH of from about 6 to about 8, of from about 6 to about 7.7, from about 6 to about 7.5, from about 6 to about 7.3, from about 6 to about 7.1, from about 6.3 to about 8, from about 6.3 to about 7.7, from about 6.3 to about 7.5, from about 6.3 to about 7.3, from about 6.3 to about 7.1, from about 6.5 to about 8, from about 6.5 to about 7.7, from about 6.5 to about 7.5, from about 6.5 to about 7.3, from about 6.5 to about 7.1, from about 6.7 to about 8, from about 6.7 to about 7.7, from about 6.7 to about 7.5, from about 6.7 to about 7.3, from about 6.7 to about 7.1, from about 6.9 to about 8, from about 6.9 to about 7.7, from about 6.9 to about 7.5, from about 6.9 to about 7.3, from about 6.9 to about 7.1, or of about 7.

Preferably, the method further comprises diluting the complex using a buffer with a pH of from 6, 6.1, 6.2, 6.3, 6.4, 6.5, 6.6, 6.7, 6.8, or 6.9 to 7.1, 7.2, 7.3, 7.4, 7.5, 7.6, 7.7, 7.8, 7.9, or 8, preferably of about 7. Preferably, the method further comprises diluting the complex using a buffer with a pH of from 6 to 8, of from 6 to 7.7, from 6 to 7.5, from 6 to 7.3, from 6 to 7.1, from 6.3 to 8, from 6.3 to 7.7, from 6.3 to 7.5, from 6.3 to 7.3, from 6.3 to 7.1, from 6.5 to 8, from 6.5 to 7.7, from 6.5 to 7.5, from 6.5 to 7.3, from 6.5 to 7.1, from 6.7 to 8, from 6.7 to 7.7, from 6.7 to 7.5, from 6.7 to 7.3, from 6.7 to 7.1, from 6.9 to 8, from 6.9 to 7.7, from 6.9 to 7.5, from 6.9 to 7.3, from 6.9 to 7.1, or of about 7.

Preferably, the buffer comprises bovine serum albumin, preferably of from 0.1 to 1.0% weight by volume bovine serum albumin. Preferably, the buffer comprises a non-ionic detergent, preferably a non-ionic detergent that does not substantially denaturize or aggregate proteins. Preferably, the buffer comprises a non-denaturing non-ionic detergent. Various non-denaturing non-ionic detergents are known in the art and can be suitably selected, examples are Tween-20, Tween-80, digitonin, n-dodecyl-β-D-maltoside, Triton X-100, Triton X-114, Nonidet P-40 (NP-40), Igepal® CA-630. Alternatively, the buffer preferably comprises a non-denaturing ionic detergent such as cholic acid.

The present invention further relates to an ex vivo method of diagnosing a disease or disorder in a subject, comprising determining the presence or absence of a target DNA sequence associated with the disease or disorder according to the invention in a sample which has been obtained from the subject, wherein the presence indicates a diagnosis of the disease or disorder in the subject.

The present invention relates to an ex vivo method of diagnosing a disease or disorder in a subject, comprising:
a. determining the presence or absence of a target DNA sequence associated with the disease or disorder according to the invention in a sample which has been obtained from the subject;
b. comparing the presence or absence as determined in step a to the presence or absence of the target DNA sequence associated with the disease or disorder as determined according to the invention in a sample of an individual not suffering from the disease or disorder, in a negative control, or in a positive control;
c. detecting a difference in the presence of the target DNA sequence of the subject as compared in step b; and
d. determining the extent of the difference, wherein if a sample of an individual not suffering from the disease or disorder or a negative control is compared, a difference greater than a pre-determined threshold indicates a diagnosis of the disease or disorder in the subject, wherein if a positive control is compared, no difference, a difference greater than a pre-determined threshold, or a difference less than a pre-determined threshold indicates a diagnosis of the disease or disorder in the subject.
   A pre-determined threshold can be suitably set by a skilled person and may depend for example on the background signal of the specific amplification and/or detection methods used or on the amount of the target DNA sequence associated with a diagnosis of the disease or disorder. The subject or individual may be a human or animal.

According to the invention, preferably, the control is a control sample. The control sample may or may not comprise the target DNA sequence associated with the disease or disorder, thereby being respectively a positive control sample or a negative control sample. Preferably, the control sample is a sample which has not been obtained from a subject or an individual. Preferably, the control comprises reference data. Preferably, the reference data comprises data obtained from samples comprising the target DNA sequence associated with the disease or disorder, preferably wherein the samples are obtained from subjects suffering from the disease or disorder.

According to the invention, preferably, the disease or disorder comprises cancer, autoimmune disease or disorder, hereditary disease or disorder, pain, respiratory disease or disorder, cardiovascular disease or disorder, infectious disease or disorder, thyroid disease or disorder, skin disease or disorder, diabetes, obesity, kidney disease or disorder, gastrointestinal disease or disorder, neurodegenerative disease or disorder, musculoskeletal disease or disorder, metabolic disease or disorder, or mental illness, more preferably cancer, autoimmune disease or disorder, or infectious disease or disorder.

Preferably, the target DNA sequence associated with a disease or disorder or the onset thereof comprises a DNA sequence expressing tumour antigens, tumour markers, or tumour suppressors. Preferably, the target DNA sequence associated with a disease or disorder or the onset thereof comprises a DNA sequence having a nucleotide sequence polymorphism. Preferably, the nucleotide sequence polymorphism comprises one or more somatic mutations and/or single nucleotide polymorphisms. Preferably, the target DNA sequence associated with a disease or disorder or the onset thereof comprises a DNA sequence associated with an epigenetic event, preferably DNA methylation and/or DNA hydroxymethylation.

According to the invention, preferably, the disease or disorder comprises an infectious disease or disorder, more preferably a sexually transmitted infection. Preferably, the disease or disorder comprises chlamydia, gonorrhoea, syphilis, genital herpes, acquired immunodeficiency syndrome, papilloma, or trichomoniasis. Preferably, the target DNA sequence associated with the disease or disorder comprises a sequence of the bacteria Chlamydia trachomatis, Neisseria gonorrhoeae, Treponema pallidum, the viruses human alphaherpesvirus 1, human alphaherpesvirus 2, human immunodeficiency virus 1, human immunodeficiency virus 2, human papillomavirus, or the parasite Trichomonas vaginalis.

The subject may be a human or an animal. A sample which has been obtained from the subject may comprise tissues and/or biological fluids. Such samples can be obtained in vitro, ex vivo, or in vivo. As a non-limiting example, the sample which has been obtained from the subject may be selected from tissues, organs, cells, or any isolated fraction of a human or animal subject. The sample which has been obtained from the subject may also be selected from blood, plasma, lymph, saliva, urine, stool, tears, sweat, sperm, vaginal fluids, or cerebrospinal fluid, synovial fluid, pleural fluid, peritoneal fluid, or pericardial fluid, and any fraction or extracts thereof. The sample can be obtained by any technique known in the art.

The sample which has been obtained from the subject may be subjected to a processing step prior to step a of the method of diagnosing according to the invention. This processing may facilitate determining the presence or absence of a target DNA sequence associated with the disease or disorder. Various methods of processing are known in the art. For example, thermal, mechanical, or chemical treatment of samples obtained from a subject may lyse cells enabling the liberation of DNA from the cells. Some nucleic acid amplification methods, such as RPA, may subsequently be conducted directly in certain biological samples (milk, urine, feces, pleural fluid) following thermal, mechanical, or chemical lysis.

The present invention also relates to a method of detecting a nucleic acid amplification product comprising a target DNA sequence, the method comprising:
a. contacting the nucleic acid amplification product with a recombinase and an oligonucleotide probe comprising a nucleic acid sequence complementary to the target DNA sequence and a label, thereby forming in the nucleic acid amplification product a D-loop comprising triple-stranded DNA, the probe, and the recombinase; and
b. detecting the nucleic acid amplification product, comprising detecting the probe.

As discussed above, the inventors have surprisingly found that they can detect a nucleic acid amplification product comprising a target DNA sequence and a D-loop comprising a recombinase and an oligonucleotide probe comprising a nucleic acid sequence complementary to the target DNA sequence and a label. Triple-stranded DNA in the D-loop is formed by the recombinase and the probe, which subsequently can be used for detection of the nucleic acid amplification product via detection of the label of the probe.

It is to be understood that any of the preferred features or steps according to the methods as disclosed in the present application are also preferably applicable to the above method of detecting a nucleic acid amplification product comprising a target DNA sequence where possible. For example, the probe may preferably comprise a first label or a second label according to the invention, the probe may preferably comprise a polymerase blocking group according to the invention, the recombinase may preferably be a recombinase selected from the group consisting of a RecA, DrRecA, Rad51, RadA, or UvsX recombinase, or a homologue thereof, the method may preferably further comprise adding an ATP analogue, more preferably a non-hydrolyzable or hydrolysis-resistant ATP analogue, etc.

Preferably, according to the invention, detecting the nucleic acid amplification product comprises binding the label of the probe bound to the nucleic acid amplification product to a binding agent. Preferably, the binding agent is attached to a solid support. Preferably, detecting the nucleic acid amplification product comprises using a lateral flow test, lateral flow device, enzyme-linked immunosorbent assay, magnetic immunoassay, or flocculation assay.

According to any method of the invention, preferably the method is nuclease-free or protease-free. Preferably, the method is nuclease-free and protease-free.

The present invention provides devices and systems to amplify a target DNA sequence and/or to detect the nucleic acid amplification product of the present invention. The devices and systems may include further features that enable or facilitate their intended use in combination with features that are disclosed by the methods of the present invention. The devices and systems according to the invention are suitable for use in the methods according to the invention.

The present invention relates to a nucleic acid amplification system comprising: a first primer and a second primer for amplifying a target DNA sequence, a DNA-dependent DNA polymerase, and dNTP monomers comprising a first label. Preferably, the system further comprises a recombinase. Preferably, the system further comprises an oligonucleotide probe comprising a nucleic acid sequence complementary to the target DNA sequence and a second label.

The present invention relates to a nucleic acid detection system for detecting a target DNA sequence comprising: a recombinase, an oligonucleotide probe comprising a nucleic acid sequence complementary to the target DNA sequence and a second label, and a nucleic acid amplification product comprising the target DNA sequence and comprising nucleotides comprising a first label.

The present invention relates to a nucleic acid amplification system comprising: a first primer and a second primer for amplifying a target DNA sequence, wherein the second primer comprises a second label and hybridizes to a first strand of DNA of the target DNA sequence, and a DNA-dependent DNA polymerase. Preferably, the system further comprises a recombinase.

Preferably, the system further comprises an oligonucleotide probe comprising a nucleic acid sequence complementary to the target DNA sequence and one or more nucleotides comprising a first label, preferably wherein the probe hybridizes to the first strand or the second strand of DNA of the target DNA sequence, the second strand being complementary to the first strand.

The present invention relates to a nucleic acid detection system for detecting a target DNA sequence comprising: a recombinase, an oligonucleotide probe comprising a nucleic acid sequence complementary to the target DNA sequence and one or more nucleotides comprising a first label, a nucleic acid amplification product comprising the target DNA sequence, and a primer comprising a second label and hybridized to a strand of DNA of the target DNA sequence.

The present invention relates to a lateral flow device, comprising a lateral flow strip, comprising:
a sample application zone:
a reagent zone downstream of the sample application zone and in fluid communication with the sample application zone, wherein the reagent zone comprises a non-immobilized first binding agent that binds to a first label;
a test zone downstream of the reagent zone and in fluid communication with the reagent zone, wherein the test zone comprises an immobilized second binding agent that binds to a second label; and
a control zone downstream of the test zone and in fluid communication with the test zone.
   Preferably, the reagent zone comprises a dried, preferably lyophilized, composition for amplifying a target DNA sequence. Preferably, the composition is a composition for RPA. Preferably, the composition is the first composition or the second composition according to the invention.

The invention also relates to a first kit of parts comprising dNTP monomers comprising a first label and an oligonucleotide probe comprising a nucleic acid sequence complementary to a target DNA sequence and a second label. Preferably, the kit of parts is for amplification of a nucleic acid, preferably wherein the amplification is recombinase polymerase amplification (RPA). Preferably, the kit of parts comprises a recombinase. Preferably, the kit of parts comprises a DNA-dependent polymerase. Preferably, the kit of parts comprises a first primer and a second primer for amplifying a target DNA sequence. Preferably, the kit of parts comprises a lateral flow device or the device according to the invention. The first kit of parts according to the invention may further comprise features of the first method of the invention or features of the second method of the invention in so far possible.

The invention further relates to a second kit of parts comprising a first primer and a second primer for amplifying a target DNA sequence, wherein the second primer comprises a second label and hybridizes to a first strand of DNA of the target DNA sequence; and an oligonucleotide probe comprising a nucleic acid sequence complementary to the target DNA sequence and one or more nucleotides comprising a first label, preferably wherein the probe hybridizes to the first strand or the second strand of DNA of the target DNA sequence, the second strand being complementary to the first strand. Preferably, the kit of parts is for amplification of a nucleic acid, preferably wherein the amplification is recombinase polymerase amplification (RPA). Preferably, the kit of parts comprises a recombinase. Preferably, the kit of parts comprises a DNA-dependent polymerase. Preferably, the kit of parts comprises a lateral flow device or the device according to the invention. The second kit of parts according to the invention may further comprise features of the second method of the invention or features of the first method of the invention in so far possible.

Preferably, the kit of parts according to the invention is nuclease-free or protease-free. Preferably, the kit of parts is nuclease-free and protease-free. Suitably, the kit of parts is ATP-free or ATP-analogue-free, preferably comprising DrRecA. Optionally, the kit of parts according to the invention comprises instructions for carrying out the method according to the invention. Preferably, the kit of parts according to the invention is for use in the method according to the invention.

In addition, the present invention relates to a use of the device, system, or kit of parts according to the invention for determining the presence or absence of a pathogen or a target DNA sequence associated with a disease or disorder or the onset thereof. Preferably, the pathogen is a human, animal, or plant pathogen. Preferably, the disease or disorder or the onset thereof comprises cancer, autoimmune disease or disorder, hereditary disease or disorder, pain, respiratory disease or disorder, cardiovascular disease or disorder, infectious disease or disorder, thyroid disease or disorder, skin disease or disorder, diabetes, obesity, kidney disease or disorder, gastrointestinal disease or disorder, neurodegenerative disease or disorder, musculoskeletal disease or disorder, metabolic disease or disorder, or mental illness, more preferably cancer, autoimmune disease or disorder, or infectious disease or disorder. Preferably, the target DNA sequence associated with a disease or disorder or the onset thereof comprises a DNA sequence expressing tumour antigens, tumour markers, or tumour suppressors. Preferably, the target DNA sequence associated with a disease or disorder or the onset thereof comprises a DNA sequence having a nucleotide sequence polymorphism. Preferably, the nucleotide sequence polymorphism comprises one or more somatic mutations and/or single nucleotide polymorphisms. Preferably, the target DNA sequence associated with a disease or disorder or the onset thereof comprises a DNA sequence associated with an epigenetic event, preferably DNA methylation and/or DNA hydroxymethylation.

The device, system, or kit of parts according to the invention can be used for determining the presence or absence of a target DNA sequence in various types of samples. For example, these include, but are not limited to a diagnostic sample, a veterinary sample, a pharmaceutical sample, a cosmetics sample, a food sample, a beverage sample, a feed sample, an agricultural sample, a soil sample, an air sample, a water sample, a waste sample, an effluent sample, or a sewage sample.

The present invention relates to a use of the device, system, or kit of parts according to the invention for determining the presence or absence of a target DNA sequence in a diagnostic sample, a veterinary sample, a pharmaceutical sample, a cosmetics sample, a food sample, a beverage sample, a feed sample, an agricultural sample, a soil sample, an air sample, a water sample, a waste sample, an effluent sample, or a sewage sample.

### DETAILED DESCRIPTION OF THE FIGURES

The invention will now be discussed with reference to the figures, which show preferred exemplary embodiments of the subject invention.

FIG. 1 shows a schematic depiction of the first method of the invention. A sample comprising a DNA molecule (100), here comprising a target DNA sequence, is provided for determining the presence or absence of a target DNA sequence. The sample is contacted with: a first primer (101) and a second primer (102) for amplifying the target DNA sequence; a recombinase (not shown); a DNA-dependent DNA polymerase (not shown); dNTPs comprising a first label (103); and an oligonucleotide probe (104) comprising a nucleic acid sequence complementary to the target DNA sequence and a second label (105). Optionally, the probe comprises a polymerase blocking group (106), preferably at the 3′ end of the probe as shown in FIG. 1. The blocking group (106) prevents polymerase extension of the primer. The target DNA sequence is amplified (107) by e.g. PCR or RPA to produce a nucleic acid amplification product (110), wherein dNTPs comprising a first label (103) have been incorporated in the nucleic acid amplification product (110). The probe (104) and the recombinase may also be added to the sample after amplification (107), which can be their only addition or in addition to the first addition. The recombinase facilitates formation of a DNA triplex (111) between the target DNA sequence and the probe. The nucleic acid amplification product is subsequently detected via for example flocculation (108), lateral flow strip (109), or ELISA (not shown).

Briefly, detection via flocculation (108) comprises combining the nucleic acid amplification product (110) and a first binding agent (112) capable of binding the first label, wherein the product and the first binding agent are capable of forming a complex which can be detected visually, wherein the complex is formed by flocculation, preferably by flocculation of the first binding agent. The first binding agent preferably binds specifically to the first label. Here, if the first label is biotin, the first binding agent (112) can be streptavidin-coated gold nanoparticles, the flocculation of which enables visual detection of a nucleic acid amplification product because of the large amount of incorporated first label.

Briefly, detection via lateral flow strip (109) comprises providing a lateral flow strip comprising a first binding agent (112) capable of binding the first label and a second binding agent (115) capable of binding the second label, preferably wherein the second binding agent is immobilized on the lateral flow strip. The lateral flow strip typically comprises a nitrocellulose membrane (116) and an end zone (118). The nucleic acid amplification product (110) is applied to the sample application zone (113) of the lateral flow strip. The product moves along the strip, passing the reagent zone (114) comprising the first binding agent (112) capable of binding the first label upon which the first binding agent binds to the first label incorporated in the nucleic acid amplification product. The product moves further along the strip to a test zone where it binds to the immobilized second binding agent (115) capable of binding the second label if the probe (104) has been bound to the product. This then enables detection of the nucleic acid amplification product. A control zone comprising a binding agent (117) capable of binding the first binding agent binds remaining first binding agent. Here, if the first label is biotin, the first binding agent (112) can be streptavidin-coated gold nanoparticles. Binding of streptavidin-coated gold nanoparticles to the large amount of incorporated first label enables visual detection at a high sensitivity. The probe (114) bound to the product enables a high specificity of the detected product.

FIG. 2 shows a schematic depiction of the second method of the invention. A sample comprising a DNA molecule (100), here comprising a target DNA sequence, is provided for determining the presence or absence of a target DNA sequence. The sample is contacted with: a first primer (101) and a second primer (102) for amplifying the target DNA sequence, wherein the second primer comprises a second label (105); a recombinase (not shown); a DNA-dependent DNA polymerase (not shown); dNTPs; and an oligonucleotide probe (104) comprising a nucleic acid sequence complementary to the target DNA sequence and one or more nucleotides comprising a first label (103). Optionally, the probe comprises a polymerase blocking group (106), preferably at the 3′ end of the probe as shown in FIG. 2. The blocking group (106) prevents polymerase extension of the primer. The target DNA sequence is amplified (107) by e.g. PCR or RPA to produce a nucleic acid amplification product (110). The probe (104) and the recombinase may also be added to the sample after amplification (107), which can be their only addition or in addition to the first addition. The recombinase facilitates formation of a DNA triplex (111) between the target DNA sequence and the probe. The nucleic acid amplification product is subsequently detected via for example flocculation (108), lateral flow strip (109), or ELISA (not shown).

Briefly, detection via flocculation (108) comprises combining the nucleic acid amplification product (110) and a first binding agent (112) capable of binding the first label, wherein the product and the first binding agent are capable of forming a complex which can be detected visually, wherein the complex is formed by flocculation, preferably by flocculation of the first binding agent. The first binding agent preferably binds specifically to the first label. Here, if the first label is biotin, the first binding agent (112) can be streptavidin-coated gold nanoparticles, the flocculation of which enables visual detection of a nucleic acid amplification product because of the large amount of first label bound to the product via the probe.

Briefly, detection via lateral flow strip (109) comprises providing a lateral flow strip comprising a first binding agent (112) capable of binding the first label and a second binding agent (115) capable of binding the second label, preferably wherein the second binding agent is immobilized on the lateral flow strip. The lateral flow strip typically comprises a nitrocellulose membrane (116) and an end zone (118). The nucleic acid amplification product (110) is applied to the sample application zone (113) of the lateral flow strip. The product moves along the strip, passing the reagent zone (114) comprising the first binding agent (112) capable of binding the first label upon which the first binding agent binds to the first label incorporated in the probe (104) if the probe has been bound to the product. The product moves further along the strip to a test zone where it binds to the immobilized second binding agent (115) capable of binding the second label of the second primer (102). This then enables detection of the nucleic acid amplification product. A control zone comprising a binding agent (117) capable of binding the first binding agent binds remaining first binding agent. Here, if the first label is biotin, the first binding agent (112) can be streptavidin-coated gold nanoparticles. Binding of streptavidin-coated gold nanoparticles to the large amount of incorporated first label enables visual detection at a high sensitivity. The probe (114) bound to the product enables a high specificity of the detected product.

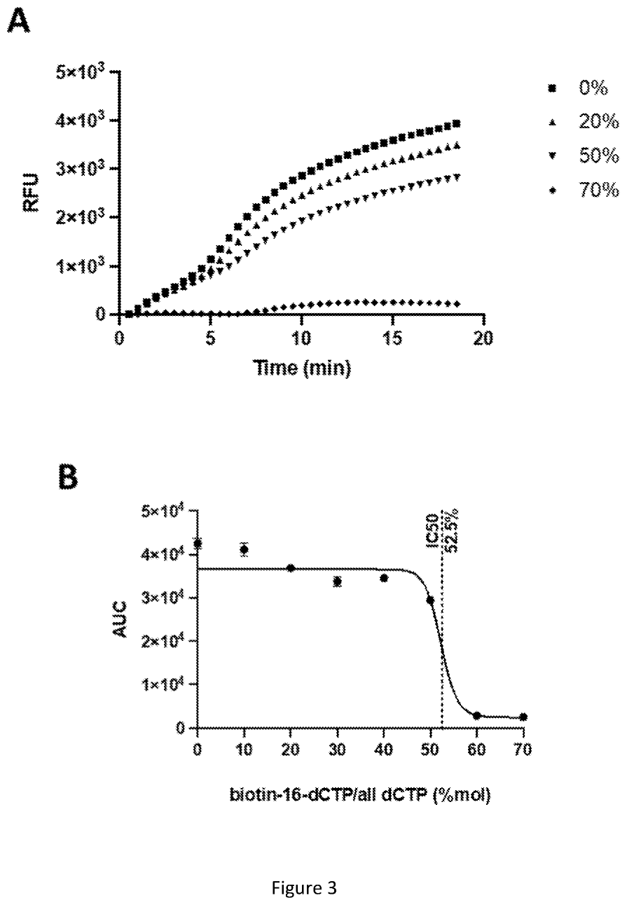

FIG. 3 shows the effect of biotin-dCTP concentration on RPA yield. The effect of biotin-16-dCTP incorporation during recombinase polymerase amplification was assessed using a real-time assay. FIG. 3A shows amplification with different biotin-16-dCTP concentrations of total dCTP used (mol %), namely 0%, 20%, 50%, and 70% moles of biotin-16-dCTP of total dCTP. Fluorescence, indicating double-stranded DNA formation, was measured every 30 sec. The y-axis indicates relative fluorescence, the x-axis indicates time in minutes. Further details of the experiment are described in Example 1. The maximum tolerable amount was found to be around 50% moles of biotin-16-dCTP over total dCTP. FIG. 3B shows dose-response of % biotin-16-dCTP vs area under the curve (AUC) of amplification curves of FIG. 3A. The y-axis indicates AUC, the x-axis indicates different percentages of biotin-16-dCTP of total dCTP in % mol. A half-maximal response (IC50) was found at 52.5%.

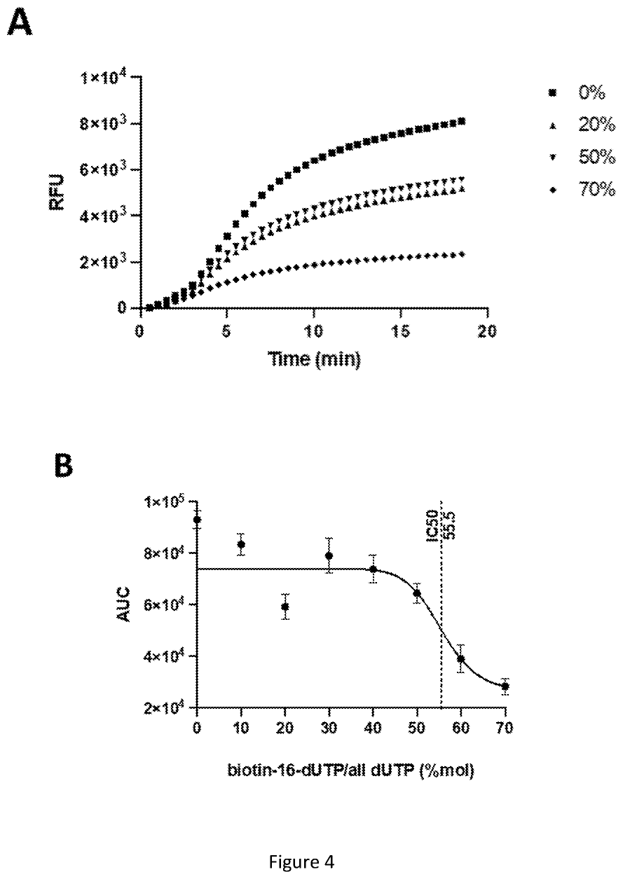

FIG. 4 shows the effect of biotin-dUTP concentration on RPA yield. The effect of biotin-16-dUTP incorporation during recombinase polymerase amplification was assessed using a real-time assay. FIG. 4A shows amplification with different biotin-16-dUTP concentrations of total dUTP used (mol %), namely 0%, 20%, 50%, and 70% moles of biotin-16-dUTP of total dUTP. Fluorescence, indicating double-stranded DNA formation, was measured every 30 sec. The y-axis indicates relative fluorescence, the x-axis indicates time in minutes. Further details of the experiment are described in Example 2. The maximum tolerable amount was found to be around 50% moles of biotin-16-dUTP over total dUTP. FIG. 4B shows dose-response of % biotin-16-dUTP vs area under the curve (AUC) of amplification curves of FIG. 4A. The y-axis indicates AUC, the x-axis indicates different percentages of biotin-16-dUTP of total dUTP in % mol. A half-maximal response (IC50) was found at 55.5%.

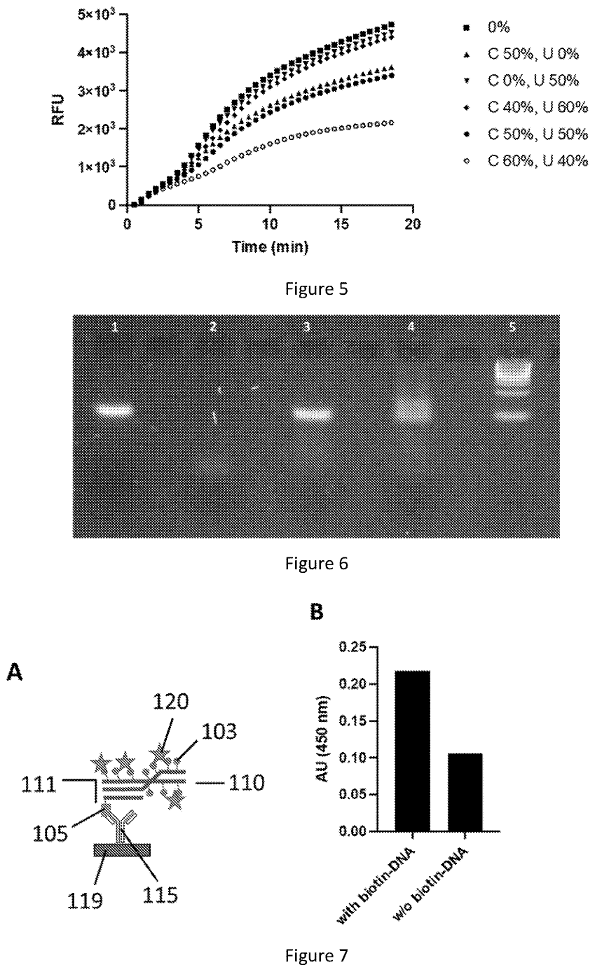

FIG. 5 shows the effect of the effect of both biotin-dCTP and biotin-dUTP concentration on RPA yield. The effect of biotin-16-dCTP and biotin-16-dUTP incorporation during recombinase polymerase amplification was assessed to maximize the amount of biotin per amplicon using a real-time assay. FIG. 5 shows real-time RPA amplification of six different conditions, where respectively 0%, 50% C and 0% U, 0% U and 50% C, 40% C and 60% U, 50% C and 50% U, and 60% C and 40% U were used, n=2. “C” indicates mol/mol of biotin-16-dCTP from total dCTP in a single reaction and “U” indicates mol/mol biotin-16-dUTP of total dUTP in a single reaction. Fluorescence, indicating double-stranded DNA formation, was measured every 30 sec. The y-axis indicates relative fluorescence, the x-axis indicates time in minutes. Further details of the experiment are described in Example 3. The maximum yield was observed when using 50% biotin-16-dUTP and the combination of 40% biotin-16-dCTP and 60% biotin-16-dUTP.

FIG. 6 shows evidence of D-loop formation on agarose gel. FIG. 6 shows the result of reactions containing different components: lane 1 contains homologous DNA only, lane 2 contains primer only, lane 3 contains homologous DNA, primer and RecA, lane 4 contains homologous DNA, primer, RecA, and ATPγS, lane 5 contains a 100 bp DNA ladder, where the bottom band indicates 100 bp and the band above the bottom band indicates 200 bp. Homologous DNA contains a sequence complementary to the primer, which is here used as a probe according to the invention. Further details of the experiment are described in Example 5. The non-denaturing agarose gel shows the formation of a stable D-loop in the presence of ATPγS, a primer, RecA, and homologous DNA (lane 4). ATPγS is known to stabilize D-loop which was observed as a smear in lane 4.

FIG. 7 shows evidence of D-loop formation in ELISA. A high-throughput D-loop ELISA detection method that mimicks the layout of a lateral flow was developed, the details of which are described in Example 6. FIG. 7A illustrates the concept of the ELISA. Here, an antibody (115), i.e. a second binding agent according to the invention, is immobilized that binds a 5′-label (105) of a probe, the label being a second label according to the invention. A nucleic acid amplification product (110) comprising nucleotides having a first label (103) and produced according to the first method of the invention has been contacted with a recombinase and the probe. A D-loop (111) is subsequently formed, and the complex comprising the nucleic acid amplification product (110) and the probe is incubated with the immobilized antibody (115). After several customary washing steps, the bound complex is contacted with a streptavidin-horseradish peroxidase (HRP) fusion (120) and subsequently detected.

FIG. 7B shows an experiment using the concept illustrated in FIG. 7A. The left bar shows absorbance where homologous DNA containing 50% mol biotin-16-dUTP was used, while the right bar shows absorbance where homologous DNA was replaced by H₂O. The y-axis indicates absorbance at a wavelength of 450 nm. Homologous DNA contains a sequence complementary to the primer, which is here used as a probe according to the invention. A twofold increase in signal was observed in the presence of homologous biotinylated DNA.

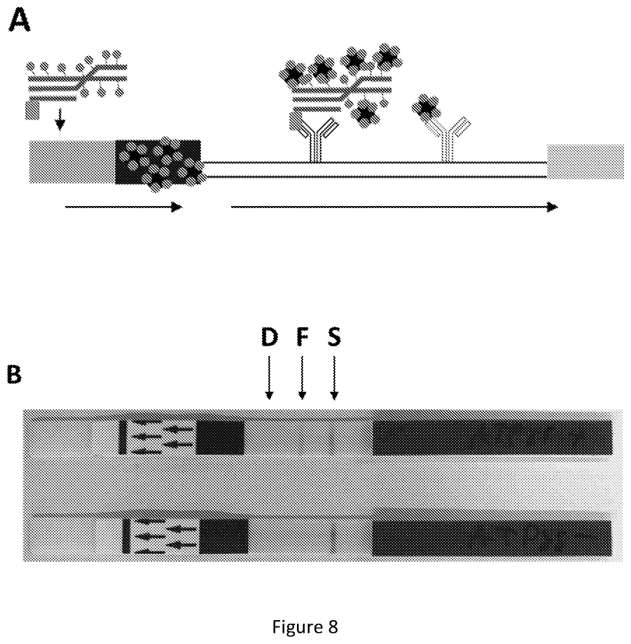

FIG. 8 shows evidence of D-loop formation in a lateral flow assay. The possibility of DNA detection on a lateral flow strip using a stable D-loop formation was assessed. The concept of the detection is illustrated in FIG. 8A with reference to FIG. 1 where the same symbols are used. A nucleic acid amplification product containing incorporated nucleotides comprising a first label has been contacted with a probe comprising a second label (i.e. FAM) and recombinase. The resulting complex containing a D-loop is subsequently contacted with a lateral flow strip and detected at the FAM binding site.

FIG. 8B shows a comparison of using ATPγS vs H₂O during contacting a nucleic acid amplification product with a probe comprising a second label being FAM and a recombinase. ATPγS is known to stabilize D-loops in combination with recombinase. “D” indicates the digoxigenin binding site, “F” indicates the FAM binding site, “S” indicates the streptavidin binding site. The top strip shows the result of an experiment using ATPγS, while the bottom strip shows the result of an experiment where ATPγS was replaced with H₂O. Further details of the experiment are described in Example 7. The band on the FAM binding site of the top strip indicates that ATPγS increases detection of a complex comprising the nucleic acid amplification product and the probe.

FIG. 9 shows effect of probe design on lateral flow assay. The stability of D-loop formation in various probe designs as a function of their design was assessed, wherein design refers to a probe that binds to either a terminal end of the biotinylated homologous DNA (FIGS. 9A and B) or to the middle (FIGS. 9C, D, E and F), wherein a 25 nucleotide (nt) probe was assessed in FIGS. 9C and D and a 60 nt probe was assessed in figures E and F. Homologous DNA contains a sequence complementary to the probe. In addition, several conditions were tried which are known to stabilize the D-loop since the D-loop that forms at the terminal end (i.e. plectonemic joint) is more stable than a D-loop that forms in the middle of the DNA duplex (i.e. paranemic joint). Those conditions were the presence of single-strand binding protein, PEG, KCl, trehalose, ionic liquid 1-ethyl-3-methylimidazolium acetate, and their combination. The results show that terminal probe binds regardless of the conditions whereas the binding of the middle-binding probe is a function of its length and the additive. The best result was achieved with 60 nt probe in the presence of the ionic liquid, see FIG. 9F. Further details of the experiments are described in Example 8.

FIGS. 9A, C, and E show schematic illustrations of the detection concepts of the different probes with reference to reference to FIG. 1 where the same symbols are used. A nucleic acid amplification product containing incorporated nucleotides comprising a first label has been contacted with a probe comprising a second label (i.e. digoxigenin) and recombinase, and optionally other additives. The resulting complex containing a D-loop is subsequently contacted with a lateral flow strip and detected at the digoxigenin binding site.

FIGS. 9B, D, and F show the results of experiments of the respective concepts of FIGS. 9A, C, and E in a lateral flow assay. “D” indicates the digoxigenin binding site, “F” indicates the FAM binding site, “S” indicates the streptavidin binding site. Strip 1 indicates no additive, strip 2 indicates added single-strand binding protein, strip 3 indicates added trehalose, PEG 6000 and KCl, strip 4 indicates added ionic liquid 1-ethyl-3-methylimidazolium acetate, strip 5 indicates added single-strand binding protein, trehalose, PEG, KCl, and ionic liquid 1-ethyl-3-methylimidazolium acetate. Further details of the experiments are described in Example 8. The strips of FIGS. 9B and F, respectively the terminal probe and the middle 60 nt probe, show bands in each of the digoxigenin binding sites, indicating detection of a complex comprising the nucleic acid amplification product and the probe.

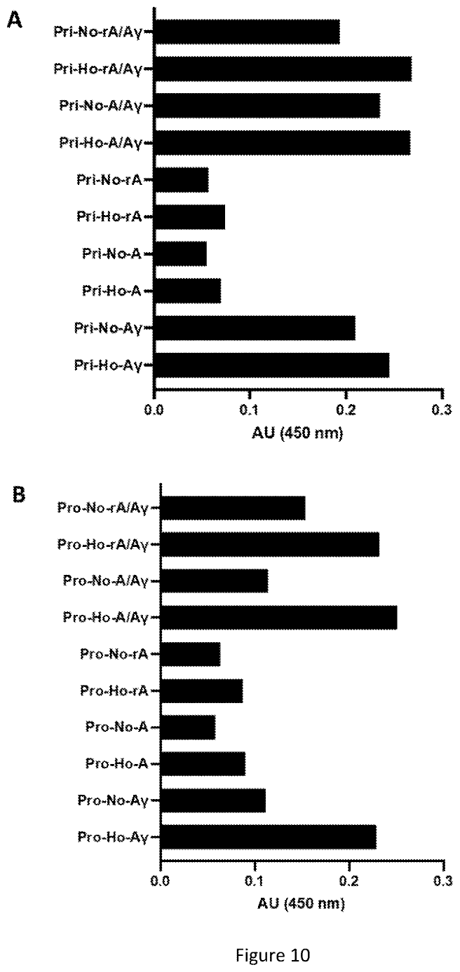

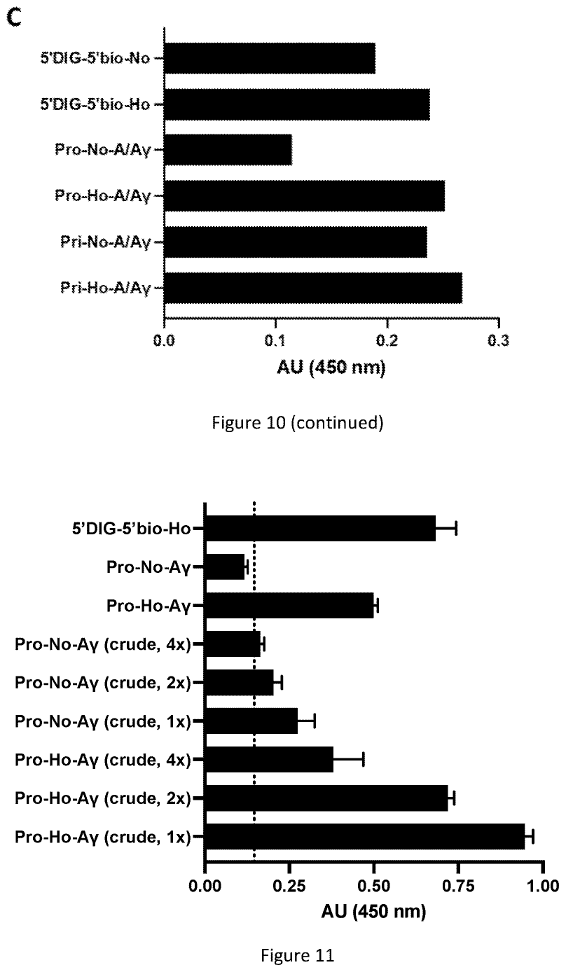

FIG. 10 shows specificity of primer-probe and probe with and without ATP-gamma-S, and also compared to a conventional hapten-based strategy. The extent of sequence-specific binding for the 29 nt primer-probe that binds to the terminal end of the amplicon of RPA-amplified and purified templates with 50% biotin-16-dUTP and the 60 nt probe that binds in the middle of the amplicon of RPA-amplified and purified templates with 50% biotin-16-dUTP was assessed. A comparison was also made with RPA amplification with conventional hapten-labelled primers. The influence of the replacement ATP-gamma-S with a finite amount of ATP and the ATP with ATP regeneration system (phosphocreatine and creatine kinase), and their combination with ATP-gamma-S was drawn.

FIG. 10A shows the specificity of primer-probe-based (plectonemic joint) D-loop detection using ELISA. It shows the result of an experiment, wherein either homologous DNA template was amplified using suitable primers, resulting in the reference “Ho” or wherein different non-homologous DNA template was amplified using suitable primers, resulting in the reference “No”. The amplified DNA was subjected to the primer-probe (indicated by “Pri”) and recombinase incubation using different ATP/ATPγS inputs. Here, homologous and non-homologous refer to complementary sequence with the primer-probe. The different ATP/ATPγS inputs are: ATP regeneration via ATP with creatine kinase and phosphocreatine (1×Basic E-mix), indicated by “rA”; ATPγS only, indicated by “Aγ”; ATP only, indicated by “A”; a combination of rA and Aγ, indicated by “rA/Aγ”; a combination of A and Aγ, indicated by “A/Aγ”. The resulting D-loop was subsequently detected using ELISA.

Further details of the experiments are described in Example 9.

As the primer-probe is non-complementary to the non-homologous DNA template No, no D-loop should be formed, thereby resulting in a signal that can be considered a negative signal. On the contrary, the primer-probe is complementary to the DNA template Ho, resulting in a D-loop and a positive signal. It can be observed in FIG. 10A that for every ATP/ATPγS input condition the positive signal is higher than the negative signal, confirming the presence of a D-loop. A larger difference between a negative signal and a positive signal of a respective ATP/ATPγS input condition can be considered a greater specificity of the probe for the respective condition. Hence, the greatest specificity in FIG. 10A can be observed for the (Pri-)rA/Aγ condition, while the lowest specificity can be observed for the (Pri-)A condition. In general, a higher specificity can be observed when ATPγS is used.

FIG. 10B shows the specificity of probe-based (paranemic joint) D-loop detection using ELISA. It shows the result of an experiment, wherein either homologous DNA template was amplified using suitable primers, resulting in the reference “Ho” or wherein different non-homologous DNA template was amplified using suitable primers, resulting in the reference “No”. The amplified DNA was subjected to the 60 nt probe (indicated by “Pro”) and recombinase incubation using different ATP/ATPγS inputs. Here, homologous and non-homologous refer to complementary sequence with the primer-probe. The different ATP/ATPγS inputs are: ATP regeneration via ATP with creatine kinase and phosphocreatine (1×Basic E-mix), indicated by “rA”; ATPγS only, indicated by “Aγ”; ATP only, indicated by “A”; a combination of rA and Aγ, indicated by “rA/Aγ”; a combination of A and Aγ, indicated by “A/Aγ”. The resulting D-loop was subsequently detected using ELISA. Further details of the experiments are described in Example 9.

As the probe is non-complementary to the non-homologous DNA template No, no D-loop should be formed, thereby resulting in a signal that can be considered a negative signal. On the contrary, the probe is complementary to the DNA template Ho, resulting in a D-loop and a positive signal. It can be observed in FIG. 10B that for every ATP/ATPγS input condition the positive signal is higher than the negative signal, confirming the presence of a D-loop. A larger difference between a negative signal and a positive signal of a respective ATP/ATPγS input condition can be considered a greater specificity of the probe for the respective condition. Hence, the greatest specificity in FIG. 10B can be observed for the (Pro-)A/Aγ condition, while the lowest specificity can be observed for the (Pro-)rA condition. In general, a higher specificity can be observed when ATPγS is used.

Furthermore, comparing the results of the experiments of FIG. 10A with FIG. 10B it can be observed that probe-based D-loop detection has a greater specificity than primer-probe-based D-loop detection. This is surprising since the D-loop that forms at the terminal end (i.e. plectonemic joint) is more stable than a D-loop that forms in the middle of the DNA duplex (i.e. paranemic joint). FIG. 10C shows a comparison of specificity of D-loop detection with conventional dual-hapten labelled RPA amplicon using ELISA. The conditions Pro-No-A/Aγ, Pro-Ho-A/Aγ, Pri-No-A/Aγ, and Pri-Ho-A/Aγ are taken from the experiments as described in FIG. 10B and FIG. 10A. In addition, two conventional dual-hapten labelled RPA amplicons were generated as follows: the homologous and non-homologous DNA templates were each separately amplified using a biotin-labeled forward primer and a digoxigenin-labeled reverse primer resulting in the references “5′ DIG-5′ bio-Ho” (a specific amplification product of RPA) and “5′ DIG-5′ bio-No” (i.e. a nonspecific amplification product of RPA) respectively. The resulting product was subsequently detected using ELISA under the same conditions as the experiments described in FIGS. 10A and B. Further details of the experiments are described in Example 9.

It can be observed that D-loop detection according to the present invention, in particular when a (stable) paranemic joint is formed, results in an improved signal compared to detection of RPA amplified DNA by conventional dual-hapten labels, which results in a lower relative signal (i.e. the signal of 5′ DIG-5′ bio-Ho subtracted by the signal of 5′ DIG-5′ bio-No).

FIG. 11 shows that an RPA amplicon can be added directly into RecA-probe binding immobilized D loop detection with high reproducibility and specificity. Serial dilutions of a crude RPA reaction in deionized water were added in 1 microL volumes to RecA reactions in triplicate with 0.3 mM ATPγS and Probe_2, and subsequently added to ELISA in triplicate (Pro-Ho-Aγ, crude, [dilution factor 1×, 2×, or 4×]). A negative control (Pro-No-Aγ) and positive controls (5′ DIG-5′ bio-Ho and Pro-Ho-Aγ) were also used. As a test for specificity, Non_1 was amplified in RPA with 30% biotin-16-dUTP followed by the serial dilution of the crude RPA reaction in deionized water. The serial dilutions were added in 1 microL volumes to RecA reactions in triplicate with 0.3 mM ATPγS and Probe_2, and subsequently added to ELISA in triplicate (Pro-No-Aγ, crude, [dilution factor 1×, 2×, or 4×]). The comparison between the serial dilutions of Hom_1 and Non_1 amplicons in crude RPA reactions shows that the specificity and reproducibility of the D-loop based detection method is retained even after the direct addition of crude RPA product into RecA reaction. Further details of the experiment are described in Example 10.

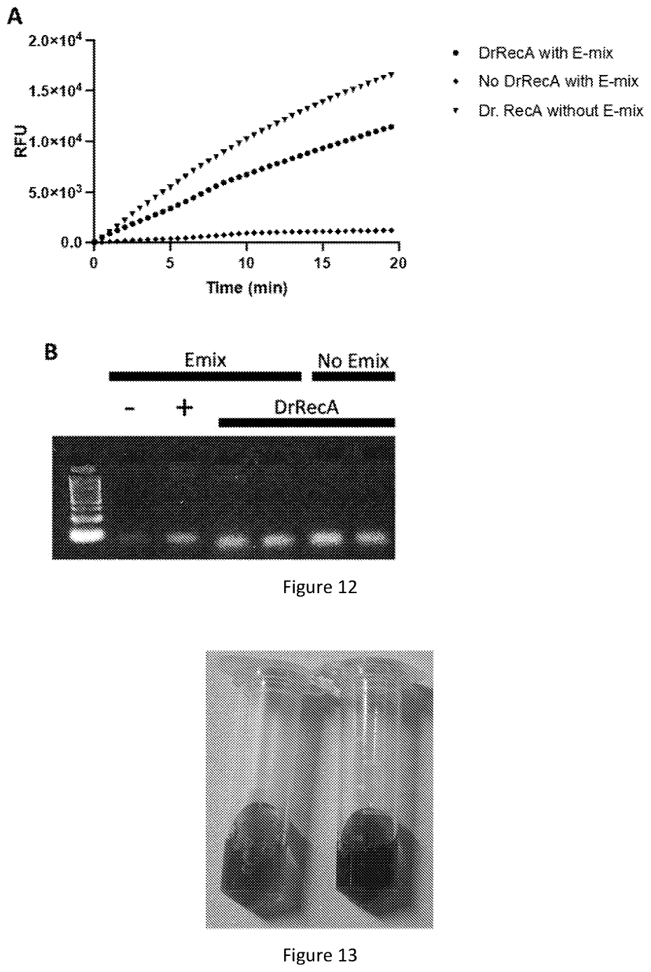

FIG. 12 shows that DrRecA does not require ATP or ATPγS for strand exchange. A real-time fluorescent assay was used for recombinase's strand exchange activity (FIG. 12A). Strand exchange activity was detected for RecA enzyme from Deinococcus radiodurans (DrRecA) and was not dependent on the presence of the ATP source. This was confirmed by comparing the fluorescence intensity of the real-time fluorescent assay products on an agarose gel (FIG. 12B). The presence of fluorescence upon the addition of DrRecA without ATP source indicates strand exchange without the necessity for ATP and its cofactors, which may simplify the reaction composition of RecA reaction. Further details of the experiment are described in Example 11.

FIG. 13 shows that bridge flocculation allows naked-eye detection of biotinylated DNA. Bridge flocculation was performed on samples comprising a purified RPA product comprising biotinylated DNA amplicons according to the invention. A distinct difference in coloration between the samples treated with the purified RPA product with and without template DNA can be observed and indicates that this method can be used to detect biotinylated RPA amplicons with a naked eye after RPA. Further details of the experiment are described in Example 12.

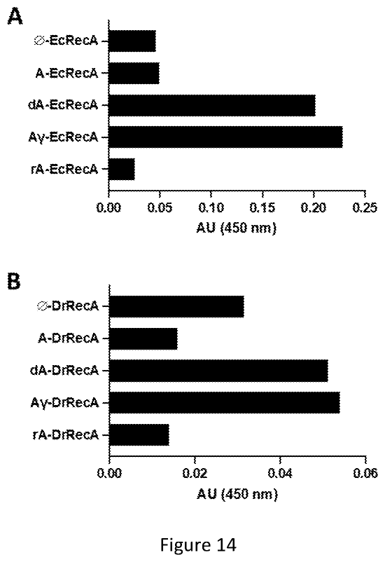

FIG. 14 shows stable D-loop formation by EcRecA and DrRecA. Using ELISA, stable D-loop formation was analysed using different ATP compositions: no ATP (0), ATP (A), dATP (dA), ATPγS (Aγ), and ATP regeneration system (rA). Further details of the experiment are described in Example 13.

Throughout the description and claims of this specification, the words “comprise” and “contain” and variations of them mean “including but not limited to”, and they are not intended to (and do not) exclude other moieties, additives, components, integers, or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties, or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Aspects of the invention are demonstrated by the following non-limiting examples.

### EXAMPLES

### General Materials and Methods

The following materials and methods were used in the examples, unless stated otherwise.

The methods of the examples are nuclease-free and protease-free: no nucleases or proteases were used.

### Materials

Primers (see Table 1), probes (see Table 2) and synthetic target and non-target double stranded DNA templates (see Table 3) were procured from IDT and Biolegio. TwistDx Liquid Kit was purchased from TwistDx. PCR reaction components, RecA, RecA Reaction Buffer, T4 Gene 32 Protein, Gel Loading Dye, Purple (6×, no sodium dodecyl sulphate (SDS)) and 100 bp DNA Ladder were acquired from New England Biolabs. ATP γ-S was purchased from Abcam. SYBR Safe, Qubit assays and E-Gel Power Snap Electrophoresis System were procured from ThermoFisher Scientific. Tissue culture (TC) treated flat-bottom transparent 96-well ELISA plates were acquired from Greiner. PCRD FLEX lateral flow strips and PCRD FLEX Extraction Buffer were supplied by Abington Health. Biotin-16-dCTP was procured from Jena Biosciences. Antidigoxin and antifluorescein antibodies were purchased from Enzo Life Sciences. Agarose, salts, 3,3′,5,5′-tetramethylbenzidine (TMB), dithiothreitol (DTT), H₂O₂, H₂SO₄, bovine serum albumin (BSA) and buffers were purchased from Fisher Scientific and Merck unless stated otherwise.

TABLE 1
Primers

| | SEQ | |
|---|---|---|
| Name | ID NO | Sequence (5′ to 3′) |
| Fwd_1 | 1 | CACTTGAGATTCTTGACATTACACC |
| (32 nt) | | ATGTTCT |
| Fwd_2 | 2 | CAGTGTATCTGGAAAGCCTACAGGA |
| (30 nt) | | CACCA |
| Rev_1 | 3 | AACAAATCTTATTTTCGAATACGTA |
| (29 nt) | | CACG |
| Rev_2 | 4 | TTGATACATTCGGTCTCGTGTATCT |
| (30 nt) | | TCTAG |
| 5′FAM- | 9 | [FAM]CACTTGAGATTCTTGACATT |
| Fwd_1 | | ACACCATGTTCT |
| (32 nt) | | |
| AntiFwd_ | 10 | AGAACATGGTGTAATGTCAAGAATC |
| 1-3′BHQ1 | | TCAAGTG[BHQ1] |
| (32 nt) | | |

TABLE 2
Probes

| | SEQ | |
|---|---|---|
| Name | ID NO | Sequence (5′ to 3′) |
| Probe_1 | 5 | AGTACTGATATGTCCCTGATTGGTG |
| (25 nt) | | |
| Probe_2 | 6 | ACACCATGTTCTGTAGTACTGATAT |
| (60 nt) | | GTCCCTGATTGGTGAACGTGTACGT |
| | | ATTCGAAAAT |

TABLE 3
Synthetic DNA templates

| | SEQ | |
|---|---|---|
| Name | ID NO | Sequence (5′ to 3′) |
| Hom_1 | 7 | ACAGACACTTGAGATTCTTGACATTACACCATG |
| (100 bp) | | TTCTGTAGTACTGATATGTCCCTGATTGGTGAA |
| | | CGTGTACGTATTCGAAAATAAGATTTGTTTCTC |
| | | G |
| Non_1 | 8 | CAGTGTATCTGGAAAGCCTACAGGACACCAAAA |
| (140 bp) | | TAACCTTAATCATCAATTGGTTACAGGAGGCTT |
| | | TAAGTTCAGCATCTTTGGCTCACATGAAGGCCA |
| | | AATTCCGAGAGACCCTAGAAGATACACGAGACC |
| | | GAATGTAT |

### Primer Design and Validation

Target and non-target DNA template sequences were selected in accordance with the requirements outlined by TwistDx: GO content between 40% and 60%, few repetitive sequences, and absence of direct/inverted repeats and palindromes. PrimedRPA (Higgins et al.), accessed through GitHub (Higgins, M. PrimedRPA, 2022) on 07.02.2022), was used to design primer pairs for the target sequences.

The resulting primer pair candidates were validated by performing RPA using TwistDx Liquid Basic Kit according to the manufacturer's protocol, purifying with Zymogen DNA Clean & Concentrator, and running on 4% agarose gel electrophoresis in 1×TBE buffer prestained with SYBR Safe, and visualized using E-Gel Power Snap Electrophoresis System.

Where indicated, the primer may have incorporated on its respective 5′ or 3′ nucleotide a 5′ digoxigenin or 5′ fluorescein (e.g. FAM) or 5′ biotin.

### Probe Design

The probe was designed such that it overlaps with the amplified sequence and/or the primer(s) (Piepenburg et al.), spans the GC content between 30% and 70%, and the length between 25 and 60 nts. Where indicated, the probe may have incorporated on its respective 5′ or 3′ nucleotide a 5′ digoxigenin, a 5′ fluorescein (e.g. FAM), an inverted 3′ dideoxythymidine (inverted_dT), and/or a 3′ C3 hydrocarbon spacer (C3).

The probe can also have the same sequence as one of the primers and hence can be termed a primer-probe.

The probe binding was qualitatively assessed by means of electromobility shift assay (Chen et al.) by incubating 3.2 pmol of probe with the 0.2 pmol of complementary double strand in the presence of 32 pmol of RecA and 0.3 mM ATP γ-S in 10 μl 1×RecA Reaction Buffer. If applicable, the molar ratio of probe to the target DNA was kept between 1:1 to 1:16. The molar ratio of RecA to probe was kept constant at 10:1. Optionally, the reaction was supplemented with 5% w/v trehalose (Ozay et al.), 10 mM KCl, 20% w/v PEG 6000 (Teng et al.), and/or ATP (10-fold molar excess compared to ATP γ-S) and/or T4 GP32 single-strand binding protein (SSB) (molar ratio of SSB to RecA between 2:1 and 3:1 (Riddles and Lehman)) and/or the ionic liquid 1-ethyl-3-methylimidazolium acetate (Martzy et al.). The reaction was incubated at 37 °C for 10 minutes and run on 4% agarose gel electrophoresis in 1×TBE buffer prestained with SYBR Safe to 1×, and visualized using E-Gel Power Snap Electrophoresis System.

### Example 1—the Effect of Biotin-dCTP Concentration on RPA Yield

### Methods

The following 25 μL RPA reaction was incubated at 42 °C for 20 min and its fluorescence was read every 30 sec in the SYBR channel of a real-time quantitative PCR machine (BioRad): 1× core mix (TwistDx), 1× reaction buffer (TwistDx), 1× basic E-mix (TwistDx), 480 nM primers (each of Fwd_1 (32 nt) and Rev_1 (29 nt)), 100 copies of homologous DNA (Hom_1 (100 bp)), 14 mM magnesium acetate, 0.08 mM dNTP (each), 10%-70% mol/mol biotin-16-dCTP (of total dCTP), 1×LAMP Fluorescent Dye (NEB). According to Munawar et al., core mix is composed of GP32 SSB, UvsX recombinase, UvsY and polymerase and the basic E-mix contains phosphocreatine, ATP, creatine kinase, high salt buffer and dH₂O.

### Results

The effect of biotin-16-dCTP incorporation during recombinase polymerase amplification to maximize the amount of biotin per amplicon was assessed using a real-time assay. The maximum tolerable amount was found to be around 50% moles of biotin-16-dCTP over total dCTP. The results are presented in FIG. 3. FIG. 3A shows real-time RPA amplification of four different conditions, where respectively 0%, 20%, 50%, and 70% moles of biotin-16-dCTP of total dCTP were used, n=2. Fluorescence, indicating double-stranded DNA formation, was measured every 30 sec. FIG. 3B shows dose-response of % biotin-16-dCTP vs area under the curve (AUC) of amplification curves of FIG. 3A, n=2.

### Example 2—the Effect of Biotin-dUTP Concentration on RPA Yield

### Methods

The following 25 μL RPA reaction was incubated at 42 °C for 20 min and its fluorescence was read every 30 sec in the SYBR channel: 1× core mix (TwistDx), 1× reaction buffer (TwistDx), 1× basic E-mix (TwistDx), 480 nM primers (each of Fwd_1 (32 nt) and Rev_1 (29 nt)), 100 copies of homologous DNA (Hom_1 (100 bp)), 14 mM magnesium acetate, 0.08 mM dNTP (each), 10%-70% mol/mol biotin-16-dUTP (of total dUTP), 1×LAMP (NEB).

### Results

The effect of biotin-16-dUTP incorporation during recombinase polymerase amplification to maximize the amount of biotin per amplicon was assessed using a real-time assay. The maximum tolerable amount was found to be around 50% moles of biotin-16-dUTP over total dUTP. The results are presented in FIG. 4. FIG. 4A shows real-time RPA amplification of four different conditions, where respectively 0%, 20%, 50%, and 70% moles of biotin-16-dUTP of total dUTP were used, n=2. Fluorescence, indicating double-stranded DNA formation, was measured every 30 sec. FIG. 4B shows dose-response of % biotin-16-dUTP vs area under the curve (AUC) of amplification curves of FIG. 4A, n=2.

### Example 3—the Effect of Both Biotin-dCTP and Biotin-dUTP Concentration on RPA Yield Methods

The following 25 μL RPA reaction was incubated at 42 °C for 20 min and its fluorescence was read every 30 sec in the SYBR channel: 1× core mix (TwistDx), 1× reaction buffer (TwistDx), 1× basic E-mix (TwistDx), 480 nM primers (each of Fwd_1 (32 nt) and Rev_1 (29 nt)), 100 copies of homologous DNA (Hom_1 (100 bp)), 14 mM magnesium acetate, 0.08 mM dNTP (each), 10%-70% mol/mol biotin-16-dCTP (of total dCTP) and biotin-16-dUTP (of total dUTP) (each), 1×LAMP (NEB).

### Results

The effect of biotin-16-dCTP and biotin-16-dUTP incorporation during recombinase polymerase amplification was assessed to maximize the amount of biotin per amplicon using a real-time assay. The maximum yield was observed when using 50% biotin-16-dUTP and the combination of 40% biotin-16-dCTP and 60% biotin-16-dUTP. The results are presented in FIG. 5. FIG. 5 shows real-time RPA amplification of six different conditions, where respectively 0%, 50% C and 0% U, 0% U and 50% C, 40% C and 60% U, 50% C and 50% U, and 60% C and 40% U were used, n=2. “C” indicates mol/mol of biotin-16-dCTP from total dCTP in a single reaction and “U” indicates mol/mol biotin-16-dUTP of total dUTP in a single reaction. Fluorescence, indicating double-stranded DNA formation, was measured every 30 sec.

### Example 4—Target Sequence DNA Amplification Using PCR or RPA

### Methods

### PCR Reaction

The PCR reaction mixes were assembled in 0.2 mL capped PCR tubes on ice and contained 1× Fast Start High Fidelity Reaction Buffer with 1.8 mM MgCl₂, 0.48 μM of each RPA primer, 1 ng DNA template (concentration was determined using Qubit dsDNA High Sense Assay Kit and Qubit fluorometer), 10% v/v DMSO, 0.44 mM dNTP (each), 0.44 mM biotin-16-dCTP, 1 unit of Fast Start Taq DNA polymerase and nuclease-free water in a total volume of 25 μL. The reaction mixes were mixed by brief vortexing and spun down. The PCR program was run in MJ Mini Personal Thermal Cycler (Bio-Rad) and consisted of (1) an initial denaturation step of 4 min at 95 °C; (2) 35 cycles of 30 sec at 95 °C, 30 sec at 49 °C, and 45 sec at 72 °C; (3) a final extension step of 7 min at 72 °C followed by quenching at 4 °C. The PCR products were purified using Zymogen DNA Clean & Concentrator and run on 4% agarose gel electrophoresis in 1×TBE buffer prestained with SYBR Safe, and visualized using E-Gel Power Snap Electrophoresis System.

### RPA Reaction

The RPA reaction mixes were assembled in 0.2 mL capped PCR tubes on ice and contained 1× Reaction Buffer, 1×E-mix Basic (containing creatine kinase, creatine phosphate, and ATP), 1× Core Reaction Mix, 0.48 μM of each RPA primer, 0.08 mM dNTP (each), 0.08 mM biotin-16-dCTP, 14 mM magnesium acetate, 50 copies DNA template and nuclease-free water in a total volume of 25 μL. The components of the reaction mixture were mixed with a pipette and template and magnesium were added last in separate volumes in the lid of the tube. The tube was spun down, mixed with a pipette on ice and incubated at 42 °C in MJ Mini Personal Thermal Cycler (Bio-Rad) for 20 min followed by quenching the reaction at 4 °C. The RPA products were purified using Zymogen DNA Clean & Concentrator and run on 4% agarose gel electrophoresis in 1×TBE buffer prestained with SYBR Safe to 1×, and visualized using E-Gel Power Snap Electrophoresis System.

### Binding of PCR or RPA Amplicon to a Probe

The crude PCR or RPA reaction was then further used in examples 4 to 8.

### PCR or RPA Amplicon Detection Using Bride Flocculation

The 25 μL of a crude PCR or RPA reaction is diluted to 100 uL with 0.05 mg/mL DiagNano PEG-streptavidin silica-coated gold nanoparticles of 40 nm in diameter and silica shell of 3 nm (CD Bioparticles) in 1×PBS, 0.05% Tween-20 and 1% BSA. The solution is incubated at at 25 °C for 15 min. The absorbance of the solution is measured in TC-treated flat-bottom transparent 96-well ELISA plate at the wavelengths of 520 nm and 650 nm (D'Agata et al.).

### Results

An increased absorbance can be observed with increasing concentrations of PCR or RPA amplified product.

### Example 5—Evidence of D-Loop Formation on Agarose Gel

### Methods

In a 0.2 mL PCR tube, mixed 0.8 pmol of homologous DNA ((Hom_1 (100 bp) containing 50% mol biotin-16-dCTP), 12.8 pmol forward primer (Fwd_1 (32 nt)), and 128 pmol RecA in 10 μL of 0.3 mM ATPγS, 70 mM Tris-HCl, 10 mM MgCl₂, and 5 mM DTT pH 7.6. The mixture was incubated at 37 °C for 10 min. The 2 μL of Gel Loading Dye, Purple (6×) (NEB) was added and the sample was run at 28 V in Tris-Borate-EDTA (TBE) buffer (1×) for 30 min on a 4% w/v agarose gel stained with SYBR Safe (1×) (ThermoFisher).

The above method is based on NEB's protocol “Using RecA and an oligonucleotide to form a stable triple helix” (https://international.neb.com/protocols/0001/01/01/using-reca-and-an-oligonucleotide-to-form-a-stable-triple-helix, accessed on Jan. 8, 2022).

### Results

A non-denaturing agarose gel shows the formation of a stable D-loop in the presence of ATPγS, a primer, RecA, and homologous DNA (FIG. 6, lane 4). The presence and absence of ATPγS was used for comparison as ATPγS is known to stabilize D-loop which was observed as a smear in lane 4. FIG. 6 shows the result of reactions containing different components: lane 1 contains homologous DNA only, lane 2 contains primer only, lane 3 contains homologous DNA, primer and RecA, lane 4 contains homologous DNA, primer, RecA, and ATPγS, lane 5 contains a 100 bp DNA ladder, where the bottom band indicates 100 bp and the band above the bottom band indicates 200 bp.

### Example 6—Evidence of D-Loop Formation in ELISA

### Methods

The well of flat-bottom transparent 96-well plate (Greiner) was coated with 5 μg of anti-digoxigenin polyclonal antibody (Enzo Life Sciences) in 50 μL of 14 μM NaCO₃ and 35 μM NaHCO₃ pH 9.6 for 8 h at 4 °C. The well was washed with 100 μL of PBS (1×) and 0.05% v/v Tween-20 three times. The well was blocked with 1% w/v BSA in 50 μL PBS (1×) and 0.05% v/v Tween-20 for 30 min at 37 °C. The well was washed two times with 100 μL of PBS (1×) and 0.05% v/v Tween-20 and then two times with 100 μL of 70 mM Tris-HCl, 10 mM MgCl₂, and 0.05% v/v Tween-20 pH 7.6.

Before removing the wash buffer, in 0.2 mL PCR tube, 0.1 pmol of homologous DNA ((Hom_1 (100 bp) containing 50% mol biotin-16-dUTP), 0.1 pmol 5′-digoxigenin reverse primer (Rev 1 (29 nt), and 10 pmol RecA were mixed in 10 μL of 0.3 mM ATPγS, 70 mM Tris-HCl, 10 mM MgCl₂, and 5 mM DTT pH 7.6. The mixture was incubated at 37 °C for 10 min.

The last wash buffer was removed from the well, and the RecA reaction was diluted to 50 μL with 70 mM Tris-HCl, 10 mM MgCl₂, and 0.05% v/v Tween-20 pH 7.6. The reaction was incubated in the well for 30 min at 37 °C. The well was washed with 100 μL of 70 mM Tris-HCl, 10 mM MgCl₂, and 0.05% v/v Tween-20 pH 7.6 three times. The well was incubated with 50 ng Enhanced Streptavidin-HRP (Kementec) in 50 μL of 70 mM Tris-HCl, 10 mM MgCl₂, and 0.05% v/v Tween-20 pH 7.6 for 30 min at 37 °C. The well was washed with 100 μL of 70 mM Tris-HCl, 10 mM MgCl₂, and 0.05% v/v Tween-20 pH 7.6 three times. The detection was done by incubating with 50 μL of 0.4 mM TMB, 10% v/v DMSO, 50 mM K₂HPO₄, 25 mM trisodium citrate, and 0.006% w/v H₂O₂ pH 5.0 for 1 min at 20 °C. The color development was quenched with 50 uL 0.2 M H2SO₄. The absorbance was read at 450 nm.

### Results

A high-throughput D-loop ELISA detection method was developed. The usage of ELISA that mimicks the layout of a lateral flow was developed. For this, an antibody was immobilized that binds a 5′-label of a primer, followed by incubation of the D-loop, and detection with streptavidin-HRP fusion, see FIG. 7A for a schematic illustration.

The presence and absence of homologous biotinylated DNA was used for comparison where in the presence of DNA a twofold increase in signal was observed, see FIG. 7B. In FIG. 7B, the left bar shows the absorbance of an experiment where homologous DNA containing 50% mol biotin-16-dUTP was used, while the right bar shows the absorbance of an experiment where homologous DNA was replaced by H₂O.

### Example 7—Evidence of D-Loop Formation in a Lateral Flow Assay

### Methods

In a 0.2 mL PCR tube, mixed 0.8 pmol of homologous DNA ((Hom_1 (100 bp) containing 50% mol biotin-16-dCTP), 12.8 pmol 5′FAM reverse primer (Rev 1 (29 nt), and 128 pmol RecA in 10 μL of 0.3 mM ATPγS, 70 mM Tris-HCl, 10 mM MgCl₂, and 5 mM DTT pH 7.6. The mixture was incubated at 37 °C for 10 min. The mixture was diluted to 150 uL in a 1.5 mL Eppendorf tube with PCRD Extraction buffer (Abington Health). A PCRD FLEX lateral flow strip (Abington Health), coated with antidigoxin, antifluorescein and antistreptavidin, was dipped into the solution, incubated at 25 °C, taken out after 10 min, and visually inspected.

### Results

The possibility of DNA detection on a lateral flow strip using a stable D-loop formation was assessed. The concept of the detection is illustrated in FIG. 8A. The presence and absence of ATPγS was used for comparison as ATPγS is known to stabilize D-loop which was observed as a band on the FAM binding site of the lateral flow strip, see FIG. 8B. In FIG. 8B, “D” indicates the digoxigenin binding site, “F” indicates the FAM binding site, “S” indicates the streptavidin binding site. The top strip shows the result of an experiment using ATPγS, while the bottom strip shows the result of an experiment where no ATPγS was used.

### Example 8—Effect of Probe Design on Lateral Flow Assay

### Methods

In a 0.2 mL PCR tube, mixed 0.2 pmol of homologous DNA ((Hom_1 (100 bp) containing 50% mol biotin-16-dUTP), 0.2 pmol probe (5′digoxigenin-Rev 1 (29 nt), 5′digoxigenin-Probe 1 (25 nt)-3′ inverted_dT, or 5′digoxigenin-Probe_2 (60 nt)-3′ C3 spacer) and 2 pmol RecA in 10 μL of 0.3 mM ATPγS, 70 mM Tris-HCl, 10 mM MgCl₂, and 5 mM DTT pH 7.6. The mixture was incubated at 37 °C for 10 min. The mixture was diluted to 150 uL in a 1.5 mL Eppendorf tube with PCRD Extraction buffer (Abington Health). A PCRD FLEX lateral flow strip (Abington Health), coated with antidigoxin, antifluorescein and antistreptavidin, was dipped into the solution, incubated at 25 °C, taken out after 10 min, and visually inspected.

Alternative conditions for the reaction were no additive during the reaction or one of the following: 6 pmol T4 gp32 single-strand binding protein; 5% w/v trehalose, 20% w/v PEG 6000, 100 mM KCl; 80 mM 1-ethyl-3-methylimidazolium acetate (lolitec); combination of all three.

### Results

The stability of D-loop formation in various probe designs as a function of their design was assessed, wherein design refers to a probe that binds to either a terminal end of the biotinylated homologous DNA (5′digoxigenin-Rev 1 (29 nt), FIGS. 9A and B) or to the middle (5′digoxigenin-Probe_1 (25 nt)-3′ inverted_dT, FIGS. 9C and D, or 5′digoxigenin-Probe_2 (60 nt)-3′ C3 spacer, FIGS. 9E and F). In addition, several conditions were tried which are known to stabilize the D-loop since the D-loop that forms at the terminal end (i.e. plectonemic joint) is more stable than a D-loop that forms in the middle of the DNA duplex (i.e. paranemic joint). Those conditions were the presence of single-strand binding protein, PEG, KCl, trehalose, ionic liquid 1-ethyl-3-methylimidazolium acetate, and their combination. The results show that terminal probe binds regardless of the conditions whereas the binding of the middle-binding probe is a function of its length and the additive. The best result was achieved with 60 nt probe (5′digoxigenin-Probe_2 (60 nt)-3′ C3 spacer) that partially overlaps with the primer sequences Fwd_1 and Rev_1 in the presence of the ionic liquid, see FIG. 9F. This was surprising as the use of ionic liquid to improve D-loop stabilization has not been described before.

FIGS. 9A, C, and E show schematic illustrations of the detection concepts of the different probes. FIGS. 9B, D, and F show the results of the respective concepts of FIGS. 9A, C, and E in a lateral flow assay. “D” indicates the digoxigenin binding site, “F” indicates the FAM binding site, “S” indicates the streptavidin binding site. Strip 1 indicates no additive, strip 2 indicates added single-strand binding protein, strip 3 indicates added trehalose, PEG 6000 and KCl, strip 4 indicates added ionic liquid 1-ethyl-3-methylimidazolium acetate, strip 5 indicates added single-strand binding protein, trehalose, PEG, KCl, and ionic liquid 1-ethyl-3-methylimidazolium acetate.

### Example 9—the Specificity of Primer-Probe and Probe with and without ATP-Gamma-S Vs Hapten-Based Strategy

### Methods

The following 25 μL reaction was incubated at 37 °C for 20 min: 1× core mix (TwistDx), 1× reaction buffer (TwistDx), 1× basic E-mix (TwistDx), 450 nM primers (each), 20,000 copies of DNA (Hom_1 (100 bp) or Non_1 (140 bp)), 14 mM magnesium acetate, 0.08 mM dNTP (each), 50% mol/mol biotin-16-dUTP (of total dUTP). Primers were either (1) non-modified primers (Fwd_1 (32 nt) and Rev_1 (29 nt)), (2) mixture of 5′ biotin forward primer (5′ biotin-Fwd_1 (32 nt)) and 5′-digoxigenin reverse primer (5′digoxigenin-Rev 1 (29 nt)) or (3) non-modified primers targeting a sequence different from (1) and (2) (Fwd_2 (30 nt) and Rev_2 (30 nt)). The reaction was purified with GeneJET PCR Purification Kit (ThermoFisher) and quantified with Qubit High-Sense DNA kit (ThermoFisher).

The DNA amplified by the above reaction was added in 50 ng to the following 10 uL reactions: 1.28 pmol probe (5′digoxigenin-Rev 1 (29 nt) or 5′digoxigenin-Probe_2 (60 nt)-C3 spacer), 128 pmol RecA, 0.3 mM ATPγS, 70 mM Tris-HCl, 10 mM MgCl₂, and 5 mM DTT pH 7.6. For different conditions, ATPγS was replaced with 3 mM ATP or 1× Basic E-mix from TwistDx Liquid Basic Kit which contains 50 mM phosphocreatine, 3 mM ATP, and 100 ng/uL creatine kinase. For yet another condition, ATPγS was replaced with the combination of ATPγS with ATP and 1× Basic E-mix where the molar ratio of ATP to ATPγS was 10:1.

The probe binding was quantitatively assessed using an ELISA-based assay. TC-treated flat-bottom transparent 96-well ELISA plate wells were coated with antidigoxin antibody by incubating the well with 50 μg of the antibody in 14 mM Na₂CO₃ and 35 mM NaHCO₃ pH 9.6 for 30 min at 37 °C. The well was washed three times with 1× wash buffer (70 mM Tris-HCl, 10 mM MgCl₂, 0.05% v/v Tween-20, pH 7.0-8.0). The well was blocked with blocking buffer (70 mM Tris-HCl, 10 mM MgCl₂, 1% w/v BSA, pH 7.0-8.0) at 37 °C for 30 minutes and washed four times. Between 1 fmol and 1 pmol of RPA amplicons preincubated with the probe was incubated at 37 °C for 30 minutes and washed three times. 1 μL of 1 μg/mL Enhanced Streptavidin-HRP from Kementec in washing buffer was incubated for 30 minutes at 37 °C and washed three times. 50 μl of 0.04 mg/mL TMB freshly prepared in 50 mM K₂HPO₄, 20 mM sodium citrate, 1% v/v DMSO, and 0.06% w/w H₂O₂ was added to the well and incubated at 25 °C for 2-10 min. 50 μL of 0.2 M H₂SO₄ was added to stop the color development and the absorbance at 450 nm was measured with TECAN multiwell plate reader.

### Results

The extent of sequence-specific binding for the 29 nt primer-probe (5′digoxigenin-Rev 1 (29 nt)) and the 60 nt probe (5′digoxigenin-Probe_2 (60 nt)-C3 spacer) that binds in the middle of the amplicon on RPA-amplified and purified templates with 50% biotin-16-dUTP was assessed. A comparison was made with RPA amplification with hapten-labelled primers (5′ biotin-Fwd_1 (32 nt)) and 5′digoxigenin-Rev_1 (29 nt)). The influence of the replacement ATP-gamma-S with a finite amount of ATP and the ATP with ATP regeneration system (phosphocreatine and creatine kinase), and their combination with ATP-gamma-S was drawn.

FIG. 10A shows the specificity of primer-probe-based (plectonemic joint) D-loop detection using ELISA. It shows the result of an experiment, wherein either DNA template Hom_1 (100 bp) was amplified using Fwd_1 (32 nt) and Rev_1 (29 nt), resulting in the reference “Ho” or wherein DNA template Non_1 (140 bp) was amplified using Fwd_2 (30 nt) and Rev_2 (30 nt), resulting in the reference “No”. The amplified DNA was subjected to a primer-probe (5′digoxigenin-Rev_1 (29 nt), indicated by “Pri”) and recombinase incubation using different ATP/ATPγS inputs. The different ATP/ATPγS inputs are: ATP regeneration via ATP with creatine kinase and phosphocreatine (1×Basic E-mix), indicated by “rA”; ATPγS only, indicated by “Aγ”; ATP only, indicated by “A”; a combination of rA and Aγ, indicated by “rA/Aγ”; a combination of A and Aγ, indicated by “A/Aγ”. The resulting D-loop was subsequently detected using ELISA.

As the primer-probe is non-complementary to the non-homologous DNA template No, no D-loop should be formed, thereby resulting in a signal that can be considered a negative signal. On the contrary, the primer-probe is complementary to the DNA template Ho, resulting in a D-loop and a positive signal. It can be observed in FIG. 10A that for every ATP/ATPγS input condition the positive signal is higher than the negative signal, confirming the presence of a D-loop. A larger difference between a negative signal and a positive signal of a respective ATP/ATPγS input condition can be considered a greater specificity of the probe for the respective condition. Hence, the greatest specificity in FIG. 10A can be observed for the (Pri-)rA/Aγ condition, while the lowest specificity can be observed for the (Pri-)A condition. In general, a higher specificity can be observed when ATPγS is used.

FIG. 10B shows the specificity of probe-based (paranemic joint) D-loop detection using ELISA. It shows the result of an experiment, wherein either DNA template Hom_1 (100 bp) was amplified using Fwd_1 (32 nt) and Rev_1 (29 nt), resulting in the reference “Ho” or wherein DNA template Non_1 (140 bp) was amplified using Fwd_2 (30 nt) and Rev_2 (30 nt), resulting in the reference “No”. The amplified DNA was subjected to a probe (5′digoxigenin-Probe_2 (60 nt)-C3 spacer), indicated by “Pro”) and recombinase incubation using different ATP/ATPγS inputs. The different ATP/ATPγS inputs are: ATP regeneration via ATP with creatine kinase and phosphocreatine (1×Basic E-mix), indicated by “rA”; ATPγS only, indicated by “Aγ”; ATP only, indicated by “A”; a combination of rA and Aγ, indicated by “rA/Aγ”; a combination of A and Aγ, indicated by “A/Aγ”. The resulting D-loop was subsequently detected using ELISA.

As the probe is non-complementary to the non-homologous DNA template No, no D-loop should be formed, thereby resulting in a signal that can be considered a negative signal. On the contrary, the probe is complementary to the DNA template Ho, resulting in a D-loop and a positive signal. It can be observed in FIG. 10B that for every ATP/ATPγS input condition the positive signal is higher than the negative signal, confirming the presence of a D-loop. A larger difference between a negative signal and a positive signal of a respective ATP/ATPγS input condition can be considered a greater specificity of the probe for the respective condition. Hence, the greatest specificity in FIG. 10B can be observed for the (Pro-)A/Aγ condition, while the lowest specificity can be observed for the (Pro-)rA condition. In general, a higher specificity can be observed when ATPγS is used.

Furthermore, comparing the results of the experiments of FIG. 10A with FIG. 10B it can be observed that probe-based D-loop detection has a greater specificity than primer-probe-based D-loop detection. This is surprising since the D-loop that forms at the terminal end (i.e. plectonemic joint) is more stable than a D-loop that forms in the middle of the DNA duplex (i.e. paranemic joint).

FIG. 10C shows a comparison of specificity of D-loop detection with conventional dual-hapten labelled RPA amplicon using ELISA. The conditions Pro-No-A/Aγ, Pro-Ho-A/Aγ, Pri-No-A/Aγ, and Pri-Ho-A/Aγ are taken from the experiments of FIG. 10B and FIG. 10A. In addition, two conventional dual-hapten labelled RPA amplicons were generated as follows: DNA templates Hom_1 (100 bp) and DNA template Non_1 (140 bp) were each separately amplified using 5′ biotin-Fwd_1 (32 nt) and 5′digoxigenin-Rev_1 (29 nt), resulting in the references “5′ DIG-5′ bio-Ho” (a specific amplification product of RPA) and “5′ DIG-5′ bio-No” (i.e. a nonspecific amplification product of RPA) respectively. The resulting product was subsequently detected using ELISA under the same conditions as the experiments described in FIGS. 10A and B.

It can be observed that D-loop detection according to the present invention, in particular when a (stable) paranemic joint is formed, results in an improved signal compared to detection of RPA amplified DNA by conventional dual-hapten labels, which results in a lower relative signal (i.e. the signal of 5′ DIG-5′ bio-Ho subtracted by the signal of 5′ DIG-5′ bio-No).

### Example 10—an RPA Amplicon can be Added Directly into RecA-Probe Binding Immobilized D Loop Detection with High Reproducibility and Specificity

RPA reactions, RecA reactions, and ELISA were performed as described in Example 6 (evidence of D-loop formation in ELISA). Briefly, Fwd_1 and Rev_1 were used to amplify Hom_1 with 30% biotin-16-dUTP in an RPA reaction followed by the serial dilution of the crude RPA reaction in deionized water. The serial dilutions were added in 1 μL volumes to RecA reactions in triplicate with 0.3 mM ATPγS and Probe_2, and subsequently added to ELISA in triplicate (Pro-Ho-Aγ, crude, [dilution factor]). As a negative control, the input amplicon in RecA reactions was 1 ng of amplified and purified Non_1 with 30% biotin-16-dUTP run in triplicate (Pro-No-Aγ). As a positive control for ELISA, the input amplicon in ELISA was 1 ng of amplified and purified 5′ DIG-5′ bio-Ho run in triplicate (5′ DIG-5′ bio-Ho). As a positive control for RecA reaction, the input amplicon in RecA was 1 ng of amplified and purified Hom_1 with 30% biotin-16-dUTP run in triplicate (Pro-Ho-Aγ). As a test for specificity, Non_1 was amplified in RPA with 30% biotin-16-dUTP followed by the serial dilution of the crude RPA reaction in deionized water. The serial dilutions were added in 1 μL volumes to RecA reactions in triplicate with 0.3 mM ATPγS and Probe_2, and subsequently added to ELISA in triplicate (Pro-No-Aγ, crude, [dilution factor]). The results are shown in FIG. 11.

The comparison between the serial dilutions of Hom_1 and Non_1 amplicons in crude RPA reactions shows that the specificity and reproducibility of the D-loop based detection method is retained even after the direct addition of crude RPA product into RecA reaction. The comparison of different dilutions within Hom_1 also shows that the signal is proportional to the amount of added product, indicating once again that the detected species is the RPA amplicon, and the specificity of detection is retained when the same dilutions are compared between Hom_1 and Non_1 amplicons. This example demonstrates that the method according to the invention can be performed as a one-pot reaction.

### Example 11—DrRecA Shows Strand Exchange

A real-time fluorescent assay was developed for recombinase's strand exchange activity. Briefly, 10 pmol of dsDNA annealed from 5′FAM-Fwd_1 and AntiFwd_1-3′BHQ1 was added into the RecA reaction with 10 pmol of Fwd_1. Upon addition of recombinase enzyme and ATP source (E-mix, ATP regeneration system), Fwd_1 replaces its FAM-labelled analogue proximal to BHQ1 in the dsDNA causing an increase in fluorescence, as strand exchange upon D-loop formation occurs in the presence of recombinase and the ATP source. The product can also be run on agarose gel wherein the presence of strand exchange product can be seen as a band of brighter intensity due to the fluorescence of released FAM. Interestingly, strand exchange activity was detected for RecA enzyme from Deinococcus radiodurans (DrRecA) and was not dependent on the presence of the ATP source. This is evident when comparing the fluorescent traces of conditions with and without the ATP source as well as the respective conditions in the agarose gel. As a negative control for the fluorescent assay and agarose gel, a condition without recombinase enzymes was used (indicated by ‘−’ in FIG. 12B). As a positive control for agarose gel, a condition with Escherichia coli RecA with the ATP source was used (indicated by ‘+’ in FIG. 12B). The results of the real-time fluorescent assay are shown in FIG. 12A. The results of the agarose gel experiments are shown in FIG. 12B.

### Example 12—Bridge Flocculation Allows Naked-Eye Detection of Biotinylated DNA

Streptavidin-coated magnetic beads (Cell Signaling Technology) were pulled down with a magnet rack and washed once with the wash buffer (70 mM Tris-HCl, 10 mM MgCl, 0.05% v/v Tween-20, pH 8.0). Purified RPA amplicon of Hom_1 with 30% biotin-dUTP in 10 μL was mixed in 1:1 volume with streptavidin-coated magnetic beads (Cell signaling technology, product no. 5947S) and incubated for 5 min at 37 °C. After washing the beads with the wash buffer twice, 10 μL of enhanced streptavidin-horseradish peroxidase (Kementec) of 1 μg/mL in the wash buffer was added and incubated for 5 min at 37 °C. After washing the beads twice in the wash buffer, a solution of 0.1 mg/mL TMB and 0.6% H₂O₂ in phosphate-citrate buffer of pH 5.0 was added in 300 μL volume and incubated at room temperature for 1 min. As a negative control, a purified product of RPA reaction without template Hom_1 (i.e. no template control) was used.

The distinct difference in coloration between the samples treated with the purified RPA product with and without template DNA indicates that this method can be used to detect biotinylated RPA amplicons with a naked eye after RPA. The results are shown in FIG. 13 (left: no template control; right: template positive).

### Example 13—Stable D-Loop Formation by EcRecA and DrRecA

RPA reactions, RecA reactions, and ELISA were performed as described in Example 9. Specifically, DNA template Hom_1 (100 bp) was amplified via RPA using Fwd_1 and Rev_1. The amplified DNA (“Ho”) was subjected to a probe (5′digoxigenin-Probe_2-C3 spacer) (“Pro-Ho”) and recombinase incubation using different ATP inputs. Equimolar amounts of Pro-Ho probe and Ho template (300 fmol each) were used. The different ATP inputs are: 10 mM Tris-HCl pH 8.0 (no ATP) (0), 1 mM ATP (A), 10 mM dATP (dA), 1 mM ATPγS (Aγ), and ATP regeneration system (rA) (2.5 mM ATP, 300 ng/μL creatine kinase, 50 mM phosphocreatine). The final concentrations of Escherichia coli RecA (EcRecA) and Deinococcus radiodurans RecA (DrRecA) were 0.2 μg/μL and 0.6 μg/μL respectively. The resulting stable D-loop was subsequently detected using ELISA. Results are shown in FIG. 14A (EcRecA) and FIG. 14B (DrRecA).

### REFERENCES

Piepenburg O., Williams C. H, Stemple D. L., Armes N. A., “DNA detection using recombination proteins”, PLoS Biol., 2006 July; 4(7):e204. doi: 10.1371/journal.pbio.0040204.
Lobato I. M. and O'Sullivan C. K., “Recombinase polymerase amplification: Basics, applications and recent advances”, Trends Analyt Chem., 2018 January; 98: p 19-35. doi: 10.1016/j.trac.2017.10.015
Jia T., Yu Y., Wang Y., “A recombinase polymerase amplification-based lateral flow strip assay for rapid detection of genogroup II noroviruses in the field”, Archives of Virology, 2020; 165:2767-2776. doi: 10.1007/s00705-020-04798-x.
Zou X., Dong C., Ni Y., Yuan S., Gao Q., “Rapid detection of strawberry mottle virus using reverse transcription recombinase polymerase amplification with lateral flow strip”, Journal of Virological Methods, 2022; 307:114566. doi: 10.1016/j.jviromet.2022.114566.
Zhu H., Zhang H., Xu Y., Lassakova S., Korabecna M., Neuzil P., “PCR past, present and future”, BioTechniques, 2020, vol. 69, no. 4. doi: 10.2144/btn-2020-0057.
Glökler J., Lim T. S., Ida J., Frohme M., “Isothermal amplifications—a comprehensive review on current methods”, Critical Reviews in Biochemistry and Molecular Biology, 2021, vol. 56, issue 6, p 543-586. doi: 10.1080/10409238.2021.1937927
Daher R. K., Stewart G., Boisinnot M., Boudreau D. K., Bergeron M. G., “Influence of sequence mismatches on the specificity of recombinase polymerase amplification technology”, Molecular and Cellular Probes, 2015, vol. 29, issue 2, p 116-121. doi: doi.org/10.1016/j.mcp.2014.11.005
Liu X., Yan Q., Huang J., Chen J., Guo Z., Liu Z., Cai L., Li R., Wang Y., Yang G., Lan Q., “Influence of design probe and sequence mismatches on the efficiency of fluorescent RPA”, World Journal of Microbiology and Biotechnology, 2019, 35, Article number: 95. doi: doi.org/10.1007/s 11274-019-2620-2
Özay B. and McCalla S. E., “A review of reaction enhancement strategies for isothermal nucleic acid amplification reactions”, Sensors and Actuators Reports, Volume 3, November 2021, 100033. doi: 10.1016/j.snr.2021.100033
Teng Y., Tateishi-Karimata H., Ohyama T., Sugimoto N., “Effect of Potassium Concentration on Triplex Stability under Molecular Crowding Conditions”, Molecules, 2020, 25(2), 387. doi: 10.3390/molecules25020387
Sajid M., Kawde, A., Daud M., “Designs, formats and applications of lateral flow assay: A literature review”, Journal of Saudi Chemical Society, 2015 November; Vol 19, Issue 6, p 689-705. doi: 10.1016/j.jscs.2014.09.001
Singleton and Sainsbury, Dictionary of Microbiology and Molecular Biology, 2d Ed., John Wiley and Sons, NY (1994).
Hale and Marham, The Harper Collins Dictionary of Biology, Harper Perennial, NY (1991).
Higgins, M.; Ravenhall, M.; Ward, D.; Phelan, J.; Ibrahim, A.; Forrest, M. S.; Clark, T. G.; Campino, S. PrimedRPA: Primer Design for Recombinase Polymerase Amplification Assays. Bioinformatics 2019, 35 (4), 682-684. doi: 10.1093/bioinformatics/bty701.
Chen, G.; Dong, J.; Yuan, Y.; Li, N.; Huang, X.; Cui, X.; Tang, Z. A General Solution for Opening Double-Stranded DNA for Isothermal Amplification. Sci. Rep. 2016, 6 (1), 34582. doi: 10.1038/srep34582.
Riddles P. W., Lehman I. R., “The formation of paranemic and plectonemic joints between DNA molecules by the recA and single-stranded DNA-binding proteins of Escherichia coli”, Journal of Biological Chemistry, 1985, Vol. 260(1), p 165-169. doi: 10.1016/S0021-9258(18)89709-5
Martzy, R.; Bica-Schröder, K.; Pálvölgyi, A. M.; Kolm, C.; Jakwerth, S.; Kirschner, A. K. T.; Sommer, R.; Krska, R.; Mach, R. L.; Farnleitner, A. H.; Reischer, G. H. Simple Lysis of Bacterial Cells for DNA-Based Diagnostics Using Hydrophilic Ionic Liquids. Sci. Rep. 2019, 9, 13994. doi: 10.1038/s41598-019-50246-5.
   Munawar M. A., Martin F., Toljamo A., Kokko H., Oksanen E., “RPA-PCR couple: an approach to expedite plant diagnostics and overcome PCR inhibitors.” BioTechniques 69.4 (2020): 270-280. doi: 10.2144/btn-2020-0065
   D'Agata, R.; Palladino, P.; Spoto, G. Streptavidin-Coated Gold Nanoparticles: Critical Role of Oligonucleotides on Stability and Fractal Aggregation. Beilstein J. Nanotechnol. 2017, 8, 1-11. doi: 10.3762/bjnano. 8.1.

1. A method of determining the presence or absence of a target deoxyribonucleic acid (DNA) sequence in a sample, the method comprising:
a. contacting the sample with:
a first primer and a second primer for amplifying the target DNA sequence;
a recombinase;
a DNA-dependent DNA polymerase;
deoxynucleoside triphosphate (dNTP) monomers comprising a first label; and
an oligonucleotide probe comprising a nucleic acid sequence complementary to the target DNA sequence and a second label;
b. amplifying the target DNA sequence to produce a nucleic acid amplification product; and
c. detecting the nucleic acid amplification product, wherein the nucleic acid amplification product comprises a D-loop, the D-loop comprising triple-stranded DNA, the probe, and the recombinase, wherein detecting the nucleic acid amplification product comprises detecting the probe,
wherein contacting the sample with the recombinase and/or the probe may be performed before, during, or after step b of amplifying the target DNA sequence.

2. The method according to claim 1, wherein detecting the nucleic acid amplification product in step c comprises binding one or more first labels of the dNTP monomers of the nucleic acid amplification product to a first binding agent and binding the second label of the probe bound to the nucleic acid amplification product to a second binding agent, preferably wherein the first label does not bind to the second binding agent and the second label does not bind to the first binding agent, and/or wherein the first binding agent is attached to a solid support.

3. The method according to claim 1 or 2, wherein contacting the sample with dNTP monomers comprising a first label comprises contacting the sample with a mixture comprising of from 30 to 70% deoxycytidine triphosphate (dCTP) comprising a first label, of from 30 to 70% deoxyadenosine triphosphate (dATP) comprising a first label, of from 30 to 70% deoxyguanosine triphosphate (dGTP) comprising a first label, of from 30 to 70% deoxythymidine triphosphate (dTTP) comprising a first label, and/or of from 30 to 70% deoxyuridine triphosphate (dUTP) comprising a first label, each based on the total number of moles dNTP of its base type, preferably wherein the first label comprises biotin.

4. The method according to any of claims 1 to 3, further comprising contacting the sample with: a third primer and a fourth primer for amplifying a second target DNA sequence; and a second oligonucleotide probe comprising a nucleic acid sequence complementary to a second target DNA sequence and a third label.

5. The method according claim 4, wherein detecting the nucleic acid amplification product in step c further comprises binding the third label of the second probe bound to the nucleic acid amplification product to a third binding agent, preferably wherein the first label and the third label do not bind to the second binding agent, the second label and the third label do not bind to the first binding agent, and the first label and the second label do not bind to the third binding agent.

6. A method of determining the presence or absence of a target DNA sequence in a sample, the method comprising:
a. contacting the sample with:
a first primer and a second primer for amplifying the target DNA sequence, wherein the second primer comprises a second label and hybridizes to a first strand of DNA of the target DNA sequence;
a recombinase;
a DNA-dependent DNA polymerase; and
an oligonucleotide probe comprising a nucleic acid sequence complementary to the target DNA sequence and one or more nucleotides comprising a first label;
b. amplifying the target DNA sequence to produce a nucleic acid amplification product; and
c. detecting the nucleic acid amplification product, wherein the nucleic acid amplification product comprises a D-loop, the D-loop comprising triple-stranded DNA, the probe, and the recombinase, wherein detecting the nucleic acid amplification product comprises detecting the probe,
wherein contacting the sample with the recombinase and/or the probe may be performed before, during, or after step b of amplifying the target DNA sequence.

7. The method according to claim 6, wherein the probe comprises at least two nucleotides comprising a first label.

8. The method according to claim 6 or 7, wherein detecting the nucleic acid amplification product in step c comprises binding one or more first labels of the nucleotides of the probe to a first binding agent and binding the second label of the second primer bound to the nucleic acid amplification product to a second binding agent, preferably wherein the first label does not bind to the second binding agent and the second label does not bind to the first binding agent, and/or wherein the second binding agent is attached to a solid support.

9. The method according to any of claims 6 to 8, wherein the probe comprises of from 10 to 90%, preferably of from 20 to 80%, nucleotides comprising a first label based on the total number of nucleotides of the probe.

10. The method according to any of claims 6 to 9, further comprising contacting the sample with: a third primer and a fourth primer for amplifying a second target DNA sequence, wherein the fourth primer comprises a third label and hybridizes a first strand of DNA of the second target DNA sequence and a second oligonucleotide probe comprising a nucleic acid sequence complementary to the second target DNA sequence and one or more nucleotides comprising a first label.

11. The method according to claim 10, wherein detecting the nucleic acid amplification product in step c further comprises binding the third label of the fourth primer bound to the nucleic acid amplification product to a third binding agent, preferably wherein the first label and the third label do not bind to the second binding agent, the second label and the third label do not bind to the first binding agent, and the first label and the second label do not bind to the third binding agent.

12. The method according to any of claims 1 to 11, wherein the probe comprises a polymerase blocking group, preferably wherein the blocking group comprises a hydrocarbon spacer, inverted deoxynucleotide, phosphorylation, primary amine, or dideoxynucleotide.

13. The method according to claim 12, wherein the blocking group is at the 3′ end of the probe, wherein the end is of from 0 to 10 nucleotides of the 3′ end nucleotide of the probe including the 3′ end nucleotide.

14. The method according to any of claims 1 to 13, wherein the first label comprises or is biotin or its derivatives and the second label is not or does not comprise biotin or its derivatives, preferably wherein the dNTP monomers comprising the first label comprise biotin-labeled dCTP, biotin-labeled dUTP, biotin-labeled dGTP, biotin-labeled dTTP, and/or biotin-labeled dATP, or preferably wherein the nucleotides comprising the first label comprise a biotin-labeled cytosine nucleotide, biotin-labeled adenine nucleotide, biotin-labeled guanine nucleotide, biotin-labeled thymidine nucleotide, and/or biotin-labeled uracil nucleotide.

15. The method according to any of claims 1 to 14, wherein the first and/or second label is a hapten or a covalently attached label.

16. The method according to any of claims 1 to 15, wherein the second label is a hapten, preferably fluorescein, its derivatives, for example 6-FAM and FITC, digoxigenin, Texas Red, dansyl, Cascade Blue, or dinitrophenol.

17. The method according to any of claims 1 to 16, wherein the second label is at the 5′ end of the probe, at the 3′ end of the probe, or in the middle of the probe, wherein the end is of from 0 to 10 nucleotides of respectively the 5′ or 3′ end nucleotide of the probe, including the end nucleotide, and the middle is of from 0 to 10 nucleotides of the center nucleotide of the probe, including the center nucleotide.

18. The method according to any of claims 1 to 17, wherein the target DNA sequence is amplified by performing an isothermal amplification reaction.

19. The method according to claim 18, wherein the isothermal amplification reaction is a recombinase polymerase amplification (RPA) reaction.

20. The method according to any of claims 1 to 19, wherein the sample is contacted with the probe during step b or after step b, preferably wherein the sample is not contacted with the probe prior to step b.

21. The method according to claim 20, wherein the sample is contacted with the probe after step b and the sample is contacted with an additional amount of recombinase; or wherein the sample is only contacted with the recombinase after step b.

22. The method according to any of claims 1 to 21, wherein the recombinase is a recombinase selected from the group consisting of a RecA, DrRecA, Rad51, RadA, or UvsX recombinase, or a homologue thereof.

23. The method according to any of claims 1 to 22, further comprising contacting the sample with a means for modulating binding of the recombinase or the labeled probe to the nucleic acid amplification product.

24. The method according to any of claims 1 to 23, further comprising adding an ATP analogue, preferably a non-hydrolyzable or hydrolysis-resistant ATP analogue, before, during, or after amplifying the target DNA sequence.

25. The method according to any of claims 1 to 24, further comprises contacting the sample with a double-stranded DNA destabilizing agent and/or a triple-stranded DNA stabilizing agent, preferably wherein the sample comprises the nucleic acid amplification product or an isolated nucleic acid amplification product.

26. The method according to any of claims 1 to 25, further comprising contacting the sample with an RNA-dependent DNA polymerase prior to step a to produce the target DNA sequence.

27. The method according to any one of claims 1 to 26, wherein the dNTP monomers comprising a first label or the nucleotides comprising a first label are respectively biotin-labeled dNTP monomers or biotin-labeled nucleotides, preferably wherein the biotin is covalently linked to respectively the dNTP monomer or the nucleotide via a linker of from 11 to 18 atoms, more preferably 11, 14, 16, or 18 atoms, most preferably 16 atoms.

28. The method according to claim 27, wherein the biotin-labeled dNTP monomers comprise biotin-labeled dCTP and/or biotin-labeled dUTP, preferably biotin-16-dCTP and/or biotin-16-dUTP, or wherein the biotin-labeled nucleotides comprise biotin-labeled cytosine and/or biotin-labeled uracil, preferably biotin-16-cytosine and/or biotin-16-uracil.

29. The method according to any one of claims 1 to 28, wherein the probe comprises of from 30 to 70% dCTP and/or dGTP nucleotides based on the total number of nucleotides of the probe, optionally wherein the nucleotides comprise a first label.

30. The method according to any one of claims 1 to 29, wherein the nucleic acid sequence complementary to the target DNA sequence of the probe or the nucleic acid sequence of the target DNA sequence to which the probe hybridizes does not overlap with the nucleic acid sequence to which the first primer or the second primer hybridizes, or wherein the nucleic acid sequence complementary to the target DNA sequence of the probe or the nucleic acid sequence of the target DNA sequence to which the probe hybridizes partially overlaps with the nucleic acid sequence to which the first primer or the second primer hybridizes, preferably wherein the partial overlap is of from 1 to 10 nucleotides.

31. The method according to any one of claims 1 to 30, wherein the probe comprises a partial nucleic acid sequence or the full nucleic acid sequence of one of the primers used for amplification, preferably wherein the partial sequence comprises at least 1% of the full nucleic acid sequence of one of the primers used for amplification or the first primer used for amplification.

32. The method according to any one of claims 1 to 31, wherein contacting the sample comprises contacting the sample with a smaller amount of probe compared to the amount of the first primer and/or the second primer based on the number of moles, preferably wherein the molar ratio of each of the first primer and the second primer to the probe is of from 1:1 to 100:1.

33. The method according to any one of claims 1 to 32, wherein the first binding agent comprises avidin, streptavidin, neutravidin, an anti-biotin antibody, or a binding agent specific for biotin and/or wherein the second binding agent comprises an antibody.

34. The method according to any one of claims 1 to 33, wherein the first binding agent comprises a nanoparticle, colored (nano)particle, gold (nano)particle, latex, fluorescent compound, luminescent compound, or a magnetic compound.

35. The method according to any one of claims 1 to 34, wherein detecting the nucleic acid amplification product is performed during amplification of the target DNA sequence, preferably wherein the probe, the first label, or the second label comprises a fluorescent compound.

36. The method according to any one of claims 1 to 35, wherein detecting the nucleic acid amplification product comprises using a lateral flow test, lateral flow device, enzyme-linked immunosorbent assay, magnetic immunoassay, or flocculation assay.

37. The method according to claim 36, comprising:
a. providing a lateral flow device comprising a first binding agent capable of binding the first label and a second binding agent capable of binding the second label, preferably wherein the second binding agent is immobilized, preferably immobilized on the lateral flow device;
b. diluting the nucleic acid amplification product, preferably using a buffer with a pH of from 6 to 8, preferably of about 7;
c. applying the diluted nucleic acid amplification product to a lateral flow test strip of the lateral flow device; and
d. detecting the product, if present, on a test zone of the lateral flow strip.

38. The method according to claim 37, wherein the buffer comprises a non-ionic detergent, preferably a non-denaturing non-ionic detergent.

39. The method according to claim 36, comprising combining the nucleic acid amplification product and the first binding agent, wherein the product and the first binding agent are capable of forming a complex which can be detected visually, wherein the complex is formed by flocculation, preferably by flocculation of the first binding agent, more preferably of flocculation of the first binding agent by the nucleic acid amplification product.

40. The method according to claim 39, wherein the presence or relative amount of nucleic acid amplification product is determined by the presence of, or increased flocculation, compared to the flocculation observed in the absence of, or at a lower amount or concentration of, the nucleic acid amplification product.

41. The method according to claim 39 or 40, wherein the first binding agent comprises avidin, streptavidin, neutravidin, an anti-biotin antibody, or a binding agent specific for biotin bound to one or more colored, silver, or gold nanoparticles, preferably wherein the nanoparticles have a diameter of from 5 to 50 nm and/or a silica shell with a diameter of from 2 to 4 nm.

42. The method according to any one of claims 39 to 41, further comprising diluting the complex using a buffer with a pH of from 6 to 8, preferably of about 7, preferably wherein the buffer comprises a non-ionic detergent, preferably a non-denaturing non-ionic detergent.

43. An ex vivo method of diagnosing a disease or disorder, preferably an infectious disease, in a subject, comprising:
a. determining the presence or absence of a target DNA sequence associated with the disease or disorder according to any of claims 1 to 42 in a sample which has been obtained from the subject, wherein the presence indicates a diagnosis of the disease or disorder in the subject.

44. A method of detecting a nucleic acid amplification product comprising a target DNA sequence, the method comprising:
a. contacting the nucleic acid amplification product with a recombinase and an oligonucleotide probe comprising a nucleic acid sequence complementary to the target DNA sequence and a label, thereby forming in the nucleic acid amplification product a D-loop comprising triple-stranded DNA, the probe, and the recombinase; and
b. detecting the nucleic acid amplification product, comprising detecting the probe.

45. The method according to claim 44, wherein detecting the nucleic acid amplification product comprises binding the label of the probe bound to the nucleic acid amplification product to a binding agent, preferably wherein the binding agent is attached to a solid support, more preferably wherein detecting the nucleic acid amplification product comprises using a lateral flow test, lateral flow device, enzyme-linked immunosorbent assay, magnetic immunoassay, or flocculation assay.

46. The method according to any one of claims 1 to 45, wherein the method is nuclease-free, preferably nuclease-free and protease-free.

47. A nuclease-free composition comprising:
dNTP monomers comprising a first label and an oligonucleotide probe comprising a second label; or
a first primer and a second primer for amplifying a target DNA sequence, wherein the second primer comprises a second label and is capable of hybridizing to a first strand of DNA of the target DNA sequence; and an oligonucleotide probe comprising a nucleic acid sequence complementary to the target DNA sequence and one or more nucleotides comprising a first label, preferably wherein the probe is capable of binding or hybridizing to the first strand or the second strand of DNA of the target DNA sequence, the second strand being complementary to the first strand.

48. A lateral flow device, comprising a lateral flow strip, comprising:
a sample application zone;
a reagent zone downstream of the sample application zone and in fluid communication with the sample application zone, wherein the reagent zone comprises a non-immobilized first binding agent that binds to a first label;
a test zone downstream of the reagent zone and in fluid communication with the reagent zone, wherein the test zone comprises an immobilized second binding agent that binds to a second label; and
a control zone downstream of the test zone and in fluid communication with the test zone.

49. The device according to claim 48, wherein the reagent zone comprises a recombinase and/or a dried RPA composition for amplifying a target DNA sequence, preferably comprising the composition according to claim 47.

50. The device according to claim 48 or 49 for use in the method according to any one of claims 1 to 46.

51. A nuclease-free kit of parts comprising:
a. optionally, a first primer and a second primer for amplifying a target DNA sequence;
b. a recombinase;
c. a DNA-dependent DNA polymerase;
d. dNTP monomers comprising a first label; and
e. an oligonucleotide probe comprising a nucleic acid sequence complementary to the target DNA sequence and a second label; and optionally
f. a lateral flow device or the device according to claim 48 or 49; and optionally
g. instructions for carrying out the method according to any one of claims 1 to 46.

52. A nuclease-free kit of parts comprising:
a. a first primer and a second primer for amplifying a target DNA sequence, wherein the second primer comprises a second label and hybridizes to a first strand of DNA of the target DNA sequence;
b. a recombinase;
c. a DNA-dependent DNA polymerase; and
d. an oligonucleotide probe comprising a nucleic acid sequence complementary to the target DNA sequence and one or more nucleotides comprising a first label, preferably wherein the probe hybridizes to the first strand or the second strand of DNA of the target DNA sequence, the second strand being complementary to the first strand; and
optionally
e. a lateral flow device or the device according to claim 48 or 49; and optionally
f. instructions for carrying out the method according to any one of claims 1 to 46.

53. The composition according to claim 47 or the kit of parts according to claim 51 or 52, further being protease-free and/or for use in the method according to any one of claims 1 to 46.

54. Use of the device according to claim 48 or 49 or the kit of parts according to claim 51 or 52 for determining the presence or absence of a pathogen, preferably a human, animal, or plant pathogen, or a target DNA sequence associated with a disease or disorder or the onset thereof.

55. The method according to claim 43 or the use according to claim 54, wherein the disease or disorder is cancer, autoimmune disease or disorder, hereditary disease or disorder, pain, respiratory disease or disorder, cardiovascular disease or disorder, infectious disease or disorder, thyroid disease or disorder, skin disease or disorder, diabetes, obesity, kidney disease or disorder, gastrointestinal disease or disorder, neurodegenerative disease or disorder, musculoskeletal disease or disorder, metabolic disease or disorder, or mental illness, preferably cancer, autoimmune disease or disorder, or infectious disease or disorder.

56. The use according to claim 54, wherein the target DNA sequence associated with a disease or disorder or the onset thereof comprises a DNA sequence expressing tumour antigens, tumour markers, or tumour suppressors.

57. Use of a device according to claim 48 or 49 or a kit of parts according to claim 51 or 52 for determining the presence or absence of a target DNA sequence in a diagnostic sample, a veterinary sample, a pharmaceutical sample, a cosmetics sample, a food sample, a beverage sample, a feed sample, an agricultural sample, a soil sample, an air sample, a water sample, a waste sample, an effluent sample, or a sewage sample.
